(19) United States
(12) Patent Application Publication
(10) Pub. No.: US 2023/0109130 A1
(43) Pub. Date: Apr. 6, 2023

(54) METHODS AND SYSTEMS RELATED TO HIGHLY SENSITIVE ASSAYS AND DELIVERING CAPTURE OBJECTS

(71) Applicant: Quanterix Corporation, Billerica, MA (US)

(72) Inventors: David C. Duffy, Arlington, MA (US); Nicholas Collier, Burwell (GB); Cheuk W. Kan, Waltham, MA (US); Carmen I. Tobos, Seattle, WA (US); David M. Rissin, Lexington, MA (US); Alexander D. Wiener, Tewksbury, MA (US); Ray Meyer, Medford, MA (US); Christopher Warwick, Royston (GB); Roger Millington, Huntingdon (GB); Danielle M. Svancara, Cambridge, MA (US)

(73) Assignee: Quanterix Corporation, Billerica, MA (US)

(21) Appl. No.: 17/965,199

(22) Filed: Oct. 13, 2022

Related U.S. Application Data
(63) Continuation of application No. PCT/US2021/027347, filed on Apr. 14, 2021.
(60) Provisional application No. 63/010,625, filed on Apr. 15, 2020.
(60) Provisional application No. 63/010,613, filed on Apr. 15, 2020.

(51) Int. Cl. G01N 33/543; G01N 15/06; G01N 35/08; G01N 35/00; G01N 33/68

(52) U.S. Cl. CPC G01N 33/54386; G01N 15/0612; G01N 35/08; G01N 35/0098; G01N 33/6869; G01N 2035/0441

(57) ABSTRACT

Methods and systems for capture object-based assays, including for determining a measure of the concentration of an analyte molecule or particle in a fluid sample, are described. The methods and systems may relate to high sensitivity detection of analytes, sometimes using assay conditions and sample handling that result in the capture and detection of a high percentage of the analyte molecules or particles in a fluid sample using relatively few capture objects. Apparatuses and methods for immobilizing capture objects with respect to assay sites, in some instances with unexpectedly high efficiencies are also described. Some such apparatuses involve the use of force fields and fluid meniscus forces, alone or in combination, to facilitate or improve capture object immobilization. Also described are techniques for utilizing a relatively high percentage of capture objects in an assay sample, such as by using disclosed sample washing techniques, imaging systems, and analysis procedures that can reduce capture object loss.

### RELATED APPLICATIONS

This application is a Continuation of International Patent Application Serial No. PCT/US2021/027347, filed Apr. 14, 2021, which claims priority under 35 U.S.C. § 119(e) to U.S. Provisional Patent Application Ser. No. 63/010,613, filed Apr. 15, 2020, and U.S. Provisional Patent Application Ser. No. 63/010,625, filed Apr. 15, 2020, each of which is incorporated herein by reference in its entirety for all purposes.

### TECHNICAL FIELD

Methods and systems for analyte capture assays, including for determining a measure of the concentration of an analyte molecule or particle in a fluid sample, are generally described.

### BACKGROUND

The ability to precisely measure target analyte molecules (e.g., proteins and nucleic acids) is important in many fields, including clinical diagnostics, testing of blood banks, research, and the analysis of biochemical pathways. Assays and related systems/apparatuses exist for the detection of single molecules of target analyte molecules, which may utilize beads or other capture objects. One category of such assays with generally high sensitivity are digital enzyme-linked immunosorbent assays (“digital ELISA”). Certain digital ELISA assays involve capturing proteins or other target analytes on microscopic beads (or other capture objects), labeling the target analytes with an enzyme, isolating the beads in arrays of small wells, and detecting bead-associated enzymatic activity using fluorescence imaging. Spatial localization and/or separation of individual beads, for example in arrays, can allow for the determination of the single molecule signal associated the beads, enabling a measure of the number and/or concentration of the target analyte to be determined at very low values. Various other analyte capture-based assays, and related systems and apparatuses, have also been developed to determine a measure of the number and/or concentration of analyte molecules in a fluid sample, wherein the analyte molecules are captured on beads or other capture objects. However, there is a continued need for methods, techniques, and systems that improve the sensitivity of such assays.

### SUMMARY

Methods and systems for capture object-based assays, including for determining a measure of the concentration of an analyte molecule or particle in a fluid sample, are described. The methods and systems may relate to high sensitivity detection of analytes, sometimes using assay conditions and sample handling that result in the capture and detection of a high percentage of the analyte molecules or particles in a fluid sample using relatively few capture objects. Apparatuses and methods for immobilizing capture objects with respect to assay sites, in some instances with unexpectedly high efficiencies are also described. Some such apparatuses involve the use of force fields and fluid meniscus forces, alone or in combination, to facilitate or improve capture object immobilization. Also described are techniques for utilizing a relatively high percentage of capture objects in an assay sample, such as by using disclosed sample washing techniques, imaging systems, and analysis procedures that can reduce capture object loss.

The subject matter of the present invention involves, in some cases, interrelated products, alternative solutions to a particular problem, and/or a plurality of different uses of one or more systems and/or articles.

In some embodiments, methods for immobilizing capture objects with respect to assay sites are described. In some embodiments, the method comprises delivering capture objects in proximity to assay sites on a surface; generating a force field in proximity to the surface tending to act on the capture objects such that the capture objects move toward the surface; flowing a fluid plug containing the capture objects in a first direction such that a first direction receding meniscus of the fluid plug flows across at least some of the assay sites; flowing the fluid plug in a second, different direction such that a second direction receding meniscus of the fluid plug flows across at least some of the assay sites; and immobilizing at least some of the capture objects subjected to the steps of flowing the fluid plug in the first direction and/or flowing the fluid plug in the second direction with respect to the assay sites.

In some embodiments, the method comprises delivering capture objects in proximity to assay sites on a surface; generating a force field in proximity to the surface tending to act on the capture objects such that the capture objects move toward the surface; flowing a fluid plug containing the capture objects across at least some of the assay sites one or more times; and immobilizing at least some of the capture objects subjected to the flowing step with respect to the assay sites; wherein at least 20% of the total number of capture objects delivered in proximity to the assay sites are immobilized during the flowing step.

In some embodiments, apparatuses for immobilizing capture objects with respect to assay sites on a surface of an assay consumable are described. In some embodiments, the apparatus comprises a capture object applicator configured to apply capture objects to the surface of the assay consumable or in proximity to the surface; a force field generator adjacent to the assay consumable when present, and configured to generate a force field in proximity to the surface; a fluid injector configured to generate a fluid plug having a first meniscus and a second meniscus each adjacent to an immiscible fluid when on the surface of the assay consumable; a fluid pump capable of moving fluid across at least part of the surface; and a controller comprising one or more processors configured to modulate the fluid pump to move the fluid plug bi-directionally across at least part of the surface.

In some embodiments, apparatuses for associating capture objects with respect to assay sites on a surface of an assay consumable are described. In some embodiments, the apparatus comprises a capture object applicator configured to apply capture objects to the surface of the assay consumable or in proximity to the surface; a force field generator adjacent to the assay consumable when present, and configured to generate a force field in proximity to the surface, wherein the force field is a non-uniform electric field capable of applying a dielectrophoretic force to polarizable dielectric capture objects; a fluid injector configured to generate a fluid plug having a first meniscus and a second meniscus each adjacent to an immiscible fluid when on the surface of the assay consumable; a fluid pump capable of moving fluid across at least part of the surface; and a controller comprising one or more processors configured to modulate the fluid pump to move the fluid plug bi-directionally across at least part of the surface.

In some embodiments, the apparatus for associating capture objects with assay sites on a surface of an assay consumable comprises a capture object applicator configured to apply capture objects to the surface of the assay consumable or in proximity to the surface; a power source; electrically conductive solids in conductive or inductive electrical communication with the power source that are adjacent or opposite a surface of the assay consumable when present; a fluid injector configured to generate a fluid plug; and a controller comprising one or more processors configured to initiate application of a voltage to at least some of the electrically conductive solids by the power source to (a) generate a non-uniform electric field in proximity to the surface capable of applying a dielectrophoretic force to polarizable dielectric capture objects, and (b) generate an electric field that moves a fluid plug across at least part of the surface.

In some embodiments, methods for determining a measure of a concentration of analyte molecules or particles in a fluid sample are described. In some embodiments, the method comprises exposing capture objects, each having affinity for a particular type of analyte molecule or particle, to a solution containing or suspected of containing at least one type of analyte molecule or particle, wherein the number of capture objects exposed to the solution containing or suspected of containing the analyte molecules or particles is less than or equal to 50,000; immobilizing analyte molecules or particles of the particular type of analyte molecule or particle with respect to the capture objects such that at least some of the capture objects associate with at least one of the particular type of analyte molecule or particle from the fluid sample and a statistically significant fraction of the capture objects do not associate with any of the particular type of analyte molecule or particle from the fluid sample; determining a measure indicative of the number or fraction of capture objects associated with at least one of the particular type of analyte molecule or particle from the fluid sample; and determining a measure of the concentration of the particular type of analyte molecule or particle in the fluid sample based at least in part on the measure indicative of the number or fraction of capture objects determined to be associated at least one of the particular type of analyte molecule or particle.

In some embodiments, the method comprises exposing capture objects, each having affinity for a particular type of analyte molecule or particle, to a solution containing or suspected of containing the at least one type of analyte molecules or particle, wherein the number of capture objects exposed to the solution containing or suspected of containing the analyte molecules or particles is less than or equal to 50,000; immobilizing analyte molecules or particles of the particular type of analyte molecule or particle with respect to the capture objects such that at least some of the capture objects associate with at least one of the particular type of analyte molecule or particle from the fluid sample; determining a measure indicative of the number or fraction of capture objects associated with at least one of the particular type of analyte molecule or particle from the fluid sample; and based upon the measure indicative of the number or fraction of capture objects associated with at least one of the particular type of analyte molecule or particle from the fluid sample, either determining a measure of the concentration of the particular type of analyte molecule or particle in the fluid sample based at least in part on the measure indicative of the number or fraction of capture objects determined to be associated at least one of the particular type of analyte molecule or particle, or determining a measure of the concentration of the particular type of analyte molecule or particle in the fluid sample based at least in part on a measured intensity level of a signal that is indicative of the presence of a plurality of the particular type of analyte molecules or particles.

In some embodiments, the method comprises exposing capture objects, each having affinity for a particular type of analyte molecule or particle, to a solution containing or suspected of containing at least one type of analyte molecule or particle; immobilizing analyte molecules or particles of the particular type of analyte molecule or particle with respect to the capture objects such that at least some of the capture objects associate with at least one of the particular type of analyte molecule or particle from the fluid sample and a statistically significant fraction of the capture objects do not associate with any of the particular type of analyte molecule or particle from the fluid sample; spatially segregating at least 25% of the capture objects subjected to the immobilizing step into a plurality of separate locations; addressing at least a portion of the plurality of locations subjected to the spatially segregating step to determine a measure indicative of the number or fraction of capture objects associated with at least one of the particular type of analyte molecule or particle from the fluid sample; and determining a measure of the concentration of the particular type of analyte molecule or particle in the fluid sample based at least in part on the measure indicative of the number or fraction of capture objects determined to be associated at least one of the analyte molecule or particle.

In some embodiments, the method comprises exposing capture objects, each having affinity for a particular type of analyte molecule or particle, to a solution containing or suspected of containing at least one type of analyte molecule or particle, wherein the number of capture objects exposed to the solution containing or suspected of containing the analyte molecules or particles is less than or equal to 50,000; immobilizing analyte molecules or particles of the particular type of analyte molecule or particle with respect to the capture objects such that at least some of the capture objects associate with at least one of the particular type of analyte molecule or particle from the fluid sample, while a statistically significant fraction of the capture objects do not associate with any of the particular type of analyte molecule or particle from the fluid sample; immobilizing at least one binding ligand with respect to at least some of the particular type of analyte molecules or particles associated with a capture object; exposing the at least one immobilized binding ligand to a precursor labeling agent such that the precursor labeling agent is converted to a labeling agent that becomes immobilized with respect to the capture object to which the binding ligand is immobilized; determining a measure indicative of the number or fraction of capture objects comprising at least one immobilized labeling agent; and determining a measure of the concentration of the particular type of analyte molecule or particle in the fluid sample based at least in part on the measure indicative of the number or fraction of capture objects determined to comprise at least one immobilized labeling agent.

In some embodiments, apparatuses for imaging an array of assay sites on the surface of an assay consumable are described. In some embodiments, the apparatus comprises an imaging system comprising a detector and optics having a fixed field of view greater than an area containing the array of assay sites; a computer-implemented control system configured to receive information from the imaging system and analyze an entirety of the area containing the array of assay sites; wherein the assay sites have a volume of between 10 attoliters and 100 picoliters.

In some embodiments, methods for performing assays for detecting analyte molecules or particles in a fluid sample are described. In some embodiments, the method comprises providing between 1,000 and 200,000 capture objects; preparing the capture objects and analyte molecules or particles from the fluid sample for detection by performing one or more processes comprising each of the following: (1) mixing the capture objects and analyte molecules or particles in a liquid to form a capture object suspension, and (2) applying a force to the capture object suspension to remove the liquid from the capture object suspension, wherein applying the force does not comprise applying a negative pressure to the capture object suspension via fluidic connection of the capture object suspension to a source of vacuum tending to move the liquid; wherein: the preparing step results in prepared capture objects, at least some of which are associated with the analyte molecules or particles from the fluid sample and a statistically significant fraction of which are not associated with any analyte molecule or particle; and the total number of prepared capture objects is greater than or equal to 90% of the capture objects in the providing step; and determining a measure of the concentration of analyte molecules or particles in the fluid sample based at least in part on the measure indicative of the number or fraction of capture objects determined to be associated at least one analyte molecule or particle.

In some embodiments, apparatuses for performing an assay are described. In some embodiments, the apparatus comprises: a sample washer configured to prepare magnetic beads and analyte molecules or particles from the fluid sample for detection; a bead applicator configured to apply the magnetic beads to a surface of an assay consumable or in proximity to the surface, the surface comprising reaction vessels; a magnetic field generator configured to be adjacent to the assay consumable and configured to generate a magnetic field in proximity to the surface; and a fluid injector configured to generate a fluid plug having a first meniscus and a second meniscus each adjacent to an immiscible fluid when on the surface of the assay consumable; a fluid pump capable of moving fluid across the surface of the assay consumable; an imaging system comprising a detector and optics having a fixed field of view greater than an area defined by the array of reaction vessels; and a controller comprising one or more processors configured to modulate the fluid pump to move fluid across the surface of the assay consumable.

In some embodiments, methods for determining a measure of the concentration of analyte molecules or particles in a fluid sample are provided. In some embodiments, the method comprises: exposing magnetic beads to a solution containing or suspected of containing at least one type of analyte molecule or particle; immobilizing analyte molecules or particles with respect to the magnetic beads such that at least some of the magnetic beads associate with at least one analyte molecule or particle from the fluid sample and a statistically significant fraction of the magnetic beads do not associate with any analyte molecule or particle from the fluid sample; removing the solution from at least a portion of the magnetic beads subjected to the immobilizing step; delivering the magnetic beads in proximity to reaction vessels on a surface; generating a magnetic field in proximity to the surface tending to act on the capture objects such that the capture objects move toward the surface; flowing a fluid plug containing the magnetic beads such that a receding meniscus of the fluid plug flows across at least some of the reaction vessels; inserting at least a portion of the magnetic beads into the reaction vessels; imaging an entirety of the reaction vessels following the inserting step; analyzing an entirety of the reaction vessels subjected to the imaging step to determine a measure indicative of the number or fraction of magnetic beads associated with an analyte molecule or particle from the fluid sample; and determining a measure of the concentration of analyte molecules or particles in the fluid sample based at least in part on the measure indicative of the number or fraction of beads determined to be associated at least one analyte molecule or particle.

In some embodiments, the method comprises: exposing capture objects to a solution containing or suspected of containing at least one type of analyte molecule or particle; immobilizing analyte molecules or particles with respect to the capture objects such that at least some of the capture objects associate with at least one analyte molecule or particle from the fluid sample and a statistically significant fraction of the capture objects do not associate with any analyte molecule or particle from the fluid sample; removing the solution from at least a portion of the capture objects subjected to the immobilizing step while retaining at least 80% of the capture objects subjected to the immobilizing step; delivering at least 80% of the capture objects subjected to the removing step in proximity to assay sites on a surface; immobilizing at least 20% of the capture objects subjected to the delivering step with respect to the assay sites; imaging at least 80% of the assay sites; analyzing at least 75% of the assay sites subjected to the imaging step to determine a measure indicative of the number or fraction of magnetic capture objects associated with an analyte molecule or particle from the fluid sample; and determining a measure of the concentration of analyte molecules or particles in the fluid sample based at least in part on the measure indicative of the number or fraction of capture objects determined to be associated at least one analyte molecule or particle.

In some embodiments, the method comprises determining a measure of the concentration of analyte molecules or particles in a fluid sample at a level of detection of less than 2×10⁻¹⁸ M.

In some embodiments, methods for immobilizing a capture object with respect to an assay site is described. In some embodiments, the method comprises delivering a capture object in proximity to an assay site on a surface; applying an external force to the capture object subjected to the delivering step such that a distance between the capture object and the assay site is decreased; flowing a fluid plug containing the capture object such that a receding meniscus of the fluid plug flows across the assay site; and immobilizing the capture object with respect to the assay site via application of force contributed at least in part by the receding meniscus.

In some embodiments, methods for associating a capture object with respect to an assay site are described. In some embodiments, the method comprises delivering a capture object in proximity to an assay site on a surface; applying an external force to the capture object subjected to the delivering step such that a distance between the capture object and the assay site is decreased, wherein the external force is a dielectrophoretic force; flowing a fluid plug containing the capture object such that a receding meniscus of the fluid plug flows across the assay site; and associating the capture object with respect to the assay site via application of force contributed at least in part by the receding meniscus.

In some embodiments, methods for associating a capture object with an assay site are described. In some embodiments, the method comprises delivering a capture object in proximity to an assay site on a surface by flowing a fluid plug containing the capture object to the assay site using digital microfluidics techniques, generating a non-uniform electric field to apply an external dielectrophoretic force to the capture object subjected to the delivering step such that a distance between the capture object and the assay site is decreased, and associating the capture object with respect to the assay site via application of force contributed at least in part by the dielectrophoretic force.

In some embodiments, methods for immobilizing capture objects with respect to assay sites are described. In some embodiments, the method comprises delivering fluid containing capture objects in proximity to assay sites on a surface; generating a force field in proximity to the surface tending to act on the capture objects such that the capture objects move toward the surface; applying a lateral force to the capture objects by adjusting a lateral distribution of the force field; and immobilizing at least some of the capture objects with respect to the assay sites at least in part via the applied lateral force, wherein at least 20% of the total number of capture objects delivered in proximity to the assay sites are immobilized during the applying step. In some embodiments, kits are provided. In some embodiments, the kit comprises capture objects including a binding surface having affinity for the analyte molecule or particle, wherein a level of detection of a first assay using 5,000 capture objects identical to those in the kit has a level of detection that is at least 50% lower than the level of detection of a second assay using 500,000 capture objects identical to those in the kit, wherein: the first assay comprises a step of incubating the capture objects with the analyte molecule or particle for a first period of time, the second assay comprises a step of incubating the capture object with the analyte molecule or particle for a second period of time, the first period of time being 100 times greater than the second period of time, and the first assay and the second assay are performed under otherwise identical conditions.

In some embodiments, the kit comprises a packaged container for an analyte detection assay, comprising between 50,000 and 5,000,000 capture objects each including a binding surface having affinity for the analyte and having an average diameter of between 0.1 micrometers and 100 micrometers, wherein the analyte detection assay can be performed at a level of detection of less than or equal to 50×10⁻¹⁸ M.

In some embodiments, compositions are provided. In some embodiments, the composition comprises an isolated fluid having a volume of between 10 and 1000 microliters; at least one type of analyte molecule or particle present in a concentration of between 0.001 aM and 10 pM; and between 100 and 50,000 capture objects including a binding surface having affinity for the at least one type of analyte molecule or particle.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a block diagram showing the components of an embodiment of an apparatus for performing at least a part of an assay comprising at least an assay consumable handler, a capture object applicator, a fluid injector, a fluid pump, and a controller, according to certain embodiments;

FIG. 2A is a schematic diagram of an exemplary method of immobilizing capture objects with respect to assay sites on a surface, according to certain embodiments;

FIG. 2B is a schematic diagram of an exemplary method of immobilizing capture objects with respect to assay sites on a surface in the presence of a force field, according to certain embodiments;

FIG. 2C is a schematic diagram of an exemplary method of immobilizing capture objects with respect to assay sites on a surface in the presence of a force field during the flow of a fluid plug comprising a receding meniscus at a point in time when the receding meniscus begins to pass over the assay sites, according to certain embodiments;

FIG. 2D shows the schematic diagram of FIG. 2C at a later point in time when the receding meniscus has passed over all of the assay sites, according to certain embodiments;

FIG. 2E is a schematic diagram of an exemplary method of immobilizing capture objects with respect to assay sites on a surface in the presence of a force field during the flow of a fluid plug comprising a receding meniscus, according to certain embodiments;

FIG. 2F is a schematic diagram of the flow of a fluid plug comprising a receding meniscus, according to certain embodiments;

FIG. 2G is a schematic diagram of an exemplary method of immobilizing capture objects with respect to assay sites on a surface in the presence of a force field during the flow of multiple fluid plugs, according to certain embodiments;

FIGS. 3A-3B are schematic diagrams of an apparatus for immobilizing capture objects with respect to assay sites on the surface of an assay consumable operatively coupled to an assay consumable handler, according to certain embodiments;

FIGS. 3C-3D show a top view schematic illustration of capture objects in proximity to a surface comprising assay sites surrounded by a network of electrically conductive solids in the absence (FIG. 3C) and presence (FIG. 3D) of a repulsive dielectric force, according to certain embodiments;

FIGS. 4A-4F are schematic diagrams showing illustrating exemplary assay consumable handlers, according to certain embodiments; FIG. 5 is a schematic diagram of an apparatus for imaging an array of assay sites on the surface of an assay consumable, according to certain embodiments;

FIGS. 6A-6B are schematic flow diagrams depicting one embodiment of a capture-object based assay for detecting analyte molecules or particles, according to certain embodiments;

FIGS. 7A-7B are top view and perspective schematic diagrams of an exemplary microfluidic apparatus for use for detecting analyte molecules or particles, according to certain embodiments;

FIG. 8 is a schematic diagram of a sample washer apparatus, according to certain embodiments;

FIG. 9 is a plot of a modeled increase in the ratio of captured protein molecules to beads for assays using 5,000 beads compared to 500,000 beads, assuming 274,000 capture antibodies per bead, as a function of dissociation constant (K_{D}) of a capture antibody-antigen interaction, according to certain embodiments;

FIG. 10 is a schematic of a magnetic-meniscus sweeping (MMS) method for bead loading in arrays of microwells, according to certain embodiments;

FIG. 11 is a plot of AEB against [IL-17A] at two bead numbers and two incubation times, according to certain embodiments;

FIG. 12 is a plot of AEB against [IL-17A] as a function of the number of capture beads for a 4 h incubation of beads and sample, according to certain embodiments;

FIG. 13 is a plot of AEB against [IL-17A] for bead numbers ranging from 4,530 to 32,000 at 4 hour sample incubation, according to certain embodiments;

FIG. 14A shows AEB as a function of sample incubation time at [IL-17A]=1.2 fM using 15,000 beads, according to certain embodiments;

FIG. 14B is a plot of AEB against [IL-17A] as a function of sample incubation time using 15,000 beads, according to certain embodiments;

FIG. 15 is a plot of AEB against [IL-17A] as a function of sample volume using 15,000 beads and an incubation time of 6 hours; according to certain embodiments;

FIG. 16 is a plot of AEB against [IL-17A] for standard ELISA (500,000 beads; 100 μL sample; 30 min incubation) and digital ELISAs using low bead numbers (5,453, 2,726, or 1,363 beads; 200 μL sample; 24 h incubation), according to certain embodiments;

FIG. 17 is plots of AEB against [IL-17A] using 5,000 beads and: a) 100 μL of sample incubated for 6 h (open squares); and, b) 250 μL of sample incubated for 24 h (closed circles), according to certain embodiments;

FIG. 18 is a plot of spike recovery of IL-17A from a serum sample at two spiked concentrations as a function of number of beads, according to certain embodiments;

FIGS. 19A-19B are scatter plots of [IL-17A] determined using either standard digital ELISA or the low bead digital ELISA in serum and plasma samples, according to certain embodiments;

FIG. 20 is a plot showing the correlation of quantified serum and plasma samples using standard digital ELISA and low bead/high efficiency digital ELISA, according to certain embodiments;

FIG. 21 are plots of AEB against concentration of IL-17A, IL-12p70, p24, IFN-α, IL-4, and PSA using digital ELISAs adjusted for low bead numbers (open circles) and standard digital ELISA (filled squares), according to certain embodiments;

FIG. 22 is a plot of AEB against concentration of IL-12p70 spiked into diluted serum for standard ELISA (400,000 beads; 100 μL sample; 30 min incubation) and digital ELISAs adjusted for low bead numbers (5,368, 2,684, or 1,342 beads; 200 μL sample; 24 h incubation), according to certain embodiments;

FIG. 23 is a plot of AEB against concentration of p24 spiked into diluted serum for standard ELISA (300,000 beads; 125 μL sample; 30 min incubation) and digital ELISAs adjusted for low bead numbers (5,259, 2,625, or 1,313 beads; 125 μL sample; 24 h incubation), according to certain embodiments; and

FIG. 24 is an image of an array of microwells positioned over a magnet, according to certain embodiments.

### DETAILED DESCRIPTION

Methods and systems for analyte capture based assays, including for determining a measure of the concentration of an analyte molecule or particle in a fluid sample, are described.

The methods and systems described may provide high sensitivity detection of analytes (e.g., at femtomolar, attomolar, zeptomolar, or lower levels), in some instances using assay conditions and sample handling techniques that result in the capture and detection of a high percentage of analyte molecules or particles in an assay sample using relatively few capture objects with respect to typical conventional assays. Also described are apparatuses and methods for immobilizing capture objects (e.g., beads) with respect to assay sites (e.g., reaction vessels such as microwells), in some instances with unexpectedly high efficiencies. Some such apparatuses involve the use of force fields (e.g., magnetic fields) and fluid meniscus forces, alone or in combination, to help facilitate or improve capture object immobilization. Also described are techniques for utilizing a relatively high percentage of capture objects in an assay sample, such as by using described washing techniques, imaging systems, and analysis procedures that can reduce capture object loss.

In some embodiments, apparatuses involving assay consumables with surfaces comprising assays sites, capture object applicators, force field generators, fluid handling components (e.g., fluid injectors and pumps), controllers, and optionally, certain assay consumable handlers, imaging systems, and sample washers (e.g., non-vacuum-based sample washers) are described. The apparatuses may be configured to perform highly sensitive assays (e.g., digital ELISAs). In some instances the apparatuses and related methods involve the use of fewer capture objects (e.g., fewer than 50,000, fewer than 10,000, fewer than 5,000, or fewer) compared to typical conventional assays, with resulting advantages in certain cases that are unexpected. Certain methods, and related apparatus components and configurations described can provide non-limiting solutions to challenges associated with the use of such low numbers of capture objects. For example, certain disclosed techniques and associated apparatuses relate to retaining sufficient numbers of capture objects for generating adequate signal and to capturing sufficient numbers of analytes. One exemplary technique relates to facilitating effective immobilization of capture objects (e.g., insertion of beads), which can be important in low capture object number regimes described. Some embodiments relate to system configurations and methods involving generating force fields (e.g., magnetic fields) in proximity to capture objects (e.g., magnetic beads) near assay sites, and flowing a fluid plug containing the capture objects (and the plug's receding meniscus) across the assay sites (e.g., bi-directionally). Other techniques described relate to improvement of assay sensitivity, improved image detection, and analysis and sample handling (e.g., liquid removal techniques, sample incubation).

While conventional highly sensitive assays such as conventional digital ELISAs can have sensitivities to permit the measurement of analytes previously undetectable, even greater sensitivity (e.g., low attomolar or even lower) would be advantageous and beneficial. For example, some analytes (e.g., cytokines such as IL-17A, IL-12p70, interferon alpha, interferon gamma, IL-1alpha, IL-1beta) have limited detectability in certain sample media (e.g., blood), so quantification requires analytical sensitivity greater than what is conventionally available. As another example, certain complex sample media (e.g., stool, cerebrospinal fluid) may need to be diluted with buffer to reduce matrix effects, which can negatively impact detectability, especially for low-abundance analytes. Improved detectability can also assist with earlier detection of infectious diseases, for example by providing for more sensitive detection of viral and bacterial proteins or other antigens. For certain capture object-based assays such as digital ELISAs, improved sensitivity (e.g., level of detection) increases as the number of detectable species immobilized per capture object is increased. In assays using enzyme labels on beads, such a ratio can be expressed as the average number of enzymes per bead (AEB), and greater AEB can, hypothetically, lead to greater sensitivity. The number of detectable species per capture object (e.g., AEB) for a given sample containing analyte can be increased by decreasing the number of capture objects exposed to the sample. However, using fewer capture objects presents several technical challenges that have discouraged and rendered impractical such an approach. For example, existing capture object-based assay techniques detect capture objects at low efficiencies—typically only 5% of capture objects used to capture analytes from a sample are analyzed. At such low efficiencies, conventional assays would yield an insufficient number of analyzed capture beads, and be considered impractical. Instead, existing techniques either (a) avoid such a problem altogether by instead using a high number of capture objects, or (b) focus on increasing sensitivity solely by increasing the absolute number of capture objects detected instead of the percentage of capture objects detected. The latter approach involves use of a large excesses of capture objects as compared to the number of assay sites (e.g. wells) in an array to increase the fraction of assay sites associated with capture objects (e.g., filling as high a fraction of wells with beads as possible). Certain approaches now described take a contrary approach, instead using relatively fewer capture objects (e.g., fewer than 50,000) in comparison to the number of assay site than in conventional assays, and in some such instances focus on analyzing a high percentage of the capture objects exposed to the sample. Such reduction in the number of capture objects however can conflict with competing considerations. Use of lower numbers of capture objects can lead to increased Poisson noise in digital ELISAs, and can result in slower kinetics and fewer analytes captured in a given time period. Unexpectedly, however, certain methods and equipment described utilize conditions (e.g., sample volume and incubation times) and techniques (e.g., high efficiency capture object immobilization) that can result in increased sensitivity as a result of using fewer capture objects, while avoiding or mitigating at least some or all of the competing complications as described above to a degree to sufficient to provide higher sensitivity as compared to typical existing assay techniques.

Apparatuses and methods for immobilizing capture objects with respect to assay sites are described. Some such methods and apparatuses may facilitate capture object-based assays for detecting and/or quantifying analyte molecules, including assays using relatively few capture objects compared to existing assays.

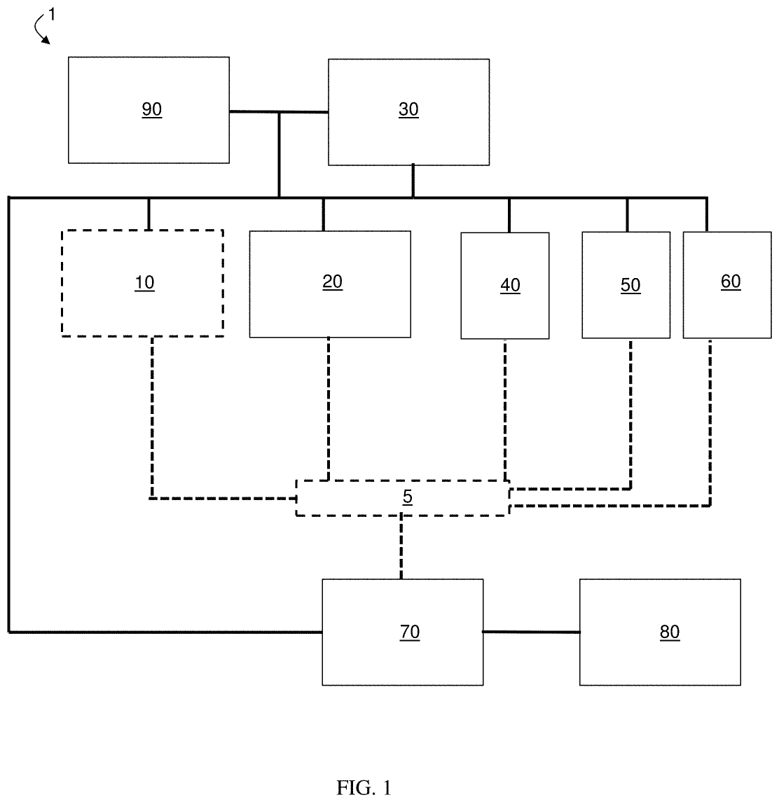

In some instances, an apparatus for immobilizing capture objects with respect to assay sites is described. The apparatus may be a sub-component of larger system comprising an automated apparatus for performing an assay (e.g., for detecting and/or quantifying analyte molecules or particles). FIG. 1 shows an outline of one such non-limiting system 1 including components for immobilizing capture objects. In FIG. 1, system 1 may comprise an optional assay consumable handler 10, which is configured to be operatively coupled to assay consumable 5 (which may be removable and whose presence is optional as indicated by the dashed lines), according to certain embodiments. Such an embodiment may be, for example, an automated robotic system. System 1 may comprise capture object applicator 20, force field generator 40, fluid injector 50, and fluid pump 60. In some embodiments, system 1 comprises one or more controllers 30 comprising one or more processors configured to control and operate certain components of the apparatus. For example, controller 30 may comprise one or more processors configured to control and operate assay consumable handler 10, capture object applicator 20, force field generator 40, fluid injector 50, and fluid pump 60 to perform a method of immobilizing capture objects with respect to assay sites on a surface of assay consumable 5.

In some such instances, controller 30 is configured to modulate fluid pump 60 to move fluid (e.g., in fluid plugs) bi-directionally across a surface of assay consumable 5. It should be understood that in some embodiments a separate assay consumable handler 10 is not required. For example, one or more of the components above may be integrated with the assay consumable (e.g., as part of a microfluidic system on, for example, a chip). Other components of system 1 may be configured to perform other steps or manipulations of an assay. For example, imaging system 70 may comprise a detector and optics for imaging assay sites on the assay consumable, and computer-implemented control system 80 may be configured to receive information from the imaging system and analyze the assay sites (e.g., to determine the presence of capture objects and/or analyte molecules or particles immobilized with respect to the assay sites). System 1 may, in some but not necessarily all instances, further comprise sample washer 90, configured to prepare capture objects and analytes molecules (e.g., from fluid samples) for detection. In other embodiments, such preparation may be performed separately.

Each of the assay consumable handler, capture object applicator, force field generator, fluid injector, and fluid pump may be associated with the same or different controllers (e.g., controller 30) configured to operate the component as described herein. The controller may be configured such that the various stages of the capture object immobilization and/or assay methods are performed automatically. In certain embodiments, one or more components or their functions shown as being separate in FIG. 1 may be integrated into a single component. For example, in certain cases, two or more functions of capture object applicator 20, fluid injector 50, and fluid pump 60 may combined in a single component of the system. As another example, in certain embodiments, a single computer implemented control system (e.g., computer implemented control system 80) may control both operation of imaging system 70 and perform the functions of controller 30 as described above. Therefore, reference to any one of the components does not preclude such component from performing other functions of the system unless specifically so indicated. Similarly, reference to a system comprising separately recited components does not require the components to be physically distinct structural elements unless specifically so illustrated or described as such (e.g., multiple components may share the same structural elements or have structural elements in common but be configured to function as multiple components of the overall system).

### Delivering Capture Objects to Assay Sites Surface

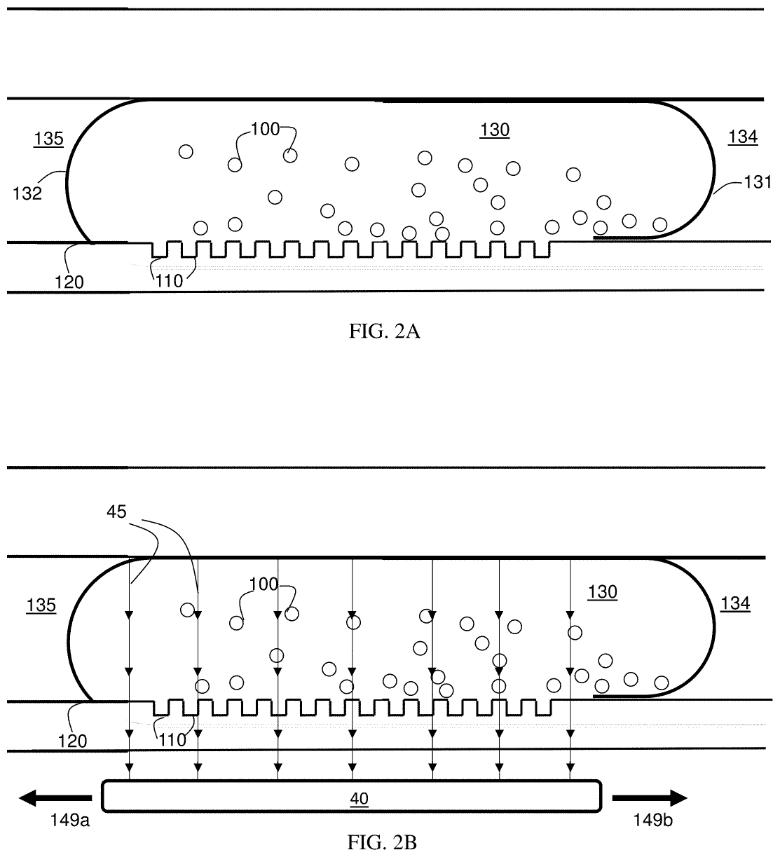

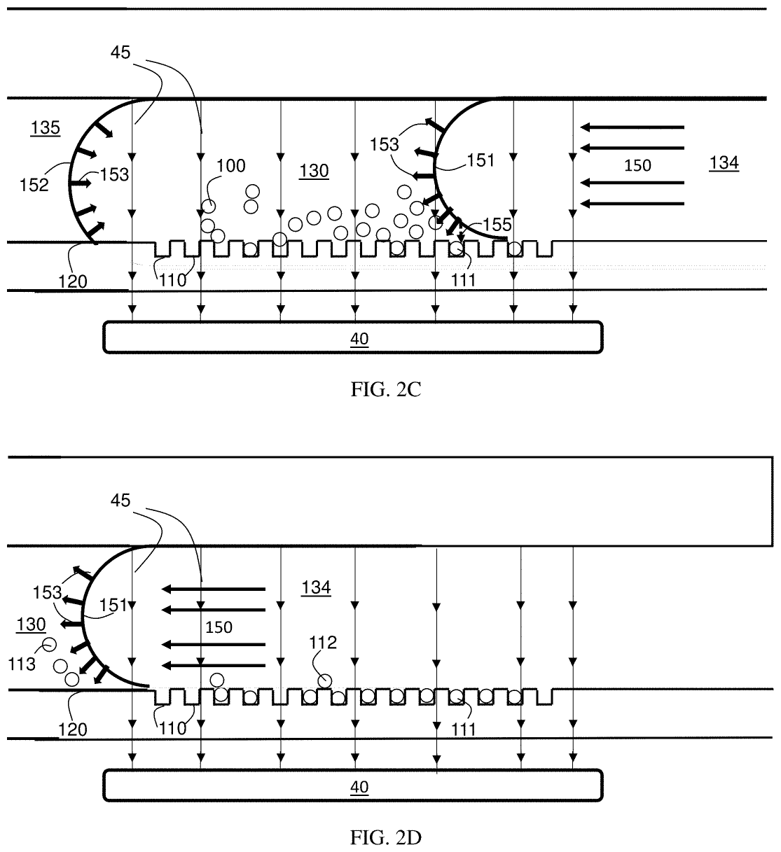

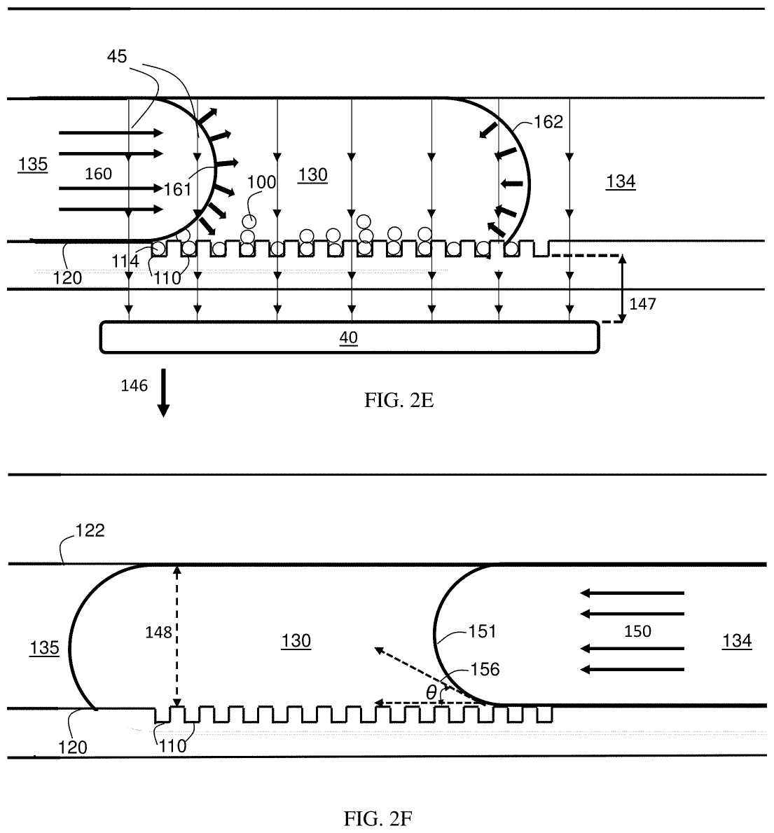

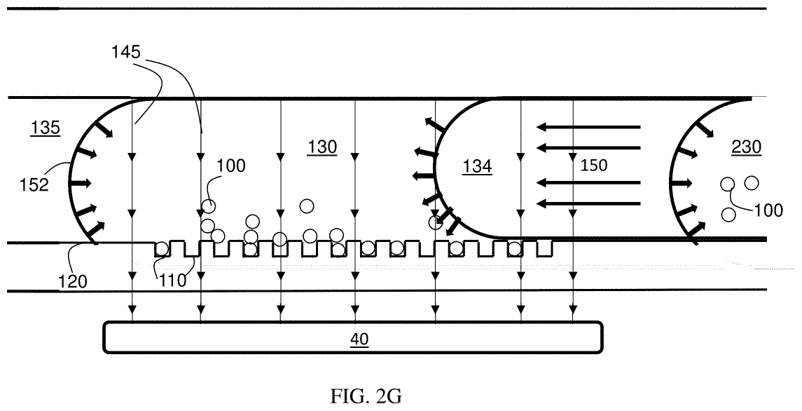

In some embodiments involving immobilization of capture objects, capture objects are delivered in proximity to assay sites on a surface. For example, FIG. 2A depicts a schematic illustration of capture objects 100 delivered in proximity to assays sites 110 on surface 120, in accordance with certain embodiments. While FIG. 2A illustrates capture objects 100 as beads and assay sites 110 as reaction vessels (e.g., wells) in surface 120, other configurations are possible and described in further detail below. In some instances, the capture objects are delivered in proximity to the assay sites via a fluid. The fluid may be in the form of a plug/bolus of any size or volume in which two immiscible phases (at least partially) pass over (at least some of) the assay sites, or alternatively a continuous single phase stream. For example, FIG. 2A depicts delivery of capture objects 100 in fluid plug 130 over assay sites 110, according to certain embodiments.

Capture objects may be delivered in proximity to assay sites to be positioned relatively close to the assay sites (e.g., within 10 mm, within 5 mm, within 1 mm, within 500 micrometers, within 100 micrometers, or less), but need not necessarily be delivered directly into/onto or be immobilized with respect the assay sites immediately upon delivery. The capture objects may be delivered in proximity to the assay sites by any of a variety of techniques, including manually (e.g., by pipetting) or via components of an apparatus such as a capture object applicator described in more detail below.

The delivered capture objects may be subsequently immobilized with respect to the assay sites. For example, capture objects 100 (e.g., beads) may be inserted into assay sites 110. In this context, immobilization of capture objects with respect to assay sites refers to fixing the position of the capture object at the assay site, such as inserting a capture object into a well, encapsulating a capture object within a static droplet, or confining a capture object to a specific area of a surface defining an assay site. Immobilization of a capture object does not necessarily involve attachment of the capture object to the assay site (e.g., chemically, mechanically or otherwise). As mentioned above, efficient rapid immobilization of capture objects can in some instances facilitate the use of smaller numbers of capture objects than certain existing capture object-based technologies.

### Capture Objects

The capture objects may have any of a variety of suitable forms. In some instances, the capture objects are configured to be able to be spatially segregated from each other. The capture objects may be provided in a form allowing them to be spatially separated into a plurality of locations (e.g., assay sites, channels, etc.). For example, the capture objects may comprise beads (which can be of any shape, e.g., sphere-like, disks, rings, cube-like, etc.), a dispersion or suspension of particulates (e.g., a plurality of particles in suspension in a fluid), nanotubes, or the like. In some embodiments, the capture objects are insoluble or substantially insoluble in the solvent(s) or solution(s) utilized in an assay. In some cases, the capture objects are non-porous solids or substantially non-porous solids (e.g., essentially free of pores); however, in some cases, the capture objects are porous or substantially porous, hollow, partially hollow, etc. They may be non-absorbent, substantially non-absorbent, substantially absorbent, or absorbent. In some cases, the capture objects comprise a magnetic material, which may facilitate certain aspect of an assay (e.g., washing step, immobilization/loading step).

The capture objects may be of any suitable size or shape. Non-limiting examples of suitable shapes include spheres, cubes, ellipsoids, tubes, and sheets. In certain embodiments, the average diameter (if substantially spherical) or average maximum cross-sectional dimension (for other shapes) of the capture objects is greater than or equal to 0.1 micrometer, greater than or equal to 1 micrometer, greater than or equal to 10 micrometers, or greater. In some embodiments, the average diameter (if substantially spherical) or average maximum cross-sectional dimension (for other shapes) of the capture objects is less than or equal to 100 micrometers, less than or equal to 50 micrometers, less than or equal to 10 micrometers, or less. Combinations of these ranges are possible. For example, in some embodiments, the average diameter of the capture objects or the maximum dimension of the captures objects in one dimension is between 0.1 micrometer and 100 micrometers, between 1 micrometer and 100 micrometers, between 10 micrometers and 100 micrometers, or between 1 micrometer and 10 micrometers. The “average diameter” or “average maximum cross-sectional dimension” of capture objects is the arithmetic number average of the diameters/maximum cross-sectional dimensions of the capture objects. Those of ordinary skill in the art can determine the average diameter/maximum cross-sectional dimension of a population of capture objects, for example, using laser light scattering, microscopy, sieve analysis, or other known techniques. For example, in some cases, a Coulter counter may be used to determine the average diameter of a plurality of beads.

In certain embodiments, the capture objects are or comprise beads. The beads may be magnetic beads. In some instances in which a magnetic field is generated in proximity to the surface, the magnetic field may act on the magnetic beads and cause the beads to be effectively spatially distributed with respect to the assay sites (e.g., by moving them toward the surface in a desired manner). The magnetic property of the beads may also help in separating the beads from a liquid for example during washing step(s). In some embodiments, the magnetic beads are superparamagnetic, while in some embodiments the magnetic beads are ferromagnetic. As is generally known, superparamagnetic particles are paramagnetic and have a high magnetic susceptibility, while ferromagnetic particles can be magnetized by an external magnetic field and retain magnetized after the external field is removed. Further description of superparamagnetic and ferromagnetic particles in devices is provided in Van Reenen, A., de Jong, A. M., den Toonder, J. M., & Prins, M. W. (2014). Integrated lab-on-chip biosensing systems based on magnetic particle actuation—a comprehensive review. Lab on a Chip, 14(12), 1966-1986, which is incorporated by reference herein in its entirety for all purposes. Potentially suitable beads, including magnetic beads, are available from several commercial suppliers. In some embodiments, at least some of the capture objects delivered in proximity to the surface comprising the assay sites are associated with at least one analyte molecule or particle. In some such embodiments, at least some of the capture objects delivered in proximity to the surface comprising the assay sites are associated with at least one analyte molecule or particle and one or more binding ligands (as described in more detail below).

### Assay Sites

The assay sites may be in any of a variety of suitable forms. As mentioned above and illustrated in FIGS. 2A-2G, the assay sites (e.g., assay sites 110) may be in the form of reaction vessels in a surface (e.g., surface 120). The reaction vessels may be wells (e.g., microwells) in a surface and can be formed using any of a variety of techniques described in more detail below. In some embodiments the assays sites are capable of being fluidically isolated from each other.

For example, the assays sites (e.g., reaction vessels) may comprise continuous peripheral walls such that upon sealing, no fluidic connection exists between the reaction vessels. Other forms of assay sites include, but are not limited to, spatially fixed droplets (e.g., surrounded by an immiscible fluid such as water droplets surrounded by immiscible oil), and hydrophilic regions of a surface surround by hydrophobic regions.

In some embodiments, the assay sites all have approximately the same volume. In other embodiments, the assay sites may have differing volumes. The volume of each individual assay site may be selected to be appropriate to facilitate any particular assay protocol. For example, in one set of embodiments where it is desirable to limit the number of capture objects immobilized with respect to each assay site, the volume of the assay sites may range from attoliters or smaller to nanoliters or larger depending upon the size and shape of the capture objects, the detection technique and equipment employed, the number and density of the assay sites on the surface, and the expected concentration of capture objects delivered to the surface containing the assay sites. In some embodiments, the size of the assay sites (e.g., reaction vessels) may be selected so only a single bead used for analyte capture can be fully contained within the assay site. In some embodiments, the assay sites (e.g., reaction vessels) have a volume of greater than or equal to 10 attoliters, greater than or equal to 50 attoliters, greater than or equal to 100 attoliters, greater than or equal to 500 attoliters, greater than or equal to 1 femtoliter, greater than or equal to 10 femtoliters, greater than or equal to 50 femtoliters, greater than or equal to 100 femtoliters, or greater. In some embodiments, the assay sites have a volume of less than or equal to 100 picoliters, less than or equal to 50 picoliters, less than or equal to 10 picoliters, less than or equal to 1 picoliter, less than or equal to 500 femtoliters, or less. Combinations of these ranges are possible. For example, in some embodiments the assay sites (e.g., reaction vessels) have a volume of greater than or equal to 10 attoliters and less than or equal to 100 picoliters, greater than or equal to 10 attoliters and less than or equal to 50 picoliters, or greater than or equal to 1 femtoliter and less than or equal to 1 picoliter.

In some embodiments, the assay sites are present on the surface as an array. In FIG. 2A, for example, assay sites 110 may be part of an array arranged on surface 120. The assay sites (e.g., reaction vessels) may be arrayed in a regular pattern or may be randomly distributed. In some instances, the array is arranged as a two-dimensional array on the surface (e.g., a substantially planar surface). However, in some embodiments, the assay sites are aligned along a single dimension. As one such example, in some embodiments the assay sites are aligned in a line along a surface of a channel (e.g., a microchannel).

In some embodiments, the assay sites are configured such that immobilized capture objects are arranged on the plane of the surface (e.g., a planar surface of an assay consumable). In some such embodiments, the capture objects arranged on the plane of the surface are arranged as an array. However, in some embodiments, the immobilized capture objects are randomly distributed on the surface (e.g., the planar surface of an assay consumable), with the resulting placement of the immobilized capture objects establishing the locations of the assay sites on the surface. In some such embodiments, force from the force field and/or fluid from a fluid plug can cause and/or accelerate placement of the capture objects on the surface, and force from the force field and/or fluid plug can cause the capture objects to stay in place upon formation of the random distribution on the surface (e.g., for ensuing imaging).

The number of assay sites on the surface may depend on a variety of considerations. In some embodiments where the assays sites (e.g., reaction vessels) are used for capture-object based assays to detect/quantify analytes, the number of assay sites can depend on the number of types of analyte molecules or particles and/or binding ligands employed, the suspected concentration range of the assay, the method of detection, the size of the capture objects, the type of detection entity (e.g., free labeling agent in solution, precipitating labeling agent, etc.). In some embodiments the surface comprises a single assay site (e.g., a single reaction vessel in a channel). However, in some embodiments the surface comprises a large number of assay sites. In some embodiments, the number of assay sites on the surface (in an array or otherwise) is greater than or equal to 1,000, greater than or equal to 10,000, greater than or equal to 100,000, greater than or equal to 200,000, and/or up to 500,000, up to 1,000,000, up to 1,000,000,000, or more.

### Assay Consumable

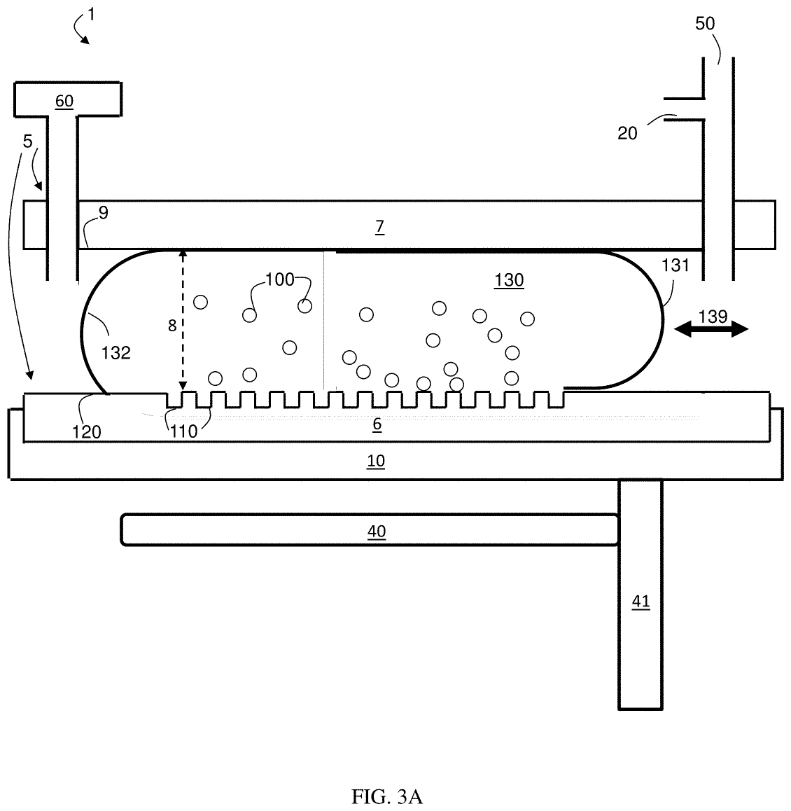

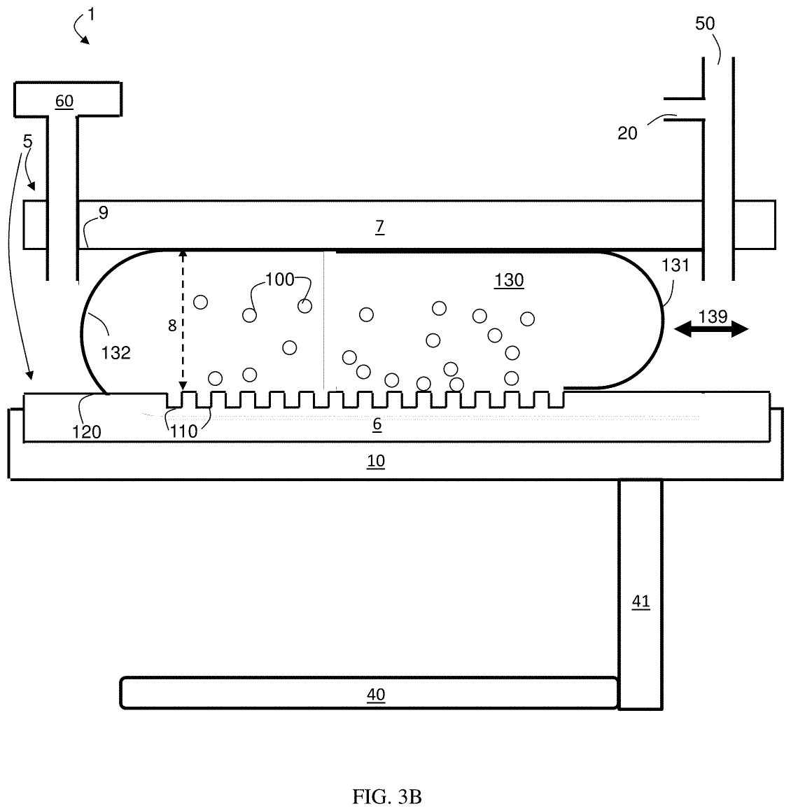

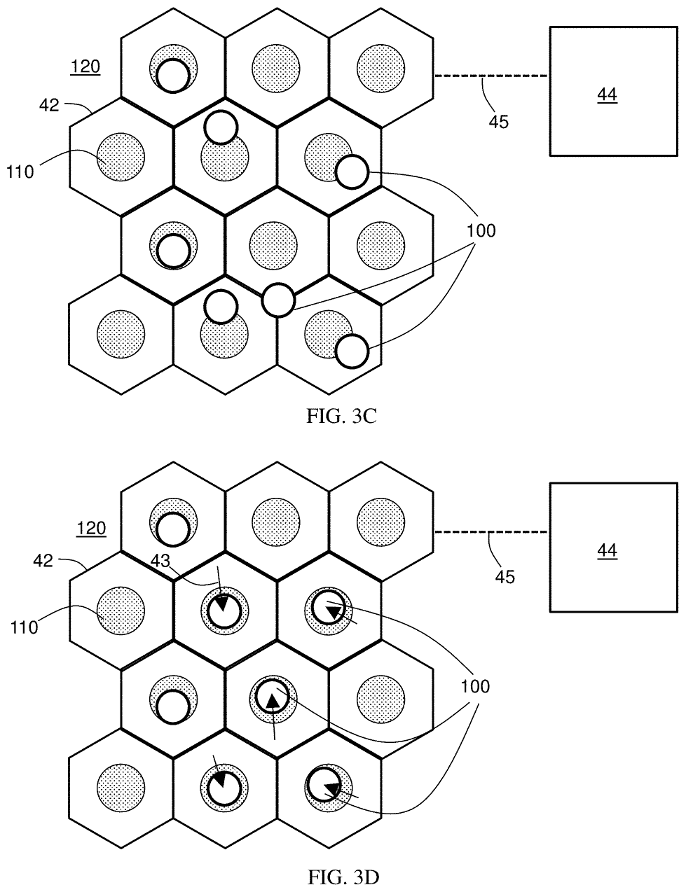

The assay sites described may be part of an assay consumable. FIG. 3A shows a cross-sectional schematic diagram of assay consumable 5 comprising surface 120 comprising assay sites 110, according to one embodiment. While assay consumable 5 shows one set (e.g., array) of assay sites, an assay consumable may comprise more than one set of assay sites, each present in a separate set of spatially separated chambers. For example, the assay consumable having a surface comprising the assay sites (e.g., assay consumable 5) may be in the form of a disk. One such disk is a Simoa™ disk available commercially from Quanterix Corporation. In some cases, the areas surrounding the surface containing assay sites (e.g., reaction vessels/wells) is elevated, so the assay sites/wells are contained in a channel on or in the assay consumable. The channel may be open (e.g., uncovered like a trough) or closed (e.g., enclosed like a tube or conduit). The embodiment illustrated in FIGS. 3A-3B depict assay consumable 5 having a closed channel defined by lower portion 6 and upper portion 7, the channel having height 8 at assay sites 110 (defined as a distance between surface 120 and upper surface portion 9 of assay consumable 5). Examples of suitable assay consumables having surfaces comprising assay sites are described in U.S. patent application Ser. No. 13/035,472, filed Feb. 25, 2011, published as US 2012-0196774, entitled “SYSTEMS, DEVICES, AND METHODS FOR ULTRA-SENSITIVE DETECTION OF MOLECULES OR PARTICLES,” by Fournier et al.), which is incorporate by reference herein for all purposes.

In some embodiments, the total number of capture objects delivered in proximity to the assay sites is less than or equal to the number of assay sites. For example, referring to FIG. 2A, the number of delivered capture objects 100 is less than or equal to the number of assay sites 110 on surface 120. While typical existing techniques for immobilizing capture objects (e.g., for capture-object based assays such as digital ELISAs) employ a large excess of capture objects relative to the number of assay sites (e.g., by a factor of 2, factor of 5, or more), certain embodiments herein take a contrary approach. As described in more detail below, use of a small number of capture objects may, counter-intuitively, improve assay sensitivity provided a sufficient number are detected. In some embodiments, the total number of capture objects delivered in proximity to the reaction vessels is less than or equal to 100,000, less than or equal to 50,000, less than or equal to 25,000, less than or equal to 10,000, less than or equal to 5,000, less than or equal to 2,000, or lower. In some embodiments, a single capture object is delivered in proximity to the assay sites (or a single assay site). However, in some embodiments, the total number of capture objects delivered in proximity to the assay sites is greater than or equal to 100, greater than or equal to 200, greater than or equal to 500, greater than or equal to 1,000, or greater. Combinations of these ranges are possible. For example, in some embodiments the total number of capture objects delivered in proximity to the assay sites is greater than or equal to 100 and less than or equal to 100,000, or greater than or equal to 1,000 and less than or equal to

As mentioned above, in some embodiments, the total number of capture objects delivered in proximity to the assay sites is less than or equal to the number of assay sites. In some embodiments, a ratio of the total number of capture objects delivered in proximity to the assay sites to the number of assay sites is less than or equal to 1:1, less than or equal to 1:2, less than or equal to 1:3, less than or equal to 1:4, less than or equal to 1:5, less than or equal to 1:10, less than or equal to 1:20, less than or equal to 1:30, less than or equal to 1:40, and/or as low as 1:50, as low as 1:100, as low as 1:1,000, as low as 1:2,000, or lower.

### Generating Force Field/Force Field Generator

In some embodiments, an external force is applied to a capture object delivered in proximity to an assay site on the surface. In some such embodiments, a force field is generated in proximity to the surface comprising the assay site or assay sites. In some instances, the force field is generated by a force field generator. As mentioned above, apparatus 1 may comprise force field generator 40 (as shown in FIGS. 1 and 3A-3B). FIG. 2B shows one such embodiment, where force field generator 40 generates a force field represented by vector field 45, according to certain embodiments. The force field proximate the surface may act on the capture objects delivered in proximity to the assay sites so the capture objects move toward the surface. For example, in FIG. 2B, the force field represented by vector field 45 may act on capture objects 100 so capture objects 100 move in a direction parallel to the arrows of vector field 45 toward surface 120. In some embodiments, the force field is a magnetic field. For example, in FIG. 2B, capture objects 100 may be magnetic (e.g., magnetic beads) and a magnetic field represented by magnetic vector field 45 acts on capture objects 100. As another example, the force field may be an electric field and the capture objects may have an electrostatic charge (e.g., due to functionalization of the capture object with charged moieties). In such a case, an applied electric field with vector lines pointing away from the surface would move capture objects carrying a negative charge toward the surface, and an applied electric field with vector lines pointing toward the surface would move capture objects carrying a positive charge toward the surface.

Applying a force from a force field on the capture objects in a direction with a component toward the surface comprising the assay sites may rapidly decrease the distance between the capture objects and the assay sites. In doing so, the time required for immobilizing the capture objects with respect to the assay sites may be decreased. Additionally, the force field acting on the capture objects may assist with keeping the capture objects in place and reduce the extent to which other forces (e.g., fluid dynamic forces, sealing steps) move the capture objects away from the surface and assay sites. Additionally, it has been discovered that generation of such a force field may have a synergistic effect with one or more other techniques described in this disclosure, including those relating to fluid flow.

### Magnetic Field

As mentioned above, in some embodiments the force field generated in proximity to the surface comprising the assay sites is a magnetic field. The magnetic field may be generated according to techniques known in the art. For example, the force field generator may comprise a permanent magnet and/or an electromagnet. A permanent magnet may comprise any of a variety of materials known in the art, such as ferromagnetic or ferromagnetic materials. A permanent magnet may comprise transition metals (e.g., iron, cobalt, nickel, titanium) and alloys thereof and/or rare earth metals (e.g., neodymium, samarium) and alloys thereof. Electromagnets generally generate magnetic fields via passing electrical current through a coil (e.g., solenoid). An electromagnet may comprise a coil of electrically conductive material (e.g., copper, silver) around a ferromagnetic or ferromagnetic core (e.g., iron). In FIG. 2B, force field generator 40 can be a permanent magnet and/or an electromagnet below surface 120 comprising assay sites 110. Such a configuration in which the assay sites are between the force field generator (e.g., magnet) and the delivered capture objects is illustrative, as other configurations are possible. For example, in some embodiments force field generator 40 may be above fluid 130 containing capture objects 100 and the generated force field represented by vector field 45 may act repulsively on the capture objects with respect to the force field generator so capture objects 100 move toward surface 120 comprising assay sites 110. In some instances, the magnetic field is generated so a magnetic field vector of the magnetic field is directed from the surface towards a bottom of the assay sites. For example, vector field 45 in FIG. 2B, in the form of a magnetic vector field, may point in a direction from surface 120 toward a bottom of assay sites 110. Such a configuration of a magnetic vector field may act on capture objects 100 so they move toward the bottom of assay sites 110, according to some embodiments.

The magnitude of the magnetic field may depend on the position of the force field generator (e.g., permanent magnet, electromagnet). In some embodiments, an apparatus is configured to position a permanent magnet and/or an electromagnet below the assay sites of the assay consumable so the permanent magnet and/or electromagnet can generate a magnetic field at the surface of the assay consumable at a desired magnitude. In some embodiments, the magnitude of the magnetic field at the surface of the assay consumable is between 0.1 and 2 Tesla or between 0.2 and 1 Tesla. It has been observed that certain magnet positions relative to the assay consumable (and certain magnetic field strengths and radial vs. axial distributions) may result in advantageous delivery of capture objects to the surface comprising the assay sites. For example, placing a force field generator (e.g., permanent magnet) too close to the bottom of the assay sites may result in a magnetic field that causes capture objects (e.g., magnetic beads) to pellet toward edges of a collection of assay sites on the surface. However, placing a force field generator (e.g., permanent magnet) too far from the bottom of the assay sites may result in a magnetic field that causes the beads to pellet toward the center of a collection of assay sites. In some embodiments, the apparatus is configured to position the force field generator between 0 mm and 5 mm from a bottom of the assay sites of the assay consumable.

### Electric Field

In some embodiments, the force field generated in proximity to the surface comprising the assay sites is an electric field. The electric field may be generated according to techniques known in the art. For example, the force field generator 40 may comprise one or more electrically conductive solids coupled to an electrical circuit in proximity to the surface 120. As a specific example, the force field generator 40 may be configured as a capacitor with a first electrically conductive article (e.g., a first metal layer or plate) coupled to an electrical circuit positioned below the assay sites 10 and the surface 120 and a second electrically conductive article (not shown) electrically coupled to the circuit (e.g., a second metal layer or plate) positioned above the assay sites and parallel to the first electrically conductive article. Application of a voltage to the electrical circuit may cause an electrical field with vector components toward and normal to the surface comprising the assay sites acting on the capture objects (if carrying an electrical charge) so the capture objects move toward the surface, as described above.

### Dielectrophoresis

In some embodiments, the force field generated in proximity to the surface comprising the assay sites is a non-uniform electric field. A non-uniform electric field may cause a dielectrophoretic force that acts on the capture objects delivered in proximity to the assay sites (e.g., near, at, onto, and/or into the assay sites) so that the capture objects move toward the surface and/or along a plane of the surface. Dielectrophoresis refers to the phenomenon where a polarizable dielectric particle (which may be utilized as a capture object) experiences a force when subject to a non-uniform electric field, with the magnitude and sign of the force (e.g., repulsive or attractive with respect to an electric field gradient) depending on a variety of factors including the medium and particle's electrical properties, the size and shape of the particle, and the frequency of the electric field (in instances where the non-uniform electric field is produced using an alternating current having that frequency). A particle need not carry an electrostatic charge to experience a dielectrophoretic force. In certain embodiments, dielectrophoretic methods can be used to promote immobilization of capture objects (e.g., beads) with respect to assay sites on the surface using attractive and/or repulsive forces from non-uniform electric fields. The non-uniform field can be an alternating current (AC) electric field or a direct current (DC) electric field. Theory and implementation of dielectrophoresis in microfluidic applications are described in Pethig R. “Review article dielectrophoresis: Status of the theory.” Biomicrofluidics. 2010; 4(2):022811, and in Pesch G R, et al., “A review of dielectrophoretic separation and classification of non-biological particles.” Electrophoresis. 2021 January; 42(1-2):134-52, each of which is incorporated herein by reference in its entirety for all purposes. As mentioned above, in some embodiments, the force field generator 40 comprises one or more electrically conductive solids coupled to an electrical circuit in proximity to the surface 120. A non-uniform electric field may be generated from the electrically conductive solids (e.g., electrodes) coupled to an electrical circuit in proximity to the surface 120 to create a non-uniform electric field at a frequency selected so that the capture objects move toward the surface, for example a surface comprising assay sites (and in some instances toward the bottom of a reaction vessel when such assay sites are used) or towards a unfeatured surface to form randomly distributed assay sites comprising the capture objects.

In some embodiments, negative dielectrophoresis is employed, where a repulsive effect from the electric field causes polarizable dielectric capture objects (including uncharged capture objects) to move toward assay sites on a surface (e.g., toward a surface comprising the assay sites and/or along the surface toward the assay sites). In some such embodiments, the electrically conductive solids of the force field generator are located opposite the surface so that capture objects delivered between the electrically conductive solids and the surface are repelled from the electrically conductive solids and therefore towards the surface, e.g., a surface comprising assay sites (e.g., reaction vessels). In some embodiments where the surface comprising assay sites is part of a closed channel (e.g., a microfluidic channel), the electrically-conductive solids that repel the capture objects via negative dielectrophoresis are located adjacent to a portion of the channel opposite the assay sites. As mentioned above, negative dielectrophoresis can be employed by using an appropriately selected frequency for the electric field, which can be screened facilely by testing various fields in the presence of the capture objects until a repulsive effect is observed. In some embodiments where negative dielectrophoresis is employed, at least some of the electrically conductive solids (e.g., electrodes) are adjacent (e.g., directly adjacent) to a surface, for example a surface comprising assay sites.

Some such electrically conductive solids may form a network of electrodes (e.g., as wires) on the surface surrounding at least some of the assay sites on a surface. For example, in some embodiments the assay sites are reaction vessels in the surface and at least some of the area of the surface surrounding the reaction vessels comprises electrically conductive solid in conductive or inductive electrical communication with the force field generator (e.g., with a power source). FIG. 3C shows a top view schematic illustration of one such embodiment where surface 120 comprises assay sites 110 in the form of reaction vessels (e.g., microwells) surrounded by a network of electrically conductive solids 42 in the form of wire electrodes adjacent to surface 120 that are in conductive or inductive electrical communication with power source 44 via electrical connection 45, and capture objects 100 in the form of polarizable dielectric beads are in proximity of assay sites 110. A repulsive force from such electrically conductive solids adjacent to the surface may cause polarizable dielectric capture objects, e.g., in the form of beads, located on the surface but not inserted into the reaction vessels to move along the surface toward the reaction vessels (which do not repel the capture objects). For example, FIG. 3D shows capture objects 100 moving in directions represented by arrows 43 along surface 120 toward assay sites 110 due to repulsive dielectrophoretic forces from network of electrically conductive solids 42 (e.g., upon formation of a non-uniform electric field by application of alternative current through electrically conductive solids 42), resulting in insertion of capture objects 100 into assay sites 110. In such a way, immobilization of the capture objects via insertion into the reaction vessels may be accelerated via a dielectrophoretic repulsive force toward the surface and/or along the surface.

In some embodiments, positive dielectrophoresis is employed, where an attractive effect from the electric field causes the capture objects (including uncharged capture objects) to move toward the surface, which may comprise assay sites (e.g., toward a surface comprising the assay sites and/or along the surface toward the assay sites). In some such embodiments, the electrically conductive solids in conductive or inductive electrical communication with the force field generator (e.g., with a power source) are located adjacent (e.g., directly adjacent) to a surface comprising assay sites so that capture objects delivered in proximity to the assay sites and attracted to the electrically conductive solids move toward the surface comprising the assay sites (e.g., reaction vessels). As mentioned above, positive dielectrophoresis can be employed by using an appropriately selected frequency for the electric field, which can be screened facilely by testing various fields in the presence of the capture objects until an attractive effect is observed. In some embodiments where positive dielectrophoresis is employed, at least some of the electrically conductive solids (e.g., electrodes) are adjacent (e.g., directly adjacent) to the bottoms of assay sites on a surface. For example, in some embodiments the assay sites are reaction vessels in the surface and at least some of the areas of the bottom surfaces of the reaction vessels (e.g., bottom surfaces of microwells) comprise electrically conductive solid in conductive or inductive electrical communication with the force field generator (e.g., with a power source). An attractive force from such electrically conductive solids at the bottoms of the assay sites may cause capture objects in proximity to the reaction vessels, including capture objects located on the surface but not inserted into the reaction vessels, to move toward and/or along the surface toward the reaction vessels. In such a way, immobilization of the capture objects via insertion into the reaction vessels may be accelerated via a dielectrophoretic attractive force toward the surface and/or along the surface.

While some embodiments in this disclosure for immobilizing capture objects with respect to assay sites involve a sequential or concurrent combination of a force field from a force field generator (e.g., a magnetic field, an electric field) and force from a receding meniscus of a fluid plug to promote the immobilization, other embodiments can involve promoting association of the capture objects via application of force primarily (or completely) from the externally applied force field from a force field generator. In some embodiments, for example, where digital microfluidics is employed for delivering the capture objects in proximity to assay sites on a surface (as opposed to, for example, substantially continuous flow as described in more detail below), a magnitude of force contributed from a receding meniscus of the fluid plug may be relatively small and may not point in a direction promoting delivery of the capture objects to the assay sites. In some such embodiments, the externally applied force field from a force field generator may provide the primary or sole contribution to facilitating delivery of the capture objects to the assay sites without substantial additional contribution from forces generated by a receding meniscus, such that in some such cases generating a first direction receding meniscus and a second direction receding meniscus would not be required. For example, in some embodiments a capture object can be associated with an assay site on a surface by: flowing a fluid plug containing the capture object to the assay site (e.g., to contact and wet the assay site) using digital microfluidics techniques (e.g., electrowetting on dielectric and/or electrophoresis techniques); generating a non-uniform electric field to apply an external dielectrophoretic force to the capture object subjected to the delivering step such that a distance between the capture object and the assay site is decreased; and associating the capture object with respect to the assay site via application of a force contributed at least in part by the dielectrophoretic force. In some embodiments, an apparatus is provided for associating capture objects with assay sites on a surface of an assay consumable wherein the force field generator comprises a power source and electrically conductive solids (e.g., electrodes) in conductive or inductive electrical communication with the power source that are adjacent to or opposite a surface of the consumable, and the apparatus comprises a controller comprising one or more processors configured to initiate application of a voltage to at least some of the electrically conductive solids by the power source to generate an electric field that moves the fluid plug across at least part of a surface of the assay consumable (e.g., to one or more assay sites). The electrically conductive solids that generate the electric field that moves the fluid plug may be adjacent to the surface of the assay consumable (e.g., under a dielectric layer). The one or more processors may be configured to send a signal to the power source to apply a voltage to at least some of the electrically conductive solids and then, at a later time, send a signal to the power source to apply a similar or different voltage to different electrically conductive solids. In some embodiments, the one or more processors are configured to initiate application of a voltage to at least some of the electrically conductive solids by the power source to generate a non-uniform electric field in proximity to the surface capable of applying a dielectrophoretic force to polarizable dielectric capture objects. For example, the one or more processors may be configured to send a signal to the power source to apply a voltage causing an alternating current at a frequency the causes dielectrophoresis. Some such electrically conductive solids generating the non-uniform electric field may be the same as those used to cause movement of the fluid plug across at least part of the surface (e.g., via a digital microfluidics process). However, in other embodiments, the one or more processors are configured to initiate application of a voltage to at least some of the electrically conductive solids by the power source to generate a non-uniform electric field using some of the electrically conductive solids (e.g., adjacent or opposite the surface), and other of the electrically conductive solids (e.g., adjacent to the surface) receiving a voltage from the power source are used to move the fluid plug across at least part of the surface (e.g., using digital microfluidics such as electrowetting on dielectric techniques). In some such embodiments, voltage applied to the electrically conductive solids that cause the non-uniform electric field capable of applying the dielectrophoretic force is of a different magnitude and/or is applied at a different time than voltage applied to the electrically conductive solids that cause movement of the fluid plug. The electrically conductive solids (e.g., electrodes) that generate the non-uniform electric field may be in conductive or inductive electrical communication with a same power supply of the power source as the electrically conductive solids that induce movement of the fluid plug across at least a part of the surface, or a different power supply.

The force field generator may be a component of the apparatus for immobilizing the capture objects. The force field generator may be adjacent to the assay consumable when operatively coupled to the assay consumable handler. It should be understood when a first object is adjacent a second object, one or more intervening objects may be present between the first object and the second object. In some embodiments, the force field generator is directly adjacent to the assay consumable when operatively coupled to the assay consumable handler, such that no intervening components are between the force field generator and the assay consumable. Referring again to FIG. 3A, apparatus 1 may comprise force field generator 40, and apparatus 1 may have at least one configuration in which force field generator 40 is adjacent and below assay consumable 5 when the assay consumable is present (e.g., operatively coupled with assay consumable handler 10). In some such embodiments, force field generator 40 of apparatus 1 comprises a magnet (e.g., permanent magnet, electromagnet). In some such instances, the force field generator (e.g., force field generator 40) is configured to generate a magnetic field generating a magnetic field vector of the magnetic field directed from the surface towards a bottom of the assay sites (e.g., assay sites 110).

### Sequence of Field Generation

Generation of the force field (e.g., magnetic field, electric field) in proximity to the surface comprising the assay sites may occur at any of a variety of times during the performance of the methods described. In some embodiments the force field is generated before delivering the capture objects, while in certain embodiments the force field is generated during delivery of the capture objects, and in some embodiments the force field is generated following delivery of the capture objects to the surface. To illustrate, while FIG. 2A shows delivered capture objects 100 in fluid 130 in proximity to assay sites 110 in the absence of a force field, in some embodiments a force field represented by vector field 45 may be present before delivery of capture objects.

### Fluid Plug Flow

In some embodiments, the delivered capture objects are contained within a fluid plug. For example, delivered capture objects 100 in FIG. 2A may be contained within a fluid plug 130. A fluid plug (or equivalently a bolus) as used in this disclosure is an isolated volume of the fluid in at least partial contact with an immiscible phase (e.g., a gas phase or immiscible liquid phase) other than the solid channel wall(s) or other solid surfaces with which it is in contact. A fluid plug is not limited by any particular volume or shape. For example, while some fluid plugs relative to the channel dimensions in which they are contained and flow may be relatively small (e.g., less than or equal to 3 μL or less in certain of the fluidic systems suitable for certain embodiments in this disclosure), other fluid plugs may be larger (e.g., greater than or equal to 15 μL, greater than or equal to 30 μL, or more). Some fluid plugs may have a shape substantially conforming to the cross-sectional shape of the channel in which they are flowing (e.g., with a circular, square, or rectangular cross-section) (notwithstanding menisci described below) under some conditions (e.g., during flow through a channel having a circular, square, or rectangular cross-sectional shape). However, some fluid plugs may have substantially non-cylindrical shapes over at least a portion of their length along their direction of flow and in general may have shapes depending on and conforming to, for example the channel shape and configuration in which the flow (e.g., due to partial passage though channel turns or intersections, changes in channel shape or dimensions along the length of flow, etc.). Some fluid plugs passing through a channel may have a length in the channel direction substantially greater than a cross-sectional dimension of the channel (e.g., by a factor of 2, factor of 3, factor of 5, factor of 10, or more) and may have a length that is any desired fraction of the total channel length, or even greater in some cases. While in some embodiments the capture objects are delivered in proximity to the assay sites by flowing the fluid plug containing the capture objects at least partially across the assay sites, in other embodiments the capture objects may be delivered in proximity to the assay sites separately from the fluid plug. For example, the capture objects may be deposited in proximity to the surface in a different fluid or in the absence of fluid, following by a step of injecting fluid in proximity to the surface to form the fluid plug.

In some embodiments, a fluid plug containing the capture objects is flowed in a first direction. For example, FIG. 2C shows a schematic illustration where fluid plug 130 flows in first direction 150. Flowing a fluid plug containing the capture objects delivered in proximity to the assay sites may contribute to the immobilization of the capture objects with respect to the assay sites (e.g., inserting beads into reaction vessels), as described in more detail below. Fluid plugs are generally separated from solid objects and/or immiscible fluids by one or more interfaces. Interfaces between a fluid plug and an immiscible fluid surrounding the fluid plug may form menisci, the shape of which may depend on surface tension effects determined by the composition of the fluid plug, the immiscible fluid, and/or any solid surface in contact with the fluid plug. In some embodiments, the fluid plug comprises a first meniscus and a second meniscus, each of which is adjacent to an immiscible fluid. Referring to FIG. 2A, for example fluid plug 130 may have first meniscus 131 adjacent to first immiscible fluid 134 and have second meniscus 132 adjacent to second immiscible fluid 135.

In some embodiments, the fluid plug containing the capture objects comprises a liquid. For example, the fluid plug may comprise water (e.g., as solvent of an aqueous solution such as a buffer solution). In some embodiments, the fluid plug comprises a solution comprising one or more reagents (e.g., a substrate that can react with binding ligands that may be associated with at least some of the capture objects). In certain instances, the fluid plug comprises an organic liquid (e.g., N,N-dimethylformamide (DMF), dimethyl sulfoxide (DMSO), alcohols such as ethanol or 2-propanol). Any of a variety of immiscible fluids may be used in conjunction with the fluid plug. In some embodiments, the immiscible fluid (e.g., immiscible fluid 134 or immiscible fluid 135) is or comprises a gas. Exemplary gases include inert gases (e.g., nitrogen gas, argon) non-inert gases (e.g., oxygen gas), or mixtures thereof (e.g., ambient air). In some embodiments, the immiscible fluids comprise liquid immiscible with the fluid of the fluid plug. As one example, in some embodiments where the fluid plug comprises water (e.g., an aqueous solution), one or more immiscible fluid adjacent the fluid plug comprises an oil (e.g., hydrofluoroether oils).

In some embodiments, a fluid plug is introduced to the surface comprising the assay sites (e.g., on an assay consumable) via a fluid injector. For example, apparatus 1 may comprise fluid injector 50 configured to generate a fluid plug having a first meniscus and a second meniscus each adjacent to an immiscible fluid (e.g., a gas) when on the surface of the assay consumable. In some instances the fluid injector is coupled to a channel of an assay consumable comprising the surface comprising the assay sites. FIGS. 3A-3B, for example, shows fluid injector 50 fluidically coupled to assay consumable 5 when operatively coupled to assay consumable handler 10, and fluid injector 50 may further be fluidically coupled to a fluid pump and a fluid source (e.g., a source of sample or reagent fluid). Fluid injector 50 may inject fluid plug 130 comprising first meniscus 131 and second meniscus 132. Pump 60 may cause fluid plug 130 to flow across surface 120 of assay consumable 5. For example, in some embodiments, Pump 60 is an air or vacuum pump positioned on a distal side of fluid plug 130 with respect to fluid injector 20/50 (as shown in FIG. 3A), and Pump 60 is configured to provide a source of pressurized air and/or vacuum generating a pressure differential that causes fluid plug 130 to flow across surface 120 (e.g., at least partially across assay sites 110). In alternative embodiments fluid pump 60 may pump a liquid that is immiscible with fluid plug 30. In certain embodiments, fluid pump 60 may be fluidically connected to the fluid injector, e.g. via a switchable/controllable fluidic connection to port 20, to facilitate bi-directional fluid motion of fluid plug 130 by selectively and alternately applying a pressure/vacuum to the inlet of fluid pump 60 to the fluid channel (left of plug 130 as illustrated in FIG. 3A) and to the inlet of fluid injector 50 to the fluid channel (right of plug 130 as illustrated in FIG. 3A).

Flowing the fluid plug containing the capture objects in a first direction can create a first direction advancing meniscus and a first direction receding meniscus. Referring to FIG. 2C, for example, flowing fluid plug 130 containing capture objects 100 in first direction 150 (defined by arrows pointing right to left) defines first direction advancing meniscus 152 adjacent immiscible fluid 135 (e.g., air) and receding meniscus 151 adjacent immiscible fluid 134 (e.g., air). In FIG. 3A, fluid plug 130 flows to the left toward fluid pump 60 (e.g., upon application of a vacuum by fluid pump 60), first meniscus 131 becomes a receding meniscus and second meniscus 132 becomes an advancing meniscus.

In some embodiments, the fluid plug is flowed in the first direction so the first direction receding meniscus flows across at least some of the assay sites on the surface. One example of this is illustrated in FIG. 2C, where first direction receding meniscus 151 flows over at least some of assay sites 110. Flowing a receding meniscus of a fluid plug across at least some assay sites may promote immobilization of capture objects in the fluid plug with respect to the assay sites. As a specific example, flowing a receding meniscus of a fluid plug comprising beads across reaction vessels (e.g., wells) on a surface may facilitate insertion of the beads into the reaction vessels. It has been discovered in the context of the present disclosure that certain operating and dimensional parameters of such flow may contribute to relatively efficient and effective immobilization. Some such embodiments involve configuring the flow to create a meniscus able to apply a force on the capture objects with a component pointing toward and normal to the surface (e.g., toward the bottom of reaction vessels). In some embodiments, the first direction receding meniscus is flowed across at least 10%, at least 25%, at least 50%, at least 75%, at least 90%, at least 95%, or more of the assay sites during the step of flowing the fluid plug in the first direction. In some embodiments, the first direction receding meniscus is flowed across an entirety of the assay sites (e.g., so an entirety of the fluid plug is flowed past the assay sites on the surface). For example, FIG. 2D shows an entirety of fluid plug 130, including first direction receding meniscus 151, having flowed past assay sites 110 of surface 120. Some of the capture objects may be immobilized with respect to the assay sites after the first direction receding meniscus has flowed across at least some (or all) of the assay sites, while some of the capture objects may remain in proximity to the assay sites while remaining non-immobilized, and while yet other capture objects may remain in the fluid plug. Referring back to FIG. 2D for example, capture object 111 is immobilized with respect an assay site 110, while capture object 112 remains in proximity to an assay site 110 while not being immobilized with respect to it, and while capture object 113 remains contained in fluid plug 130 even after an entirety of fluid plug 130 has flowed across assay sites 110.

While in some embodiments the fluid plug containing the capture objects is flowed across at least some of the assay sites on the surface a single time, in certain embodiments the fluid plug is flowed across the assay sites multiple times. Some such embodiments may involve reversing the flow direction of the fluid plug. It has been observed in the context of the present disclosure that flowing the fluid plug (e.g., including its receding meniscus) across the assay sites multiple times can lead to unexpectedly efficient immobilization of capture objects with respect to the assay sites. In some embodiments, the fluid plug is flowed in a second, different direction (relative to the first direction). In some instances, the second direction is a reverse direction with respect to the first direction (e.g., different by an angle of 180 degrees). For example, FIG. 2E shows fluid plug 130 being flowed in second direction 160 that is a reverse of first direction 150 shown in FIG. 2C. Flowing the fluid plug in a second direction defines a second direction advancing meniscus and a second direction receding meniscus. The embodiment shown in FIG. 2E, for example, shows fluid plug 130 flowing with second direction advancing meniscus 162 and second direction receding meniscus 161. In some instances, the fluid interface defining the first direction receding meniscus is the same as that defining the second direction advancing meniscus, and the fluid interface defining the first direction advancing meniscus is the same as that defining the second direction receding meniscus.

In some embodiments, the fluid plug is flowed in the second direction so the second direction receding meniscus flows across at least some of the assay sites on the surface. Referring again to FIG. 2E, second direction receding meniscus 161 flows over at least some of assay sites 110. Such flow may result in further immobilization of capture objects with respect to the assay sites. For example, referring again to FIG. 2E, upon flow of fluid plug 130 in second direction 160, second direction receding meniscus 161 may contribute to immobilization of capture object 114 in one of the assay sites 110. It has been discovered in the context of this disclosure that a second “meniscus sweep” over some or all of the assay sites can, in some instances, efficiently and rapidly immobilize capture objects with respect to the assay sites, especially capture objects not immobilized during the flow of the first direction receding meniscus. In embodiments where certain dimensional and operational parameters result in a receding meniscus applying a force on the capture objects toward and normal to the surface, such multiple sweeps of fluid plug menisci across some or all of the assay sites can lead to immobilization of an unexpectedly high number of capture objects compared to simple flow or even simple bi-directional flow methods. In some embodiments, the second direction receding meniscus is flowed across at least 10%, at least 25%, at least 50%, at least 75%, at least 90%, at least 95%, or more of the assay sites during the step of flowing the fluid plug in the second direction. In some embodiments, the second direction receding meniscus is flowed across an entirety of the assay sites (e.g., such that an entirety of the fluid plug is flowed completely past the assay sites on the surface).

One way a fluid plug may be flowed across the surface in the manner described is via a fluid pump. In some embodiments, an apparatus described (e.g., apparatus 1) comprises a fluid pump capable of moving fluid across at least part of the surface, and a controller comprising one or more processors configured to modulate the fluid pump to move the fluid plug bi-directionally across at least part of the surface. Referring to FIGS. 3A-3B, for example, apparatus 1 may comprise fluid pump 60 in fluid communication with assay consumable 5 when operatively coupled to assay consumable handler 10, and fluid pump 60 may be configured to move fluid plug 130 across surface 120 of lower portion 6 of assay consumable 5 in a bi-directional manner as represented by double-arrows 139. The fluid pump may accomplish bi-directional flow of the fluid across the surface in any of a variety of ways. For example, the fluid pump may be configured to alternate between applying a positive pressure differential to the fluid plug (e.g., by pressurizing a gas behind the fluid plug) and applying a negative pressure differential (e.g., by applying a vacuum). Modulation of fluid pump 60 may be controlled by one or more controllers (e.g., controller 30). The controller may, for example comprise one or more processors programmed to provide an appropriate sequence of actuation signals to the fluid pump, or one or more processors capable of receiving input signals from a user indicating that actuation of the fluid pump should occur. In some embodiments, one or more processors can modulate the fluid pump to flow a fluid plug in a first direction so a first direction receding meniscus of the fluid plug flows across a portion or all of the reaction vessels, and flow the fluid plug in a second, different direction so a second direction receding meniscus of the fluid plug flows across a portion or all of the reaction vessels. In some embodiments, the controller comprises one or more processors that can modulate the fluid pump to flow a fluid bi-directionally by being programmed to actuate a fluid pump to provide a positive pressure and then, at a later time, actuate a fluid pump to provide a negative pressure. Alternatively, in some embodiments, the controller comprises one or more processors that can modulate the fluid pump to flow a fluid bi-directionally by being programmed to actuate a fluid pump to apply a positive (or negative pressure) in a first direction and then, at a later time, actuate a fluid pump to apply a positive (or negative) pressure in a second, different direction.

### Meniscus Forces and Parameters

As mentioned above, certain operational (e.g., flow pattern, flow rate, contact angle) and dimensional (e.g., fluid plug volume, channel size) parameters have been determined in the context of the present disclosure to influence immobilization of capture objects via fluid plug techniques. In some instances, the method may be performed so force contributed by a receding meniscus (e.g., the first direction receding meniscus and/or the second direction receding meniscus) facilitates or improves immobilization of the capture objects. When fluid plugs are flowed, the plug's menisci generally produce flow-induced capillary forces. FIG. 2C illustrates exemplary capillary forces as arrows 153 emanating from first direction receding meniscus 151 and first direction advancing meniscus 152. One way for such a force to be generated is by flowing the fluid plug so a receding meniscus applies a capillary force with a component toward and normal to the surface comprising the assay sites. Referring to FIG. 2C again, fluid plug 130 may be flowed so first direction receding meniscus 151 applies a capillary force with component 155 normal to surface 120. In embodiments such as those involving insertion of beads into reaction vessels, such a capillary force toward the surface (and normal to a bottom of the reaction vessels) may act on the beads to push them into the wells. For example, a capillary force with component 155 normal to surface 120 may push capture object 111 into one of assay sites 110 such that capture object 111 is immobilized, as shown in FIG. 2C. Such action may in some embodiments lead to relatively efficient bead insertion. It should be understood that it is not inherent that any arbitrary receding meniscus necessarily has a capillary force with a component toward and normal to the surface, or that any such force is of sufficient magnitude to contribute to immobilization of a capture object. Instead, such a force can require provision of specific operational and dimensional parameters described in the context of the present disclosure. The inventors have determined certain suitable parameters. Operation outside such parameters may lead to a lack of a “downward” capillary force from a receding meniscus, and contrary to creating a force contributing to immobilization of a capture object as described above, may instead primarily result in capillary forces tending to move the capture objects in a direction parallel to the surface comprising the assay sites or even away from the assay sites, thereby subverting the immobilization of a capture object.

The fluid plug may be made to flow using any of a variety of techniques. For example, in some embodiments, the fluid plug acted on by a source of positive pressure (e.g., a fluid pump, a pipette or syringe) and/or a source of negative pressure (e.g., a vacuum source, a pipette or syringe). Some such embodiments may involve an apparatus (e.g., apparatus 1) configured to apply a positive and/or negative pressure differential to the fluid plug. In FIG. 3A, apparatus 1 comprises fluid pump 60—in fluid communication with fluid plug 130 on surface 120 of assay consumable 5—configured to apply such a positive and/or negative pressure differential. In some embodiments, other fluidic techniques can be employed, such as capillary force-driven flow, electrowetting-on-dielectric (EWOD) techniques, electrophoresis techniques, etc. One way an EWOD technique might be used is by configuring a fluid pump to move fluid in a channel by applying an electrical potential across two or more electrodes associated with the assay consumable. In some cases, the surface itself may be positioned so gravity-induced flow of the fluid plug can occur.

### Contact Angle

One parameter that can contribute to a receding meniscus facilitating or improving immobilization of the capture objects (e.g., via a capillary force with a component normal and toward the surface comprising the capture objects) is the contact angle of the receding meniscus during flow. The contact angle of the receding meniscus refers to the angle between the surface comprising the assay sites and the receding contact line as the fluid plug flows. As an illustration, FIG. 2F shows contact angle θ between surface 120 and receding contact line 156 of receding meniscus 151 as fluid plug 130 flows in first direction 150, in accordance with certain embodiments. It has been determined in the context of the present disclosure that having a relatively small contact angle of the receding meniscus may contribute to capillary forces from a receding meniscus facilitating capture object immobilization. The contact angle of a receding meniscus of the fluid plug can be determined, for example, by using a goniometer or equivalent piece of imaging equipment, as would be evident to one of ordinary skill in the art. The contact angle during flow may be affected and modulated using a variety of parameters, including flow pattern (e.g., substantially continuous vs. otherwise), flow rate, fluid plug composition (e.g., liquid type), immiscible fluid composition (e.g., gas type), and surface composition. For example, the strength of intermolecular interactions between the fluid plug composition and the surface composition may be selected (e.g., based on the polarity of the fluid plug and/or the hydrophobicity/hydrophilicity of the surface) so a desired contact angle is achieved. In some embodiments, the surface comprising the assay sites is or comprises a hydrophobic material (e.g., a hydrophobic polymeric material), examples of which are described in more detail below in the context of assay consumables. Certain combinations of parameters (e.g., flow rate, surface tension, viscosity) may be represented as dimensionless quantities, such as capillary number (described in more detail below). In some, but not necessarily all embodiments, operation resulting in certain ranges of such dimensionless quantities (e.g., capillary number) can afford contact angles of the receding meniscus that result in capillary forces having directionality and magnitude that facilitates immobilization of capture objects with respect to the assay sites.

In some embodiments, during at least some of the step of flowing the fluid plug across the assay sites (e.g., in the first direction), the receding meniscus (e.g., first direction receding meniscus) has a contact angle with the surface of less than 90 degrees, less than or equal to 60 degrees, less than or equal to 45 degrees, less than or equal to 30 degrees, less than or equal to 15 degrees, or less. Such low contact angles may in certain embodiments be maintained during an entirety of the step of flowing the fluid plug (e.g., a constant contact angle while being flowed in the first direction). In certain embodiments, during an entirety of the step of flowing the fluid plug across the assay sites (e.g., in the first direction), the receding meniscus (e.g., first direction receding meniscus) has a contact angle with the surface of less than 90 degrees, less than or equal to 60 degrees, less than or equal to 45 degrees, less than or equal to 30 degrees, less than or equal to 15 degrees, or less. Flowing the fluid plug in the second, different direction may be performed such the second direction receding meniscus has a contact angle within these ranges as well. This type of flow may be achieved, for example, using continuous flow techniques. This type of flow contrasts with certain conventional flow techniques used in microfluidic systems, such as conventional segmented flow techniques, which may cause changes in receding meniscus contact angle during different parts of the flow (e.g., a first contact angle while in motion, and a second different contact angle when the fluid plug is static). In some embodiments, an apparatus described comprises one or more processors configured to modulate a fluid pump (e.g., fluid pump 60 in FIGS. 3A-3B) to flow the fluid plug so it maintains a contact angle within the above ranges. For example, the one or more processors may be programmed to actuate the pump to apply an appropriate positive and/or negative pressure to the fluid plug (e.g., in a channel) to achieve a flow rate that results in the contact angles described. The modulation of the fluid pump may take into account appropriately programmed assay consumable dimensions (e.g., channel height), fluid plug and assay consumable surface material properties (e.g. relative hydrophobicity/hydrophilicity) to achieve such result.

### Flow Pattern

As mentioned above, in some embodiments, the fluid plug comprising the capture objects is made to flow so it has a substantially continuous flow pattern. As known in the art, continuous flow refers to fully developed (e.g. steady state) flow (e.g., fully developed laminar flow through narrow channels with a parabolic velocity profile), where the flow is primarily actuated by a driving force of sufficient consistency and duration to permit a fully developed flow pattern to develop—e.g. external pressure sources such as pumps and vacuum sources, capillary forces, etc. For example, a source of positive pressure to the right (or a source of negative pressure to the left) of fluid plug 130 may cause fluid plug 130 to flow in first direction 150 in FIG. 2C. Fluid pump 60 of apparatus 1 may supply such a positive pressure. Substantially continuous flow of a fluid plug is performed under conditions (large enough fluid plug size, high enough flow rate, sufficiently continuous driving force) to establish fully developed laminar flow of the plug. In some such instances, the fluid plug may be made to flow substantially continuously and with a velocity profile of the fluid plug taken in a plane parallel to the direction that is substantially parabolic as characteristic of a continuous laminar flow profile. Provision of a substantially continuous flow pattern resulting in laminar and parabolic flow of a fluid plug contrasts with flow patterns of other fluidic (e.g., microfluidic) systems, such as segmented flow, where small units of fluid in a first phase flow are completely surrounded by an immiscible fluid phase and therefore do not contact a channel wall permitting the establishment of the above described fully developed flow pattern with a parabolic flow profile. In segmented flow, small droplets of a fluid are translated in a substantially quiescent state through an immiscible fluid in which they are suspended (e.g., water droplets suspended in an immiscible oil passing through a channel). Substantially continuous flow of the fluid plug also contrasts with digital microfluidics, where small droplets of fluid are translated across small fixed distances within a channel upon discrete actuation events (e.g., electrowetting-on-dielectric techniques) of insufficient duration, consistency, and/or magnitude to result in a fully developed flow pattern—as opposed to a continuous driving force (e.g., from a pressure source) as described above as suitable in certain embodiments disclosed. In many conventional microfluidic systems and techniques, substantially continuous flow formats with laminar, parabolic flow is disfavored and viewed as less suitable or practical compared to other techniques, such as segmented flow or digital microfluidics due to potential problems such as Taylor dispersion, solute surface interactions, cross-contamination, and the need for substantial volumes of reagents and relatively long channel lengths, (see, e.g. Solvas, X. C., & DeMello, A. (2011). Droplet microfluidics: recent developments and future applications. Chemical Communications, 47(7), 1936-1942, which is incorporated by reference herein for all purposes). However, it has been determined in the context of certain disclosed embodiments that substantially continuous flow of the fluid plug under certain conditions can be effectively employed for facilitating or improving the immobilizing of capture objects with respect to assay sites. For example, maintenance of a substantially parabolic velocity profile for the fluid plug under laminar flow can result in a substantially parabolic receding meniscus shape. Such a shape may provide capillary forces with suitable directionality for facilitating capture object immobilization.

### Channel Dimensions

In some, but not necessarily all embodiments, the surface across which the fluid plug is flowed is part of a channel. The channel may be an open channel (e.g., comprising a bottom and two side) or a closed channel. For example, referring to FIG. 2F, surface 120 may be a part of a closed channel at least partially defined by surface 120 and upper surface 122, according to certain embodiments. Fluid plug 130 flows through the channel defined by surface 120 and upper surface 122. As mentioned above, the channel may be part of an assay consumable comprising the assay sites. The dimensions of a channel through which the fluid plug flows may affect the capillary forces applied by the fluid plug to the capture objects. For example, channel height 148 defined by surface 120 and upper surface 122 may affect contact angle contact angle θ between surface 120 and receding contact line 156 of receding meniscus 151 as fluid plug 130 flows in first direction 150. For example, certain channel heights relative to fluid plug volumes may facilitate substantially continuous, laminar flow with a parabolic receding meniscus. Such a parabolic receding meniscus can have a low contact angle compared to receding meniscus shapes characteristic of other flow patterns (e.g., droplets in segmented and/or digital microfluidic flow). The contact angle in turn as described affects application of force to capture objects and their immobilization with respect to assay sites. In some embodiments, the height of the channel is relatively large compared to conventional microfluidics. In some embodiments, the channel has a height of greater than or equal to 100 micrometers, greater than or equal to 150 micrometers, greater than or equal to 200 micrometers, greater than or equal to 250 micrometers, greater than or equal to 350 micrometers, greater than or equal to 400 micrometers, greater than or equal to 450 micrometers, and/or up to 500 micrometers, up to 600 micrometers, up to 800 micrometers, up to 1 mm, or more at the assay sites

### Flow Rate

As mentioned above, the flow rate of the fluid plug across the assay sites (e.g., reaction vessels) is a potential operational parameter that can affect fluid plug behavior and capture object immobilization. In some embodiments, a flow rate is selected for flowing the fluid plug (e.g., in the first direction, second direction) so the force contributed by the receding meniscus (e.g., first direction receding meniscus and/or the second direction receding meniscus) results in a downward force on the capture objects with respect to the surface comprising the assay sites. The downward force may have a component toward and normal to the surface. A meniscus shape resulting in such a downward force can be characteristic of the substantially continuous flow patterns described above (as opposed to other flow patterns such as those characteristic of turbulent flow or digital microfluidics). The flow rate of the fluid plug may be selected to contribute to such substantially continuous flow. One way the flow rate may contribute to capture object immobilization (including in some instances relatively efficient and rapid immobilization) is due to its effect on the receding meniscus contact angle. It has been determined in the context of this disclosure that the contact angle of a receding meniscus generally decreases as the flow rate (e.g., volumetric flow rate) of the fluid plug increases. It has further been determined that flowing the fluid plug at a sufficiently high flow rate may lead to a sufficiently low enough receding meniscus contact angle for capillary forces to contribute to capture object immobilization rather than, for example merely translating the capture objects laterally or away from the assay sites.

In some embodiments, the fluid plug is made to flow (e.g., in the first direction, in the second direction) at a flow rate of greater than or equal to 1 μL/s, greater than or equal to 2 μL/s, greater than or equal to 5 μL/s, greater than or equal to 10 μL/s, greater than or equal to 15 μL/s, greater than or equal to 20 μL/s, greater than or equal to 25 μL/s, greater than or equal to 30 μL/s, greater than or equal to 40 μL/s, or greater. In some embodiments, the fluid plug is flowed (e.g., in the first direction, in the second direction) at a flow rate of less than or equal to 100 μL/s, less than or equal to 80 μL/s, less than or equal to 60 μL/s, less than or equal to 50 μL/s, less than or equal to 45 μL/s, or less. Combinations of these ranges are possible. For example, in some embodiments, the fluid plug is flowed (e.g., in the first direction, in the second direction) at a flow rate of greater than or equal to 1 μL/s and less than or equal to 100 μL/s, greater than or equal to 20 μL/s and less than or equal to 100 μL/s, or greater than or equal to 40 μL/s and less than or equal to 50 μL/s. These flow rates run counter to certain conventional fluid plug flow practices in the microfluidic field, which advocate lower flow rates. One reason certain conventional microfluidic fluid plug/droplet flow techniques typically use lower flow rates (e.g., less than or equal to 10 μL/s) is because it is believed the droplets are more stable at such flow rates. It has been reported that plugs are unstable at higher fluid plug rates, e.g. in Guan, Y., Li, B., Zhu, M., Cheng, S., & Tu, J. (2019). Deformation, speed, and stability of droplet motion in closed electrowetting-based digital microfluidics. Physics of Fluids, 31(6), 062002, which is incorporated by reference herein for all purposes. But surprisingly in view of the literature, it has been determined in the context of the present disclosure that such high flow rates can under selected conditions, improve the speed and efficiency of capture object immobilization. In some embodiments, an apparatus described comprises one or more processors configured to modulate a fluid pump (e.g., fluid pump 60 in FIGS. 3A-3B) to flow the fluid plug within the above indicated unconventionally high flow rate ranges. For example, the one or more processors may be programmed to actuate the pump to apply an appropriate positive and/or negative pressure to the fluid plug (e.g., in a channel) to achieve such flow rates (e.g., so that the force contributed by a meniscus of the fluid plug—e.g., a first meniscus or a second meniscus—results in a downward force on the capture objects with respect to the surface of the assay consumable).

### Volume of Plug

In some embodiments, the fluid plug is relatively large. While typical conventional microfluidic fluid flow techniques employ relatively small droplets, e.g. for delivering suspended objects, it has been surprisingly determined in the context of the present disclosure that fluid plugs with larger volumes may be more stable and able to achieve desired flow patterns described herein relative to fluid plugs with smaller volumes. As one example, flowing relatively small fluid plugs (e.g., less than or equal to 3 μL) at relatively high flow rates (e.g., 40 μL/s) may cause unstable flow in certain environments such as relatively small channels (e.g., channels having a largest cross-sectional dimension perpendicular to a direction of flow of less than or equal to 2 mm, less than or equal to 1 mm, less than or equal to 500 microns, or less). Such unstable flow may manifest, for example, in large fluctuations in contact angle. In contrast, flowing relatively large fluid plugs (e.g., relative to channel dimensions) at similarly high flow rates may surprisingly result in stable flow, more suitable for capture object immobilization. The volume of the fluid plug, in combination with other factors such as the flow rate and nature of the driving force for the flow can contribute to achieving the flow patterns discussed here, such as substantially continuous, parabolic flow. The flow pattern may then contribute to factors such as the receding meniscus shape and contact angle. In certain instances use of fluid plugs with relatively large volumes can allow for flow at sufficiently high flow rates to achieve receding meniscus contact angles in the ranges describe above while maintaining satisfactory stability. In some embodiments, the fluid plug containing the capture objects (e.g., beads) has a volume of greater than or equal to 3 μL, greater than or equal to 10 μL, greater than or equal to 15 μL, greater than or equal to 20 μL, greater than or equal to 25 μL, greater than or equal to 30 μL, or greater. In some embodiments, the fluid plug containing the capture objects (e.g., beads) has a volume of less than or equal to 100 μL, less than or equal to 80 μL, less than or equal to 60 μL, less than or equal to 40 μL, or less than or equal to 35 μL. Combinations of these ranges are possible. For example, in some embodiments, the fluid plug containing the capture objects (e.g., beads) has a volume of greater than or equal to greater than or equal to 3μL and less than or equal to 100 μL, or greater than or equal to 20 μL and less than or equal to 50 μL.

### Capillary Number

As mentioned above, certain combinations of parameters described herein (e.g., flow rate, channel dimensions, fluid plug/immiscible fluid composition) may facilitate immobilization of capture objects with respect to the assay sites. Some such combinations of parameters may be expressed as dimensionless quantities. As one non-limiting example, in some embodiments, the fluid plug is made to flow under conditions resulting in a Capillary Number within certain ranges. Capillary Number (Cₐ) is a dimensionless quantity representing to a ratio of viscous forces to surface tension forces (at a fluid-fluid interface) during flow of a fluid, and is expressed as:

$${C}_{a} = \frac{μ V}{σ}$$

where μ is the dynamic viscosity of the fluid, V is the velocity of the fluid, and σ is the surface tension of at the interface between the fluid and is the interfacial surface tension between the fluid and an immiscible phase (e.g., a gas such as air). The Capillary Number during flow may be correlated with the contact angle of the fluid and the immiscible phase during flow. Therefore, operation of the system so that the fluid plug is made to flow within certain Capillary Number regimes may result in certain desirable contacts angles, including those resulting in capillary forces at the receding meniscus with force components pointing downward and toward the assay sites and facilitating capture object immobilization. Selecting a suitable flow rate, fluid plug composition (e.g., solvent choice), and/or channel configuration (e.g., channel height, channel cross-sectional area) can achieve Capillary Numbers facilitating a significant component of meniscus force towards the wells and relatively efficient capture object immobilization in some instances. In some embodiments, the fluid plug is made to flow under conditions resulting in a Capillary Number of greater than or equal to 1×10⁻⁶, greater than or equal to 2×10⁻⁶, greater than or equal to 5×10⁻⁶, greater than or equal to 1×10⁻⁵, greater than or equal to 2×10⁻⁵, greater than or equal to 5×10⁻⁵, greater than or equal to 1×10⁻⁴, greater than or equal to 2×10⁻⁴, greater than or equal to 5×10⁻⁴, and/or up to 1×10⁻³, up to 2 ×10⁻³, up to 5×10⁻³, or up to 1×10⁻² at 25° C. Combinations of these ranges (e.g., greater than or equal to 1×10⁻⁶ and less than or equal to 1×10⁻², greater than or equal to 1×10⁻⁴ and less than or equal to 1×10⁻³) are possible. It should be understood that other considerations related to or independent of the variables expressed in dimensionless parameters such as Capillary Number may influence immobilization of capture objects, and operation within the ranges described may not be strictly necessary in certain embodiments.

### Fluid Plug Capture Object Concentration

Each fluid plug may have a relatively low number of capture objects suspended therein per unit of volume of the fluid plug (e.g., prior to flowing the fluid plug across at least some of the assay sites). Some such “dilute” fluid plugs may be useful in delivering relatively small numbers of capture objects in proximity to the capture sites while still using relatively large fluid plugs (e.g., for greater stability of flow) as described above. This also contrasts with conventional microfluidic loading techniques that typically employ relatively large numbers of beads (e.g., greater than 200,000) in drops for delivery of the beads to assay sites. In some embodiments, the number of capture objects in the fluid plug is less than or equal to 50,000 capture objects, less than or equal to 10,000 capture objects, less than or equal to 5,000 capture objects, less than or equal to 1,000 capture objects, less than or equal to 500 capture objects, less than or equal to 200 capture objects, and/or as few as 150, as few as 100, as few as 50, as few as 10, as few as 5, as few as 1, or fewer per μL.

### Immobilization of the Capture Objects

As mentioned above, in some embodiments, at least some of the capture objects subjected to the steps of flowing the fluid plug in the first direction and/or flowing the fluid plug in the second direction become immobilized with respect to the assay sites. In certain such embodiments, the assay sites comprise reaction vessels and the capture objects are beads, at least some of which are immobilized by being inserted into the reaction vessels. FIG. 2C shows one such embodiment, where flow of fluid plug 130 in first direction 150 results in at least some of capture objects 100 in the form of beads being inserted into assay sites 110 in the form of reaction vessels. Similarly, capture objects 100 in FIGS. 3A-3B may be beads (e.g., magnetic beads), and apparatus 1 may configured to insert beads 100 into assay sites 110 in the form of reaction vessels in surface 120 of assay consumable 5. As mentioned above, force created by a receding meniscus may contribute to efficient and rapid immobilization of the capture objects, which may facilitate the use of relatively low numbers of capture objects in capture object-based assays. In some embodiments, the assay sites are at a plurality of separate locations on the surface (e.g., as an array), and the step of immobilizing at least some of the capture objects is performed so at least some of the capture objects are segregated across the plurality of separate locations. Some such embodiments may be useful in performing certain types of digital ELISA techniques.

### Synergy Between Field Strength Modulation and Fluid Flow

It has been determined in the context of this disclosure that while (1) generation of a force field (e.g., a magnetic field) in proximity to assay sites and (2) flowing a receding meniscus of a fluid plug containing the capture objects over assay sites may each alone contribute to efficient capture object immobilization, combinations of (1) and (2) can exhibit unexpected synergy and improved performance. Without wishing to be bound by any particular theory, it is believed that the generated force field may rapidly localize the capture objects in proximity to the assay sites (e.g., near openings in reaction vessels). The downward forces contributed by the receding meniscus may then encounter capture objects relatively close to the assay sites, such that the forces generated by the receding meniscus and field efficiently immobilize the capture objects. In some cases where magnetic fields are used (e.g., with a permanent magnet present under the assay sites), magnetic beads may form chains. In some such instances, the receding meniscus may encounter and break up the chains, thereby spreading the magnetic beads to facilitate bead insertion (in the case of reaction vessels). Moreover, the combined magnitudes of the force vector field and the forces applied by the receding meniscus may increase the tendency for the capture objects to move toward the assay sites.

In some embodiments, the force field (e.g., magnetic field) is present during at least a portion of step of flowing the fluid plug (e.g., in a first direction, in a second direction). However, in some embodiments, the magnitude of the force field is decreased or terminated before the step of flowing the fluid plug (e.g., in the first direction). As one example, while FIGS. 2C-2E show a force field represented by vector field 45 present while fluid plug 130 flows in first direction 150 or second direction 160, some embodiments comprise removing or reducing the force field before flowing the fluid plug in first direction 150 and/or second direction 160. Such a modulation of the force field may be useful in some instances where it is undesirable for force-field induced phenomena such as capture object-chaining to occur during flow of the receding meniscus across the assay sites. As one example, a magnetic field (e.g., from a permanent magnet and/or electromagnet) may pull magnetic beads toward a surface comprising reaction vessels, which may cause some amount of magnetic chaining. The magnetic field magnitude may be reduced or removed entirely, thereby releasing the chaining. Finally, once the magnetic beads are unchained, the receding meniscus of the fluid plug may be made to flow past at least some (or all) of the assay sites to force at least some of the unchained magnetic beads into the reaction vessels. The magnitude of the magnetic field may be reduced in the case of a permanent magnet by, force example, causing relative motion between the permanent magnet and the surface. For example, referring to FIG. 2E, when force generator 40 is a permanent magnet, the magnitude of magnetic vector field 45 can be decreased (e.g., to zero) by moving force field generator 40 in direction 146 and increasing distance 147 between force field generator 40 and the bottom of assay sites 110.

In some instances, an apparatus described can be configured to modulate a magnitude of a force field, for example by causing relative motion between the force field generator (e.g., a permanent magnet) and an assay consumable comprising a surface comprising assay sites. FIG. 3A, for example shows force field generator 40 in a first position below assay consumable 5 comprising surface 120, and FIG. 3B shows force field generator 40 in a second position at a greater distances from assay consumable 5. Such an increase in distance between the force field generator and the assay sites may reduce the magnitude of or essentially eliminate the force field at the assay sites. Alternatively or in addition to linear relative motion to increase the distance between the force field generator and the assay sites of the consumable, lateral and/or rotational motion may also be used. For example, the force field generator may be rotated within a plane such that at a first radial position the force field generator is positioned in proximity to the assay sites of the assay consumable, and at a second radial position the force field generator is positioned away from the assay sites of the assay consumable. Re-positioning (or removal) of the force field generator may be accomplished manually or using, for example, an automated translation stage of the apparatus. FIGS. 3A-3B show automated translation stage 41, for example, which can be controlled by controller 30, in some embodiments. The magnitude of the magnetic field may be modulated (e.g., decreased) with an electromagnet by, for example, adjusting a magnitude of electrical current passing through the electromagnet.

In some embodiments, at a later point in a method (e.g., after capture object immobilization), the magnitude of the force field may be increased. For example, a magnet previously removed may be reintroduced following bead insertion so the immobilized beads are kept in place during a follow up step such as a sealing step.

### Percentage of Capture Objects Immobilized

In some embodiments, a relatively large percentage of the delivered capture objects are immobilized (e.g., during the flowing steps such as in the first direction and/or second direction). While certain existing techniques for immobilizing capture objects (e.g., for capture-object based assays such as digital ELISAs) employ large excesses of capture objects relative to the number of assay sites (e.g., by a factor of 5, factor of 6, or more), certain embodiments herein use a contrary approach. It has been determined in the context of this disclosure that in some instances, delivering a relatively small number of capture objects and immobilizing a high percentage of them can allow for an assay with high sensitivity (due to the low total number of beads as described below) while generating sufficient signal from the capture objects for adequate detection. In some embodiments, at least 20%, at least 25%, at least 40%, at least 50%, at least 60%, at least 75%, at least 90%, at least 95%, at least 99%, or all of the capture objects delivered in proximity to the assay sites are immobilized during the flowing step.

### Percentage of Capture Sites Occupied

In some embodiments, a relatively small percentage of the assay sites on the surface immobilize capture objects. This approach stands in contrast to conventional approaches that, for example, strive to fill as many wells in an array with beads as possible (e.g., up to 100% of the wells filled with beads). It has been determined in the context of this disclosure that it can be advantageous instead to ensure as many capture objects are immobilized as possible rather than occupying as many assay sites as possible. One way to do so is to have a significant excess of assay sites with respect to the number of capture objects, which can result in capture objects being immobilized with respect to only a relatively small percentage of the assay sites. In some embodiments, capture objects are immobilized with respect to less than or equal to 20%, less than or equal to 15%, less than or equal to 10%, less than or equal to 5%, less than or equal to 2%, and/or as low as 1%, as low as 0.5%, as low as 0.1%, as low as 0.01% of the assay sites, or less. As an illustrative example, in FIGS. 2A-2G, surface 120 may comprise 200,000 assay sites 110 in the form of reaction vessels, and the method may cause the insertion of only 2,000 capture objects 100 in the form of beads, meaning 1% of the assay sites 110 had capture objects 100 immobilized with respect to them.

### Alternative Loading Methods

While certain embodiments described above are related to immobilizing capture objects with respect to assay sites via flow of a fluid plug simultaneous with or sequentially with generation of a force field, other formats are possible. These other formats may also provide for relatively high rapid and/or efficient capture object immobilization. For example, in some embodiments, a laterally-moving force field is applied to the delivered capture objects in proximity to the assay sites. Such a lateral force may promote movement of the capture objects across the lateral space around the assay sites on the surface, increasing the rate of capture objects interacting with assay sites. One such embodiment comprises applying a lateral force to the capture objects by adjusting a lateral distribution of the force field, and immobilizing at least some of the capture objects with respect to the assay sites via the applied lateral force. FIG. 2B shows an illustration of one such optional embodiment, where relative lateral motion, as shown by arrow 149a and arrow 149b, is created between force field generator 40, in the form of a permanent magnet, and surface 120. Such motion of force field generator 40 may move the magnetic field represented by magnetic vector field 45 laterally, which can act on capture objects 100 when they are magnetic (e.g., magnetic beads), according to some embodiments. This may cause lateral motion of the beads 100 with respect to surface 120 so they encounter and become inserted into assay sites 110. Adjusting a lateral distribution of the force field can occur while fluid 130 (e.g., a fluid plug) is stationary, or while it is in flow.

Some embodiments may comprise flowing multiple fluid plugs across at least some of the assay sites. For example, in some embodiments, a first fluid plug containing capture objects is flowed past the assay sites, followed by a second fluid plug separated from first fluid plug by an immiscible fluid, which second plug flows across at least some of the assay sites so at least at least some capture objects are immobilized with respect to the assay sites. FIG. 2G illustrates one such embodiment, where first fluid plug 130 containing capture objects 100 flows in first direction 150 across assay sites 110, and second fluid plug 230 containing capture objects 100 also flows in first direction 150 after first fluid plug 130. First fluid plug 130 and second fluid plug 230 may be separated by immiscible fluid 134 (e.g., a gas such as air). Such a method of sequential plug flow across the assay sites may, in some instances, allow for using fluid plugs with fewer capture objects (e.g., beads), and may be performed with or without the presence of a force field (e.g., magnetic field) acting on the capture objects.

### Apparatus for Immobilizing Capture Objects

As mentioned above, apparatuses for immobilization of capture objects with respect to assay sites are also described. Such apparatuses may be configured to perform certain of the methods for immobilization described above (e.g., relating to combinations of force field generation and fluid plug flow). In some embodiments, apparatuses configured to perform methods for immobilization of capture objects may further be configured to perform assays for detecting and/or quantifying analyte molecules or particles in fluid samples (such as assay described below). For example, an apparatus for immobilization of capture objects with respect to assay sites may comprise one or more components for preparing capture objects for detection (e.g., a sample washer, one or more components for incubation, etc.) or for detection or analysis (e.g., an imaging system, a computer-implemented control system). While in some embodiments such combinations of components may be integrated in a robotic system, in some embodiments, some or all of these components for sample preparation, capture objection immobilization (e.g., with respect to assay sites), and image acquisition/analysis are integrated as a microfluidic system on, for example, a single chip.

### Assay Consumable Handler

The apparatuses may in some instances comprise assay consumable handlers configured to be operatively coupled to assay consumables with surfaces comprising the assay sites. FIGS. 3A-3B depict a schematic diagram of one such assay consumable handler 10. The assay consumable handler may support and facilitate manipulation and/or positioning of the assay consumable by or within the apparatus.

The assay consumable handler may be stationary or may be movable, or at least parts thereof may be movable. For example, the assay consumable handler may be operatively associated with or comprise a stage, wherein the stage is movable. The stage may be associated with a controller configured to automatically move the stage, and/or the assay consumable handler. An assay consumable handler may be sized and/or shaped to mate with the assay consumable in certain embodiments. For example, an assay consumable handler may comprise a depressed area wherein the assay consumable may be situated and secured. Alternatively, the assay consumable handler may comprise a substantially planar surface that the assay consumable is placed upon. In some embodiments, the assay consumable handler includes fasteners (e.g., snaps, clips, clamps, ring clamps, etc.) which aid in attaching the assay consumable to the assay consumable handler, so there is little or no movement between the consumable and the consumable handler during at least certain periods of operation of the system. As another example, the assay consumable handler may utilize a vacuum or pneumatic system for securing the assay consumable. In certain embodiments, the assay consumable handler can comprise recognition elements complimentary to recognition elements of an assay consumable to facilitate proper positioning and/or to prevent use of improperly configured or counterfeit assay consumables. For example, an assay consumable may comprise a plurality of notches and the assay consumable handler may comprise a plurality of complimentary indentations. As another example, the assay consumable may comprise an RFID chip or bar code reader and the assay consumable may be required to comprise an authorized RFID chip or bar code to permit coupling of the assay consumable and the assay consumable handler without triggering an alarm condition or causing the controller to shut down operation of the system.

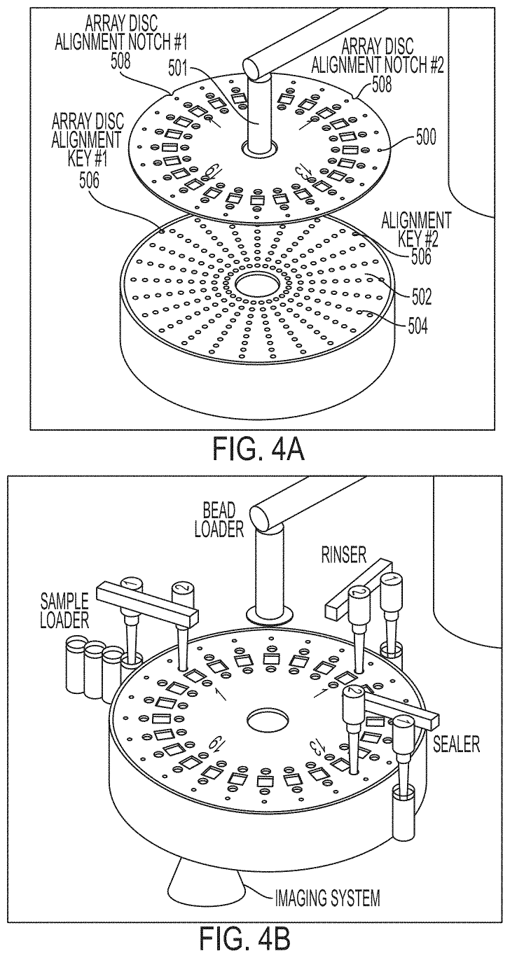

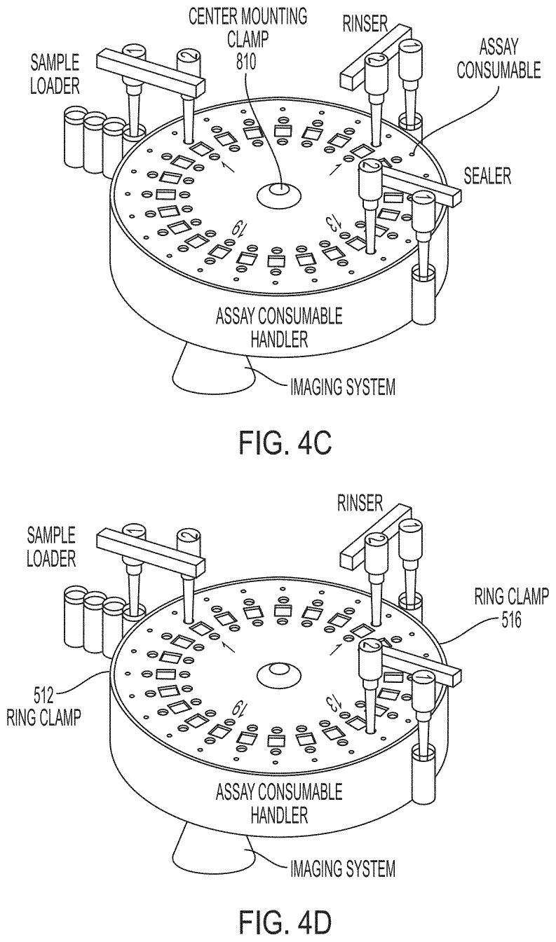

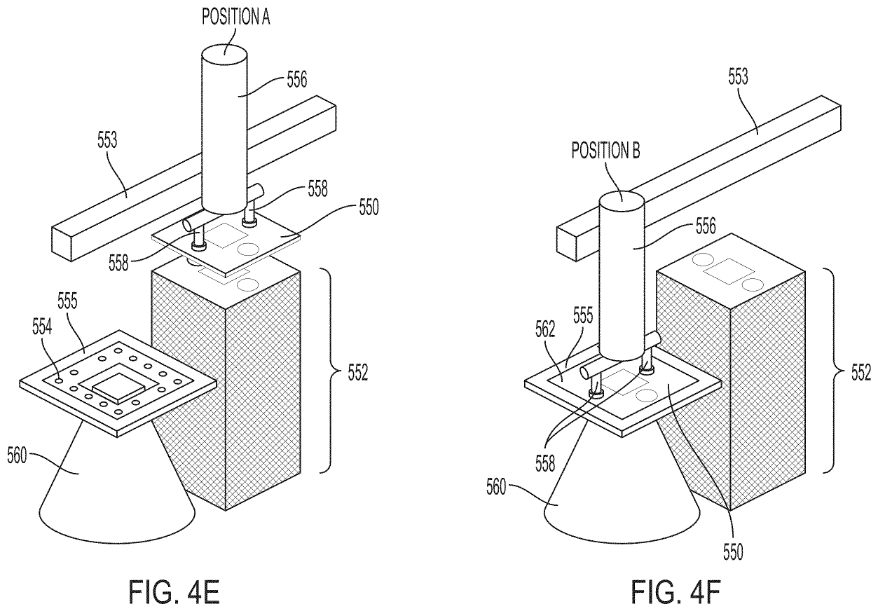

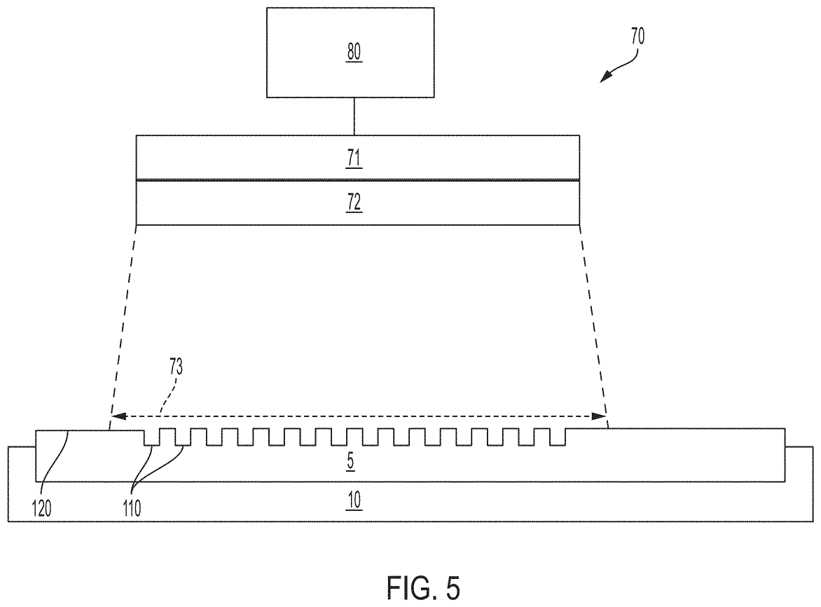

Non-limiting examples of assay consumable handlers are depicted in FIGS. 4A-4F. FIG. 4A shows assay consumable 500 and assay consumable handler 502. The apparatus comprises a component capable of moving assay consumable 500 from a first position not associated with the assay consumable handler to a position associated with assay consumable handler (e.g., arm 501). Assay consumable 500, in this example, comprises at least one notch or recognition element (e.g., notches 508) which interact specifically with a key or recognition element (e.g., keys 506) on assay consumable handler 502. Assay consumable handler 502 also comprises a plurality of holes 504 which through which a vacuum may be applied to the assay consumable. Once the assay consumable is lowered into position (e.g., as shown in FIG. 5B), where notches 508 are aligned with keys 506, vacuum may be applied to holes 504, which causes assay consumable 500 to lie flat in a secured position on the assay consumable handler. Following loading of the assay consumable on to the assay consumable handler, the handler may be positioned so the components of the apparatus (e.g., sample loader, bead loader, sealer, wiper, imaging system, etc.), are in the appropriate places. The vacuum may be maintained until the desired number of the individual groups of assay sites have been analyzed. FIG. 4C shows an assay consumable associated with the assay consumable handler via center mounting clamp 510. Center mount clamp 510 secures and holds the assay consumable flat. FIG. 4D shows an assay consumable associated with an assay consumable handler via first ring clamp 512 and second ring clamp 516. The ring clamps are configured and positioned to hold the assay consumable to the assay consumable handler by clamping the outer edges of the assay consumable.

FIGS. 4E and 4F show another example of an assay consumable handler comprising handler grabbing arm 556, cross arm 553 operatively connected with a portion of the apparatus (not shown), assay consumable handler stage 555, and assay consumable attachments 558. Also shown in the figure is imaging system 560. In FIG. 5E, single assay consumable 550 is configured to be moved from stack 552 to assay consumable stage 555. Arm 556 is in position A so arm 556 is positioned above stack 552. Assay consumable attachments 558 (e.g., suction cups, clips, etc.) are lowered to grab assay consumable 550. Handler arm 556 is moved from position A in FIG. 4E to position B in FIG. 4F along cross arm 553 so assay consumable 550 is positioned above assay consumable stage 555. FIG. 4F shows the assay consumable lowered to connect assay consumable 550 to assay consumable stage 555. In this figure, assay consumable stage 555 comprises holes 554 in fluid communication with a source of vacuum, so a vacuum may be applied to the underside of assay consumable 550 to hold it in position, as described herein (e.g., for similar, also see FIG. 4A (holes 504)). In some cases, the assay consumable handler may comprise a conveyor belt type assembly.

### Capture Object Applicator

In some embodiments, the apparatus comprises a capture object applicator. The capture object applicator may function alone in conjunction with a fluid injector and/or a fluid pump to deliver apply capture objects to the surface of an assay consumable. FIGS. 3A-3B schematically illustrate capture object applicator 20 of apparatus 1. While FIGS. 1 and 3A-3B represent capture object applicator 20 as a separate component with fluid injector 50 and/or fluid pump 60, in some embodiments these components are the same (e.g., the fluid injector may inject a fluid plug containing capture objects on to surface 120 of assay consumable 5 via a positive pressure supplied by fluid pump 60). In another example, the capture object applicator comprises a pipettor used to deliver capture objects (e.g., beads) to an entry port of a channel (e.g., microfluidic channel) to dispense it over the assay consumable. Other non-limiting examples of capture object applicators include an automated pipette associated with a fluid pump (e.g., a syringe pump, a piston-action pump, membrane pump, etc.) and microfluidic injectors. As with other components, the capture object applicator may be associated with a controller configured to automatically operate the capture object applicator.

In some embodiments, the capture object applicator is configured to apply a relatively small number of capture objects to the surface of the assay consumable or in proximity to the surface. For example, the capture object applicator may be associated with a fluid injector and/or a fluid pump adapted to produce relatively small volumes of fluid containing capture objects (e.g., beads), or to produce relatively dilute fluid plugs containing capture objects. In some embodiments, the capture object applicator is configured to apply less than or equal to 100,000, less than or equal to 50,000, less than or equal to 25,000, less than or equal to 10,000, less than or equal to 5,000, less than or equal to 2,000, less than or equal to 1,000, less than or equal to 500, less than or equal to 200, less than or equal to 100, or even as few as 50, as few as 20, as few as 10, as few as 5 capture objects, or a single capture object to the surface of the assay consumable or in proximity to the surface.

Imaging System and Detection with Fixed Field of View

Also disclosed are apparatuses and methods for imaging and/or analyzing assay sites (e.g., in the form of arrays on the surface of an assay consuming). It has been determined in the context of this disclosure that certain existing techniques for imaging assay sites do not analyze an entirety of the areas containing the assay sites, but rather a subset. By analyzing only a subset of assay sites (e.g., in determining the presence or absence of capture objects and/or associated analytes), a smaller absolute number of immobilized capture objects are analyzed than are actually immobilized. Such a loss of capture objects may be negligible in existing assays that use relatively large numbers of capture objects (e.g., greater than 100,000, greater than 200,000 or greater). However, for the presently disclosed assays that may use relatively few capture objects (e.g., less than or equal to 50,000, less than or equal to 10,000, less than or equal to 5,000, or less), such a loss of capture objects may have a significant effect on the detection of adequate signal from the capture objects. Some apparatuses described are configured to reduce or limit such a loss by analyzing an entirety of the area containing the assay sites (e.g., an array of assay sites).

In some embodiments, an apparatus for imaging an array of assay sites is provided, and may be part of an overall system for detecting and/or quantifying analytes. For example, apparatus 1 in FIG. 1 may comprise imaging system 70 and computer-implemented control system 80, in accordance with some embodiments. Imaging system 70 may be configured to capture an image of an array of assay sites on assay consumable 5, which may be oriented with respect to imaging system 70 via assay consumable handler 10. However, it should be understood that the presence of a separate assay consumable handler is optional, and some embodiments may involve directly interfacing an imaging system and an assay consumable without handling of the assay consumable by an assay consumable handler. One such embodiment may involve an apparatus for imagining an array on a microfluidic chip that can be manually operatively coupled with the imaging system. In some embodiments, the apparatus may be configured such that after immobilization of the capture objects with respect to the assay sites on the surface of the assay consumable, the imaging system can capture an image of the array without inversion of the assay consumable. For example, after immobilization of the capture objects (e.g., insertion of beads), an assay consumable handler may manipulate the assay consumable (e.g., via rotational or translational relative motion) so it is aligned with the field of view of the imaging system without inverting the assay consumable (e.g., flipping the assay consumable).

The imaging system may comprise a detector and optics. Any of a variety of detector types and optics configurations are possible, and exemplary configurations are described in more detail below. The imaging system comprising the detector and optics may have a fixed field of view greater than an area containing the array of assay sites. In some such instances, the apparatus may be configured so the array of assay sites on the assay consumable can be positioned completely within the imaging system's fixed field of view. FIG. 5 shows a schematic illustration of one such embodiment. In FIG. 5, imaging system 70 comprises detector 71 and optics 72 and is positioned over assay consumable 5 operatively coupled with assay consumable handler 10. Imaging system 70 has fixed field of view 73, which is greater than an area containing the array of assay sites 110 on surface 120 of assay consumable 5. A fixed field of view between the imaging system and the array of sites in this context refers to the imaging system capturing an image of the array of assay sites for later analysis without substantial relative motion between the field of view and the array (notwithstanding minor negligible motion). Such a fixed field of view imaging system may capture an image of the array as a “single shot”, rather than scanning across the array and generating an image as a composite of multiple images captured at a number of different relative orientations of the detector/optics and the array.

In some embodiments, the apparatus comprises a computer-implemented control system configured to receive information from the imaging system. In some such embodiments, the computer-implemented control system is configured to analyze an entirety of the area containing the array of assay sites. Referring again to FIG. 5, computer-implemented control system 80 may be configured to receive information from imaging system 70. The information may relate to an image of the array of assay sites 110 on surface 120 of assay consumable 5 (e.g., during a detection step of an assay for detecting and/or quantifying analytes in a sample). In some embodiments, computer-implemented control system 80 is configured to analyze an entirety of the area containing the array of assay sites 110. Such a configuration may allow for detection of a greater number of capture objects immobilized with respect to the assay sites than certain existing technologies that analyze only a subset of captured images. The computer-implemented control system may further be configured to determine a measure of an unknown concentration of analyte molecules or particles in an assay sample based on the analyzed entirety of the array of assay sites. In some embodiments, the computer-implemented control system is configured to analyze a relatively large area. For example, in some embodiments, the computer-implemented control system is configured to analyze an area of at least 2 mm², at least 5 mm², at least 10 mm², and/or up to 15 mm², up to 20 mm², or greater. For example, in some embodiments, the computer-implemented control system is configured to analyze at least 100,000 assay sites, at least 200,000 assay sites, at least 500,000 assay sites, or at least 1,000,000 assay sites or greater.

A variety of imaging systems potentially useful for practice of certain embodiments herein are known in the art and commercially available. Such systems and components may be adapted based upon the needs and requirements of a selected assay method being performed by the system and the technique used for detecting the analyte molecules and/or particles. For example, in some assays, the analyte molecules and/or particles are not directly detectable and additional reagents (e.g., detectable labels) are used aid in the detection. In such instances, components of the imaging system would be selected to detect such reagents.

In certain embodiments, the imaging system is configured to optically interrogate the assay sites. The sites exhibiting changes in their optical signature may be identified by a conventional optical train and optical detection system. Depending on the species to be detected and the operative wavelengths, optical filters designed for a particular wavelength may be employed for optical interrogation of the locations, as understood by those of ordinary skill in the art.

In some embodiments where optical interrogation is used, the imaging system may comprise more than one light source and/or a plurality of filters to adjust the wavelength and/or intensity of the light source. Examples of light sources include lasers, continuous spectrum lamps (e.g., mercury vapor, halogen, tungsten lamps), and light-emitting diodes (LED). For example, in some cases, a first interrogation of the assay sites may be conducted using light of a first range of wavelengths, whereas a second interrogation is conducted using light of a second, differing range of wavelengths, so the plurality of detectable molecules fluoresce.

In some embodiments, the optical signal from a plurality of assay sites is captured using a charge coupled device (CCD) camera. Other non-limiting examples of devices that can be used to capture images include charge injection devices (CIDs), complementary metal oxide semiconductors (CMOSs) devices, scientific CMOS (sCMOS) devices, time delay integration (TDI) devices, photomultiplier tubes (PMT), and avalanche photodiodes (APD). Camera variety of such devices are available from several commercial vendors.

In one embodiment, the assay consumable comprises a fiber optic bundle, and a plurality assay sites in the form of reaction vessels is formed in an end of the fiber optic bundle. According to one embodiment, the array of assay sites for the present invention can be used in conjunction with an optical detection system such as the system described in U.S. Publication No. 2003/0027126, which is incorporated by reference herein for all purposes.

Those of ordinary skill in the art will be aware that various components of the imaging system can be adapted and/or configured to provide a good image. For example, in some cases, the assay consumable is imaged through a sealing component, and thus, the imaging system can be adapted and/or configured to account for the presence of the sealing component in the optical path. As known to those of ordinary skill in the art, certain thickness of material may lead to spherical aberration and loss of resolution of the arrays. Therefore, if the sealing component is of a thickness where such aberrations occur, the optical portion of the imaging system may be designed to correct for this increased thickness. Designing the optics so fluid that matches the index of the seal material may be placed between the objective and the assay consumable can ensure that differences in the material between the objective and the seal do not lead to blurring.

Another example of a feature of the imaging system which may be configured and/or adapted to improve performance is the speed and quality of the focus capability of the imaging system. In some cases, focusing may involve using a laser focusing system based on reflection off the assay consumable surface. Laser focusing systems are commercially available. In other cases, the surface of the assay consumable comprising assay sites (which may be similar in size as the wavelength of light being processed) may include structures/fiducials built in to the assay consumable that may be used to focus the image via diffraction, refraction, absorption, reflection, fluorescence, or a combination of these and other optical phenomena.

As described above, certain embodiments of the systems and apparatus include one or more controllers and/or computer implemented control systems for operating various components/subsystems of the system, performing data/image analysis, etc. (e.g., controller 30/computer implemented control system 80 shown in FIG. 1. Any calculation methods, steps, simulations, algorithms, systems, and system elements described may be implemented and/or controlled using one or more computer implemented control system(s), such as the embodiments of computer implemented systems described below. The methods, steps, control systems, and control system elements described are not limited in their implementation to any specific computer system described, as many other different machines may be used.

The computer implemented control system(s) can be part of or coupled in operative association with an image analysis system and/or other automated system components, and, in some embodiments, is configured and/or programmed to control and adjust operational parameters, as well as analyze and calculate values, for example analyte molecule or particle concentrations as described above. In some embodiments, the computer implemented control system(s) can send and receive reference signals to set and/or control operating parameters of system apparatus. In other embodiments, the computer implemented system(s) can be separate from and/or remotely located with respect to the other system components and may be configured to receive data from one or more remote assay systems of the invention via indirect and/or portable means, such as via portable electronic data storage devices, such as magnetic disks, or via communication over a computer network, such as the Internet or a local intranet.

The computer implemented control system(s) may include several known components and circuitry, including a processing unit (i.e., one or more processors), a memory system, input and output devices and interfaces (e.g., an interconnection mechanism), as well as other components, such as transport circuitry (e.g., one or more busses), a video and audio data input/output (I/O) subsystem, special-purpose hardware, as well as other components and circuitry, as described below in more detail. Further, the computer system(s) may be a multi-processor computer system or may include multiple computers connected over a computer network.

The computer implemented control system(s) may include one or more processors, for example, a commercially available processor such as one of the series x86, Celeron and Pentium processors, available from Intel, similar devices from AMD and Cyrix, the 680X0 series microprocessors available from Motorola, and the PowerPC microprocessor from IBM. Many other processors are available, and the computer system is not limited to a particular processor.

A processor typically executes a program called an operating system, of which WindowsNT, Windows95 or 98, Windows XP, Windows Vista, Windows 7, Windows 10, UNIX, Linux, DOS, VMS, and MacOS are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer implemented control system is not limited to a particular computer platform.

The computer implemented control system(s) may include a memory system, which typically includes a computer readable and writeable non-volatile recording medium, of which a magnetic disk, optical disk, a flash memory and tape are examples. Such a recording medium may be removable, for example, a floppy disk, read/write CD or memory stick, or may be permanent, for example, a hard drive.

Such a recording medium stores signals, typically in binary form (i.e., a form interpreted as a sequence of one and zeros). A disk (e.g., magnetic or optical) has several tracks, on which such signals may be stored, typically in binary form, i.e., a form interpreted as a sequence of ones and zeros. Such signals may define a software program, e.g., an application program, to be executed by the microprocessor, or information to be processed by the application program.

The memory system of the computer implemented control system(s) also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer implemented control system(s) that implements the methods, steps, systems control and system elements control described above is not limited thereto. The computer implemented control system(s) is not limited to a particular memory system.

At least part of such a memory system described above may store one or more data structures (e.g., look-up tables) or equations such as calibration curve equations. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer implemented control system(s) may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

The computer implemented control system(s) may include one or more output devices. Example output devices include a cathode ray tube (CRT) display, liquid crystal displays (LCD), light-emitting diode (LED) displays, and other video output devices, printers, communication devices such as a modem or network interface, storage devices such as disk or tape, and audio output devices such as a speaker.

The computer implemented control system(s) also may include one or more input devices. Example input devices include a keyboard, keypad, track ball, mouse, pen and tablet, communication devices such as described above, and data input devices such as audio and video capture devices and sensors. The computer implemented control system(s) is not limited to the particular input or output devices described.

It should be appreciated that one or more of any type of computer implemented control system may be used to implement various embodiments described. Aspects of the invention may be implemented in software, hardware or firmware, or any combination thereof. The computer implemented control system(s) may include specially programmed, special purpose hardware, for example, an application-specific integrated circuit (ASIC). Such special-purpose hardware may be configured to implement one or more methods, steps, simulations, algorithms, systems control, and system elements control described above as part of the computer implemented control system(s) described above or as an independent component.

The computer implemented control system(s) and components thereof may be programmable using any of a variety of one or more suitable computer programming languages. Such languages may include procedural programming languages, for example, LabView, C, Pascal, Fortran and BASIC, object-oriented languages, for example, C++, Java and Eiffel and other languages, such as a scripting language or even assembly language.

The methods, steps, simulations, algorithms, systems control, and system elements control may be implemented using any of a variety of suitable programming languages, including procedural programming languages, object-oriented programming languages, other languages and combinations thereof, which may be executed by such a computer system. Such methods, steps, simulations, algorithms, systems control, and system elements control can be implemented as separate modules of a computer program, or can be implemented individually as separate computer programs. Such modules and programs can be executed on separate computers.

Such methods, steps, simulations, algorithms, systems control, and system elements control, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system control, or system element control, such a computer program product may comprise computer-readable signals tangibly embodied on the computer-readable medium that define instructions, for example, as part of one or more programs, that, as a result of being executed by a computer, instruct the computer to perform the method, step, simulation, algorithm, system control, or system element control.

Assays, Including Assays with Low Numbers of Beads and Efficient Loading

Methods (e.g., assays) for determining a measure of the concentration of analyte molecules or particles in a fluid sample are now described. As mentioned above, it has been unexpectedly determined in the context of this disclosure that high sensitivities for analyte detection (e.g., low limits of detection) can be achieved by using fewer capture objects in the assay compared to typical conventional approaches (e.g., certain existing digital ELISA techniques). Counterintuitively, it has been determined that gains in sensitivity achieved using fewer capture objects relative to the number of analyte molecules or particles due to an increased enzyme per bead (AEB) can outweigh potential losses in sensitivity (e.g., due increases in background signal such as from Poisson noise or less efficient analyte capture). Certain methods and systems for preparing samples and capture objects, distributing (e.g., loading/spatially segregating) capture objects, and/or detecting/analyzing capture objects described may contribute alone or cumulatively to an ability to use such low numbers of beads.

One exemplary assay format/protocol comprises exposing capture objects (e.g., beads) configured to capture a particular type of analyte molecule or particle to a solution (e.g., the fluid sample) containing or suspected of containing such analyte molecules (or particles). At least some of the analyte molecules become immobilized with respect to a capture object. The capture objects may each have affinity for a particular type of analyte molecule or particle. The capture objects may each include a binding surface having affinity for at least one type of analyte molecule (e.g., a particular type of analyte molecule or particle). In some cases, the binding surface may comprise a plurality of capture components. A “capture component”, as used herein, is any molecule, other chemical/biological entity, or solid support modification disposed upon a solid support that can specifically attach, bind or otherwise capture a target molecule or particle (e.g., an analyte molecule), so the target molecule/particle becomes immobilized with respect to the capture object. The immobilization may be caused by the association of an analyte molecule with a capture component on the surface of the capture object. In the context of immobilizing an analyte molecule or particle with respect to a capture object, “immobilized” means captured, attached, bound, or affixed so as to prevent dissociation or loss of the target molecule/particle, but does not require absolute immobility with respect to either the capture component or the object.

The number of analyte molecules immobilized with respect to a capture object may depend on the ratio of the total number of analyte molecules in the sample compared to at least one of the total number, size, and/or surface density of capture components of capture objects provided. In some embodiments, the number of molecules or particles immobilized with respect to a single capture object may follow a standard Poisson distribution. In some cases, a statistically significant number of the capture objects associate with a single analyte molecule or particle from the fluid sample and a statistically significant number of capture objects do not associate with any analyte molecule or particle from the fluid sample. In some embodiments, the percentage of capture objects which associate with at least one analyte molecule (e.g., of the particular type of analyte molecule or particle) is less than or equal to 99.999%, less than or equal to 99.99%, less than or equal to 99.9%, less than or equal to 99%, less than or equal to 98%, less than or equal to 95%, less than or equal to 90%, less than or equal to 80%, less than or equal to 70%, less than or equal to 60%, less than or equal to 50%, less than or equal to 40%, less than or equal to 30%, less than or equal to 20%, less than or equal to 10%, less than or equal to 5%, less than or equal to 1%, less than or equal to 0.5%, less than or equal to 0.1%, or less of the total number of capture objects.

### Low Number of Capture Objects Exposed to the Sample Solution

In some embodiments, the number of capture objects exposed to the solution containing or suspected of containing the analyte molecules or particles is relatively low. As mentioned above, the unconventional use of relatively low numbers of capture objects (e.g., during exposure to the analyte molecules or particles and/or during downstream analysis and detection steps) may, in some instances, impart unexpected and otherwise unappreciated increases in sensitivity (e.g., level of detection). Certain teachings of this disclosure related to efficient handling of capture objects may help overcome known challenges related to handling and detecting such small numbers of capture objects that have dissuaded others from using such small numbers of capture objects (e.g., in an ultrasensitive digital ELISA assay). In some embodiments, the number of capture objects (e.g., having affinity for a particular type of analyte molecule or particle) exposed to the solution containing or suspected of containing the analyte molecules or particles is less than or equal to 50,000, less than or equal to 7,500, less than or equal to 5,000, less than or equal to 4,000, less than or equal to 3,000, less than or equal to 2,000 or fewer. In some embodiments, the number of capture objects (e.g., having affinity for a particular type of analyte molecule or particle) exposed to the solution containing or suspected of containing the analyte molecules or particles is greater than or equal to 100, greater than or equal to 200, greater than or equal to 500, greater than or equal to 1,000, or more. In some embodiments, the number of capture objects (e.g., having affinity for a particular type of analyte molecule or particle) exposed to the solution containing or suspected of containing the analyte molecules or particles is less than or equal 10,000. Combinations of these ranges are possible. For example, in some embodiments, the number of capture objects (e.g., having affinity for a particular type of analyte molecule or particle) exposed to the solution containing or suspected of containing analyte molecules or particles is greater than or equal to 100 and less than or equal to 50,000, greater than or equal to 100 and less than or equal to 10,000, or greater than or equal to 100 and less than or equal to 5,000.

In some embodiments, compositions having relatively small numbers of capture objects, with relatively low concentrations of analyte may be used. Such compositions may be produced during any of several method steps described, or may be provided separately. It has been unexpectedly determined in the context of this disclosure that compositions having a relatively few capture objects may be used in assays for detecting low concentrations of analyte. The preparation of such compositions runs counter to conventional wisdom, which typically advocates use of a large number of capture objects (to increase chances of analyte capture, or to avoid challenges of handling/detection). In some embodiments, the composition is an isolated fluid having a volume of between 10 and 1000 microliters, between 50 and 500 microliters, or between 100 and 350 microliters. Some such compositions have at least one type of analyte molecule or particle present at a concentration of between 0.001 attomolar (aM) and 10 picomolar (pM), between 0.01 aM and 1 pM, between 0.1 aM and 100 femtomolar (fM), or between 1 and 10 fM. In some embodiments, the composition comprises between 100 and 10,000 or between 1,000 and 5,000 capture objects (e.g., beads) including a binding surface having affinity for the at least one type of analyte molecule or particle.

### Incubation Duration

It has been determined in the context of this disclosure that the duration of exposure of the capture objects to the solution containing or suspected of containing the analyte molecules or particles can influence the extent to which analyte molecules are immobilized with respect to capture objects. Exposing the capture objects to the solution (e.g., in an incubation step) for a relatively long period of time may cause a higher percentage of the analyte molecules or particles in the solution to be immobilized with respect to the capture objects; surprisingly even in instances where relatively few capture objects are present (e.g., less than or equal to 10,000, less than or equal to 5,000, or fewer). It is believed that a relatively long exposure (e.g., incubation) may overcome kinetic limitations afforded by the presence of fewer capture objects (e.g., in some instances when immobilization is controlled by bimolecular reaction kinetics). In some embodiments, the capture objects are exposed to the solution containing or suspected of containing at least of type of analyte molecule or particle (e.g., a fluid sample) for greater than or equal to 15 minutes, greater than or equal to 30 minutes, greater than or equal to 1 hour, greater than or equal to 2 hours, greater than or equal to 4 hours, greater than or equal to 6 hours, greater than or equal to 8 hours, greater than or equal to 10 hours, greater than or equal to 12 hours, and/or up to 18 hours, up to 24 hours, up to 30 hours, or longer.

### Sample Volume

It has been determined in the context of this disclosure that the volume of the solution (e.g., fluid sample) to which the capture objects are exposed can influence the extent to which analyte molecules become immobilized with respect to the capture objects. Exposing the capture objects to a relatively large volume of solution (e.g., in an incubation step) may provide one way to use relatively dilute solutions (e.g., from dilute samples) with relatively few capture objects (e.g., less than or equal to 50,000, less than or equal to 10,000, less than or equal to 5,000, or fewer). It is believed that in some instances larger volumes of solution during the exposing step can result in relatively high assay sensitivities (e.g., compared to otherwise equivalent assays using smaller volumes) by providing a larger number of analyte molecules or particles that can immobilize with respect to the capture objects. A larger number of analytes may then increase the ratio of detectable species per capture object during the assay (e.g., average enzymes per bead) and, potentially, the assay sensitivity. In some embodiments, the solution containing or suspected of containing at least of type of analyte molecule or particle (e.g., a fluid sample) has a volume of greater than or equal to 50 microliters, greater than or equal to 100 microliters, greater than or equal to 200 microliters, greater than or equal to 300 microliters, and/or up to 400 microliters, up to 500 microliters, up to 1 mL, or greater.

### Spatially Segregating and Addressing Capture Objects

In some embodiments, an assay method employs a step of spatially segregating capture objects into a plurality of separate locations to facilitate detection/quantification. In some such embodiments, the segregation is performed so each location comprises/contains either zero or one or more analyte molecule or particle from the fluid sample. Additionally, in some embodiments, the locations may be configured in a manner so each location can be individually addressed. In some embodiments, a measure of the concentration of an analyte molecule or particle in a fluid sample may be determined by detecting analyte molecules or particles immobilized with respect to a binding surface having affinity for at least one type of analyte molecule or particle (e.g., a particular type of molecule or particle). In certain embodiments the binding surface may form (e.g., a surface of an assay site such as a well/reaction vessel on a substrate) or be contained within (e.g., a surface of a capture object, such as a bead, immobilized with respect to an assay site such as a well) one of a plurality of locations (e.g., assay sites such as wells/reaction vessels) on a substrate (e.g., plate, dish, chip, optical fiber end, surface of a channel, disc, surface of an assay consumable, etc.). At least a portion of the locations may be addressed and a measure indicative of the number or fraction of capture objects associated with at least one analyte molecule or particle from the fluid sample may be made. In some cases, based at least in part upon the measure indicative of the number or fraction, a measure of the concentration of analyte molecules or particles in the fluid sample may be determined. In some cases, a measure of the concentration may be based at least in part on the number or fraction of locations determined to contain a capture object that is or was associated with at least one analyte molecules or particle. The measure of the concentration of analyte molecules or particles in the fluid sample may be determined by a digital analysis method/system optionally employing Poisson distribution adjustment and/or based at least in part on a measured intensity of a signal, as known to those of ordinary skill in the art. For example, in some embodiments in which a measure indicative of a number or fraction of capture objects determined to be associated with an analyte molecule or particle represents a relatively low percentage (e.g., less than or equal to 80%, less than or equal to 70%, less than or equal to 50%, or less), a digital analysis method (optionally employing a Poisson distribution adjustment) may be used, at least in part, to determine a measure of the concentration of the analyte molecule or particle in the fluid sample.

However, in some embodiments in which a measure indicative of a number or fraction of capture objects determined to be associated with an analyte molecule or particle is determined to represent a relatively higher percentage (e.g., greater than or equal 50%, greater than or equal 60%, greater than or equal 70%, greater than or equal to 80%, greater than or equal to 90%), the measure indicative of a concentration of the analyte molecule or particle in the fluid sample can be determined, at least in part, based on a measurement of an intensity level of at least one signal (e.g., fluorescence signal) indicative of the presence of an analyte molecule or particle. In some embodiments, the method comprises, based upon the measure indicative of the number or fraction of capture objects associated with at least one analyte molecule or particle from the fluid sample, either determining a measure of the concentration of analyte molecules or particles in the fluid sample based at least in part on the measure indicative of the number or fraction of capture objects determined to be associated at least one analyte molecule or particle, or determining a measure of the concentration of analyte molecules or particles in the fluid sample based at least in part on a measured intensity level of a signal that is indicative of the presence of a plurality of analyte molecules or particles. In certain embodiments, an automated system configured and programmed to perform the assay and determine the measure indicative of a concentration of the analyte molecule or particle in the fluid sample may be programmed to initially determine a measure indicative of the fraction of capture objects determined associated with an analyte molecule or particle—e.g. the fraction of assay sites displaying a positive signaling status and/or an average intensity level of the capture sites—and to automatically (or manually in response to a prompt provided to a user) switch which measurement and quantification technique is employed (i.e. a digital analysis method—optionally employing a Poisson distribution adjustment, or an analog intensity level based method). The use of such digital and/or “analog” methods for determining a measure indicative of a concentration of an analyte molecule or particle, alone or in combination, is described, for example, in U.S. patent application Ser. No. 13/037,987, filed Mar. 1, 2011, published as US-2011-0245097 on Oct. 6, 2011, entitled “METHODS AND SYSTEMS FOR EXTENDING DYNAMIC RANGE IN ASSAYS FOR THE DETECTION OF MOLECULES OR PARTICLES,” by Rissin et al., which is incorporated by reference herein in its entirety for all purposes. In some cases, the assay methods and/or systems may be automated.

It should be understood that while in some instances a measure indicative of the number or fraction of capture objects associated with at least one analyte or molecule may be determined at least in part by addressing the separate locations (e.g., assay sites), other techniques of determining the measure indicative of the number or fraction are possible. For example, in some embodiments at least some of the capture objects subjected to the exposing and immobilizing steps are individually addressed (e.g., by being individually isolated from a remainder of the capture objects). One non-limiting way of individually addressing capture objects without necessarily spatially segregating the capture objects into a plurality of separate locations is by flowing at least some of the capture objects through a channel (e.g., a microchannel having a largest cross-sectional dimension with respect to the direction of flow of less than or equal to 1 mm, less than or equal to 500 micrometers, or less) and addressing the flowed capture objects. For example, the capture objects may flow past a detector (e.g., an optical detector) and be addressed accordingly.

In some embodiments, the capture objects (e.g., some of which may be associated with at least one analyte molecule or particle and optionally) may be provided as separate droplets or as objects contained within droplets (e.g., by being segregated using fluidic techniques such as microfluidic techniques). In some such embodiments, the capture objects comprise or are each contained within a liquid droplet suspended in a fluid immiscible with the liquid droplets. The liquid droplets may be suspended in a fluid immiscible with the liquid droplets at least during a step of individually addressing the capture objects (e.g., via a detector). In some instances the liquid droplets may be provided as an array (e.g., by being spatially segregated such as on a substantially planar surface). However, in some instances the liquid droplets may be individually addressed by being flowed through a channel (e.g., a microchannel) and interrogated while flowing through the channel. One way the droplets may be interrogated is by flowing the droplets past a detector. For example, the detector may be an optical detector. In some such embodiments the droplets are temporally segregated with respect to a fixed detection location, for example by being flowed through a channel (e.g., during an addressing step) past such a detection location. While the droplets may be flowed single file in some instances, single file flow is not necessary in all cases. For example, the droplets may be collected in a layer and all droplets imaged substantially simultaneously.

### Spatially Segregating a High Percentage of Capture Objects

In some embodiments, a relatively high percentage of capture objects are spatially segregated into the plurality of separate locations (e.g., assay sites such as reaction vessels). Such an approach is contrary to the prevailing practices in the field of ultrasensitive detection where typically a relatively small percentage (e.g., less than 20%) of the total number of capture objects (e.g., having affinity for a particular type of molecule or particle) exposed to the analyte molecules or particles are segregated into separate locations (e.g., by being immobilized with respect to assay sites) and a large excess of capture objects are discarded. The prevailing approach therefore focuses on immobilizing capture objects with respect to a high percentage of the locations at the expense of using a large excess of capture objects. By instead spatially segregating a high percentage of capture objects into the separate locations, the use of a relatively small total number of capture objects in the assay may be possible, thereby increasing sensitivity in some instances. In some embodiments, at least 25%, at least 30%, at least 50%, at least 60%, at least 75%, at least 90%, at least 95%, at least 99%, or all of the capture objects (e.g., having affinity for a particular type of molecule or particle) subjected to the exposing and immobilizing steps described above are spatially segregated into the plurality of separate locations (e.g., assay sites such as reaction vessels).

Certain methods and systems which employ spatially segregating analyte molecules or particles are known in the art and may be employed (with appropriate modifications according to the present disclosure), and are described in U.S. Patent Application Publication No. US-2007-0259448 (Ser. No. 11/707,385), filed Feb. 16, 2007, entitled “METHODS AND ARRAYS FOR TARGET ANALYTE DETECTION AND DETERMINATION OF TARGET ANALYTE CONCENTRATION IN SOLUTION,” by Walt et al.; U.S. Patent Application Publication No. US-2007-0259385 (Ser. No. 11/707,383), filed Feb. 16, 2007, entitled “METHODS AND ARRAYS FOR DETECTING CELLS AND CELLULAR COMPONENTS IN SMALL DEFINED VOLUMES,” by Walt et al.; U.S. Patent Application Publication No. US-2007-0259381 (Ser. No. 11/707,384), filed Feb. 16, 2007, entitled “METHODS AND ARRAYS FOR TARGET ANALYTE DETECTION AND DETERMINATION OF REACTION COMPONENTS THAT AFFECT A REACTION,” by Walt et al.; International Patent Publication No. WO 2009/029073 (International Patent Application No. PCT/US2007/019184), filed Aug. 30, 2007, entitled “METHODS OF DETERMINING THE CONCENTRATION OF AN ANALYTE IN SOLUTION,” by Walt et al.; U.S. Patent Application Publication No. US-2010-0075862 (Ser. No. 12/236,484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al.; U.S. Patent Application Publication No. US-2010-00754072 (Ser. No. 12/236,486), filed Sep. 23, 2008, entitled “ULTRA-SENSITIVE DETECTION OF MOLECULES ON SINGLE MOLECULE ARRAYS,” by Duffy et al.; U.S. Patent Application Publication No. US-2010-0075439 (Ser. No. 12/236,488), filed Sep. 23, 2008, entitled “ULTRA-SENSITIVE DETECTION OF MOLECULES BY CAPTURE-AND-RELEASE USING REDUCING AGENTS FOLLOWED BY QUANTIFICATION,” by Duffy et al.; International Patent Publication No. WO2010/039179 (International Patent Application No. PCT/US2009/005248), filed Sep. 22, 2009, entitled “ULTRA-SENSITIVE DETECTION OF MOLECULES OR ENZYMES,” by Duffy et al.; U.S. Patent Application Publication No. US-2010-0075355 (Ser. No. 12/236,490), filed Sep.23, 2008, entitled “ULTRA-SENSITIVE DETECTION OF ENZYMES BY CAPTURE-AND-RELEASE FOLLOWED BY QUANTIFICATION,” by Duffy et al.; U.S. patent application Ser. No. 12/731,130, filed Mar. 24, 2010, published as US-2011-0212848 on Sep. 1, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026657, filed Mar. 1, 2011, published as WO 2011/109372 on Sep. 9, 2011, entitled “ULTRA-SENSITIVE DETECTION OF MOLECULES USING DUAL DETECTION METHODS,” by Duffy et al.; U.S. patent application Ser. No. 12/731,135, filed Mar. 24, 2010, published as US-2011-0212462 on Sep. 1, 2011, entitled “ULTRA-SENSITIVE DETECTION OF MOLECULES USING DUAL DETECTION METHODS,” by Duffy et al.; International Patent Application No. PCT/US2011/026665, filed Mar. 1, 2011, published as WO 2011/109379 on Sep. 9, 2011, entitled “METHODS AND SYSTEMS FOR EXTENDING DYNAMIC RANGE IN ASSAYS FOR THE DETECTION OF MOLECULES OR PARTICLES,” by Rissin et al.; U.S. patent application Ser. No. 12/731,136, filed Mar. 24, 2010, published as US-2011-0212537 on Sep. 1, 2011, entitled “METHODS AND SYSTEMS FOR EXTENDING DYNAMIC RANGE IN ASSAYS FOR THE DETECTION OF MOLECULES OR PARTICLES,” by Duffy et al.; U.S. patent application Ser. No. 13/035,472, filed Feb. 25, 2011, published as US 2012-0196774, entitled “SYSTEMS, DEVICES, AND METHODS FOR ULTRA-SENSITIVE DETECTION OF MOLECULES OR PARTICLES,” by Fournier et al.; U.S. patent application Ser. No. 13/037,987, filed Mar. 1, 2011, published as US-2011-0245097 on Oct. 6, 2011, entitled “METHODS AND SYSTEMS FOR EXTENDING DYNAMIC RANGE IN ASSAYS FOR THE DETECTION OF MOLECULES OR PARTICLES,” by Rissin et al.; each of which are incorporated by reference in their entirety for all purposes.

In some embodiments, a measure indicative of the number or fraction of locations containing a capture object but not associated with an analyte molecule or particle analyte molecule or particle is also determined and/or a measure indicative of the number or fraction of locations not containing any capture object is also determined. In some such embodiments, a measure of the concentration of analyte molecules or particles in the fluid sample may be based at least in part on the ratio of the number of locations determined to contain a capture object associated with an analyte molecule or particle to the total number of locations determined to contain a capture object not associated with an analyte molecule or particle, and/or a measure of the concentration of analyte molecule or particle in the fluid sample may be based at least in part on the ratio of the number of locations determined to contain a capture object associated with an analyte molecule or particle to the number of locations determined to not contain any capture objects, and/or a measure of the concentration of analyte molecule or particle in the fluid sample may be based at least in part on the ratio of the number of locations determined to contain a capture object associated with an analyte molecule or particle to the number of locations determined to contain a capture object. In yet other embodiments, a measure of the concentration of analyte molecules or particles in a fluid sample may be based at least in part on the ratio of the number of locations determined to contain a capture object and an analyte molecule or particle to the total number of locations addressed and/or analyzed.

In certain embodiments, at least some of the capture objects (e.g., at least some associated with at least one analyte molecule or particle from the fluid sample) are spatially separated into a plurality of locations, for example, assays sites such as reaction vessels in an array format. The reaction vessels may be formed in, on and/or of any suitable material, and in some cases, the reaction vessels can be sealed or may be formed upon the mating of a substrate with a sealing component, as discussed in more detail below. In certain embodiments, especially where quantization of the capture objects associated with at least one analyte molecule or particle is desired, the partitioning of the capture objects can be performed so at least some (e.g., a statistically significant fraction; e.g., as described in International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS,” by Duffy et al., incorporated by reference herein for all purposes) of the reaction vessels comprise at least one or, in certain cases, only one capture object associated with at least one analyte molecule or particle and at least some (e.g., a statistically significant fraction) of the reaction vessels comprise a capture object not associated with any analyte molecules or particles. The capture objects associated with at least one analyte molecule or particle may be quantified in certain embodiments, thereby allowing for the detection and/or quantification of analyte molecules or particles in the fluid sample by techniques described in more detail herein.

An exemplary assay method may proceed as follows. A solution containing or suspected of containing analyte molecules or particles is provided. The solution may be a fluid sample (e.g., a biological fluid or derived from a biological fluid). An assay consumable comprising assay sites (e.g., in an array) is exposed to the solution. In some cases, the analyte molecules or particle are provided in a manner (e.g., at a concentration) so at least some (e.g., a statistically significant fraction) of the assay sites contain a single analyte molecule or particle and a statistically significant fraction of the assay sites do not contain any analyte molecules or particles. The assay sites may optionally be exposed to a variety of reagents (e.g., using a reagent loader) and/or rinsed. The assay sites may then optionally be sealed and imaged (using systems or methods described in this disclosure or in, for example, U.S. patent application Ser. No. 13/035,472, filed Feb. 25, 2011, published as US 2012-0196774, entitled “SYSTEMS, DEVICES, AND METHODS FOR ULTRA-SENSITIVE DETECTION OF MOLECULES OR PARTICLES,” by Fournier et al.). The images are then analyzed (e.g., using a computer implemented control system) so a measure of the concentration of the analyte molecule or particle in the fluid sample may be obtained, based at least in part, by determination of a measure of the number or fraction of assay sites which contain an analyte molecule or particle and/or the number or fraction which do not contain any analyte molecule or particles. In some cases, the analyte molecules or particles are provided in a manner (e.g., at a concentration) so at least some assay sites comprise more than one analyte molecule or particle. In such embodiments, a measure of the concentration of analyte molecule or particle in the fluid sample may be obtained at least in part on an intensity level of at least one signal indicative of the presence of a plurality of analyte molecules or particles at one or more of the assay sites.

In some cases, the methods optionally comprise exposing the fluid sample to beads (e.g., having affinity for a particular type of molecule or particle) (e.g., magnetic beads). The total number of beads (e.g., having affinity for a particular type of molecule or particle) may be relatively small (e.g., less than or equal to 50,000), as described above. At least some of the analyte molecules or particle are immobilized with respect to a bead. In some cases, the analytes molecule or particles are provided in a manner (e.g., at a concentration) such that a statistically significant fraction of the beads associate with a single analyte molecule or particle and a statistically significant fraction of the beads do not associate with any analyte molecules or particles. At least some of the beads (e.g., those associated with a single analyte molecule or particle or not associated with any analyte molecule or particle) may then be spatially separated/segregated such that they are immobilized with respect to assay sites (e.g., of an assay consumable). The assay sites (e.g., comprising reaction vessels) may optionally be exposed to a variety of reagents and/or rinsed. At least some of the assay sites may then be addressed to determine the number of assay sites containing an analyte molecule or particle. In some cases, the number of assay sites containing a bead not associated with an analyte molecule or particle, the number of assay sites not containing a bead and/or the total number of assay sites addressed may also be determined. Some such determination(s) may then be used to determine a measure of the concentration of analyte molecule or particles in the fluid sample. In some cases, more than one analyte molecule or particle may associate with a bead and/or more than one bead may be present in an assay site. In some cases, the analyte molecule or particles are exposed to at least one additional reaction component before, concurrent with, and/or following spatially separating at least some of the analyte molecule or particles such that they are immobilized with respect to the assay sites.

The analyte molecule or particles may be directly detected or indirectly detected. With direct detection, an analyte molecule or particle may comprise a molecule or moiety that may be directly interrogated and/or detected (e.g., a fluorescent entity). With indirect detection, an additional component is used for determining the presence of the analyte molecule or particle. For example, the analyte molecules or particles (e.g., optionally associated with a bead) may be exposed to at least one type of binding ligand. In certain embodiments, a binding ligand may be adapted to be directly detected (e.g., the binding ligand comprises a detectable molecule or moiety) or may be adapted to be indirectly detected (e.g., including a component that can convert a precursor labeling agent into a labeling agent). A component of a binding ligand may be adapted to be directly detected in embodiments where the component comprises a measurable property (e.g., a fluorescence emission, a color, etc.). A component of a binding ligand may facilitate indirect detection, for example, by converting a precursor labeling agent into a labeling agent (e.g., an agent detected in an assay). A “precursor labeling agent” is any molecule, particle, or the like, that can be converted to a labeling agent upon exposure to a suitable converting agent (e.g., an enzymatic component). A “labeling agent” is any molecule, particle, or the like, that facilitates detection, by acting as the detected entity, using a chosen detection technique. In some embodiments, the binding ligand may comprise an enzymatic component (e.g., horseradish peroxidase, beta-galactosidase, alkaline phosphatase, etc.). A first type of binding ligand may or may not be used in conjunction with additional binding ligands (e.g., second type, etc.).

More than one type of binding may be employed in any assay method, for example, a first type of binding ligand and a second type of binding ligand. In one example, the first type of binding ligand is able to associate with a first type of analyte molecule or particle and the second type of binding ligand is able to associate with the first binding ligand. In another example, both a first type of binding ligand and a second type of binding ligand may associate with the same or different epitopes of an analyte molecule or particle.

In some embodiments, a binding ligand and/or an analyte molecule or particle may comprise an enzymatic component. The enzymatic component may convert a precursor labeling agent (e.g., an enzymatic substrate) into a labeling agent (e.g., a detectable product). A measure of the concentration of analyte molecules or particles in the fluid sample can then be determined based at least in part by determining the number or fraction of capture objects associated with a labeling agent (e.g., by relating the number of locations containing a labeling agent to the number of locations containing a capture object). Other non-limiting examples of systems or methods for detection include embodiments where nucleic acid precursors are replicated into multiple copies or converted to a nucleic acid that can be detected readily (e.g., by introducing a detectable moiety such as a fluorescent moiety). Some such methods include the polymerase chain reaction (PCR), rolling circle amplification (RCA), ligation, Loop-Mediated Isothermal Amplification (LAMP), etc. Such systems and methods will be known to those of ordinary skill in the art, for example, as described in “DNA Amplification: Current Technologies and Applications,” Vadim Demidov et al., 2004.

In some embodiments, the binding ligand comprises a particle. For example, the binding ligand may comprise a particle having a surface having affinity for (e.g. by having a molecule immobilized to the surface having affinity for) the same particular type of analyte molecule or particle as does the capture object. In some embodiments, an analyte molecule or particle is immobilized with respect to a capture object having a surface having affinity for that particular analyte molecule or particle, and a binding ligand comprising a particle having affinity for that same analyte molecule or particle becomes immobilized with respect to the immobilized analyte molecule or particle, resulting in a complex comprising the capture object and the binding ligand each associated with the analyte molecule or particle. In some embodiments, a first binding ligand becomes immobilized with respect to an immobilized analyte molecule or particle, and a second binding ligand comprising a particle becomes immobilized with respect to the immobilized first binding ligand. In some embodiments, the particle associated with the binding ligand can be detected. Particles associated with binding ligands may be detected via any of a variety of techniques. For example, detecting the presence of a binding ligand comprising a particle (and therefore the presence of an immobilized analyte molecule or particle) can involve detecting emission of electromagnetic radiation from the particle. As one such example, the particle associated with the binding ligand may be excited via irradiation with light, and the particle may emit electromagnetic radiation via fluorescence that can be detected. Quantum dots and semiconducting polymer dots (Pdots) are examples of types of fluorescent particles that may be employed. In some embodiments, the particle emits electromagnetic radiation via photon upconversion, where two or more lower energy incident photons are absorbed by the particle (e.g. a nanometer sized nanoparticle) and converted into one emitted photon having a higher energy (shorter wavelength). Such upconverting nanoparticles are known, and include, for example, lanthanide and actinide-doped transition metal containing nanoparticles. In some embodiments, the presence of a binding ligand comprising a particle can be detected via electromagnetic radiation scattering (e.g., optical scattering), for example through use of a plasmonic particle associated with the binding ligand. As one specific such example, the plasmonic particle may be a gold nanoparticle whose light scattering can be affected by binding to other species such as an analyte molecule or particle. In some embodiments, the binding ligand may be associated with a magnetic (e.g., superparamagnetic or ferromagnetic) particle, and detection of the presence of the particle may be involve magnetic phenomenon associated with the particle (e.g., detection of a magnetic field from or affected by the magnetic particle). Any of a variety of types and/or sizes of particles may be used, depending on, for example, the detection technique to be employed. The particle may be, for example, a nanoparticle having a largest cross-sectional dimension of less than or equal to 100 nm, or the particle may be larger (e.g., a bead having a cross-sectional dimension greater than or equal to 100 nm and less than or equal to 100 micrometers).

Another exemplary embodiment of indirect detection is as follows. In some cases, the analyte molecules or particles are exposed to a precursor labeling agent (e.g., enzymatic substrate) and the enzymatic substrate is converted to a detectable product (e.g., fluorescent molecule) upon exposure to the analyte molecule or particle.

The assay methods and systems may employ a variety of components, steps, and/or other aspects known and understood by those of ordinary skill in the art. For example, a method may further comprise determining at least one background signal determination (e.g., and further comprising subtracting the background signal from other determinations), wash steps, etc. In some cases, the assays or systems may include the use of at least one binding ligand, as described herein. In some cases, the measure of the concentration of analyte molecule or particle in a fluid sample is based at least in part on comparison of a measured parameter to a calibration curve. The calibration curve may be developed using samples containing known concentrations of target analyte molecules or particles. In some instances, the calibration curve is formed at least in part by determination at least one calibration factor.

In certain embodiments, solubilized, or suspended precursor labeling agents may be employed, wherein the precursor labeling agents are converted to labeling agents which are insoluble in the liquid and/or which become immobilized within/near the location (e.g., within an assay site such as a reaction vessel in which the labeling agent is formed). Some such precursor labeling agents and labeling agents and their use is described in commonly owned U.S. Patent Application Publication No. US-2010-0075862 (Ser. No. 12/236,484), filed Sep. 23, 2008, entitled “HIGH SENSITIVITY DETERMINATION OF THE CONCENTRATION OF ANALYTE MOLECULES OR PARTICLES IN A FLUID SAMPLE,” by Duffy et al., which is incorporated by reference herein for all purposes.

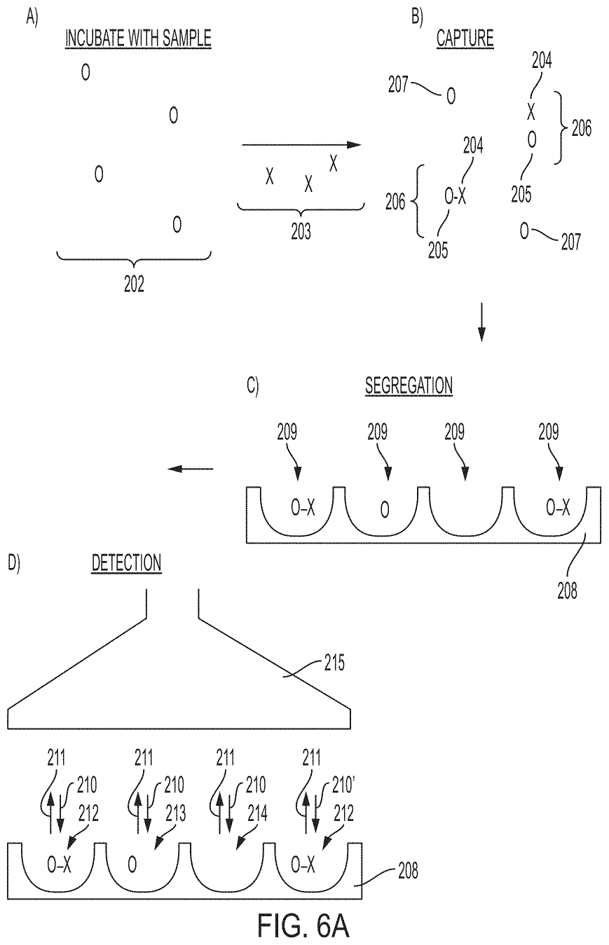

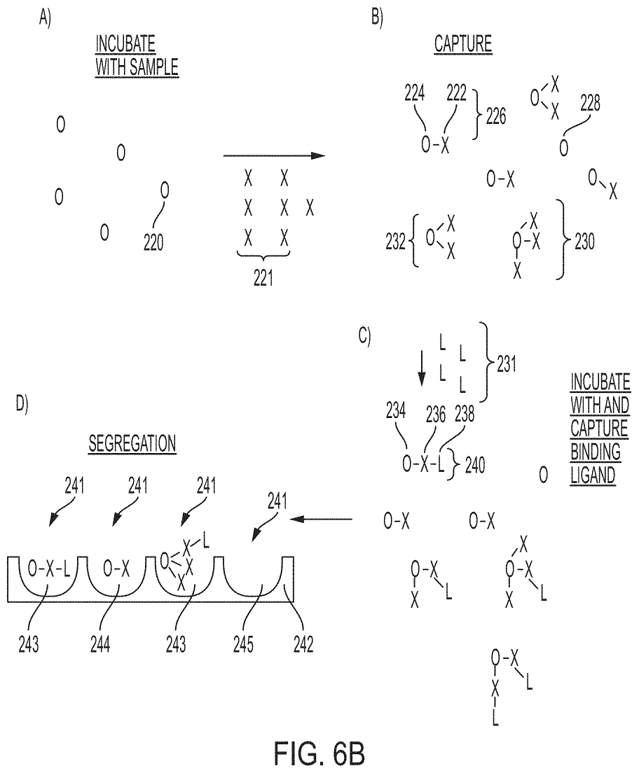

An exemplary embodiment of an assay method that may be used in certain embodiments of the invention is illustrated in FIG. 6A. Capture objects 202 are provided (step (A)). In this example, the capture objects comprises a plurality of beads. The beads are exposed to a fluid sample containing analyte molecules 203 (e.g., beads 202 are incubated with analyte molecules 203). At least some of the analyte molecules are immobilized with respect to a bead. In this example, the analyte molecules are provided in a manner (e.g., at a concentration) so a statistically significant fraction of the beads associate with a single analyte molecule and a statistically significant fraction of the beads do not associate with any analyte molecules. For example, as shown in step (B), analyte molecule 204 is immobilized with respect to bead 205, thereby forming complex 206, whereas some beads 207 are not associated with any analyte molecules. It should be understood, in some embodiments, more than one analyte molecule may associate with at least some of the beads, as described herein. At least some of the plurality of beads (e.g., those associated with a single analyte molecule or not associated with any analyte molecules) may then be spatially separated/segregated into a plurality of separate locations. As shown in step (C), the plurality of locations is illustrated as substrate 208 comprising a plurality of assay sites in the form of wells/reaction vessels 209. In this example, each reaction vessel comprises either zero or one bead. At least some of the reaction vessels may then be addressed (e.g., optically or via other detection means) to determine the number of locations containing a bead associated with an analyte molecule. For example, as shown in step (D), the plurality of reaction vessels are interrogated optically using light source 215, wherein each reaction vessel is exposed to electromagnetic radiation (represented by arrows 10) from light source 215. The light emitted (represented by arrows 211) from each reaction vessel is determined (and/or recorded) by detector 215 (in this example, housed in the same system as light source 215). A measure indicative of the number or fraction of reaction vessels containing a bead associated with an analyte molecule (e.g., reaction vessels 212) is determined based on the light detected from the reaction vessels. In some cases, a measure indicative of the number or fraction of reaction vessels containing a bead not associated with an analyte molecule (e.g., reaction vessel 213), a measure indicative of the number or fraction of wells not containing a bead (e.g., reaction vessel 214) and/or a measure indicative total number of wells addressed may also be determined. Such determination(s) may then be used to determine a measure of the concentration of analyte molecules in the fluid sample.

A non-limiting example of an embodiment where a capture object is associated with more than one analyte molecule is illustrated in FIG. 6B. Capture objects 220 are provided (step (A)). In this example, the capture objects comprise beads. The beads are exposed to a fluid sample containing analyte molecules 221 (e.g., beads 220 are incubated with analyte molecules 221). At least some of the analyte molecules are immobilized with respect to a bead. For example, as shown in step (B), analyte molecule 222 is immobilized with respect to bead 224, thereby forming complex 226. Also illustrated is complex 230 comprising a bead immobilized with respect to three analyte molecules and complex 232 comprising a bead immobilized with respect to two analyte molecules. Additionally, in some cases, some of the beads may not associate with any analyte molecules (e.g., bead 228). The beads from step (B) are exposed to binding ligands 231. As shown in step (C), a binding ligand associates with some of the analyte molecules immobilized with respect to a bead. For example, complex 240 comprises bead 234, analyte molecule 236, and binding ligand 238. The binding ligands are provided in a manner such that a statistically significant fraction of the beads comprising at least one analyte molecule become associated with at least one binding ligand (e.g., one, two, three, etc.) and a statistically significant fraction of the beads comprising at least one analyte molecule do not become associated with any binding ligands. At least some of the plurality of beads from step (C) are then spatially separated into a plurality of separate locations. As shown in step (D), in this example, the locations comprise assay sites in the form of reaction vessels 241 on substrate 242.

The plurality of reaction vessels may be exposed to the beads from step (C) so each reaction vessel contains zero or one bead. The substrate may then be analyzed to determine a measure indicative of the number or fraction of reaction vessels containing a binding ligand (e.g., reaction vessels 243), wherein the number or fraction may be related to a measure of the concentration of analyte molecules in the fluid sample. In some cases, a measure indicative of the number or fraction of reaction vessels containing a bead and not containing a binding ligand (e.g., reaction vessel 244), a measure indicative of the number or fraction number of reaction vessels not containing a bead (e.g., reaction vessel 245), and/or the total number of reaction vessels addressed/analyzed may also be determined. Some such determination(s) may then be used to determine a measure of the concentration of analyte molecules in the fluid sample.

### Multiplexed Assays

It should be understood that while in some embodiments a single type of analyte molecule or particle is detected/quantified (“singleplex”), in other embodiments, more than one type of analyte molecule or particle is detected/quantified (“multiplex”). Certain methods described relating to the use of relatively low numbers of capture objects during analyte exposure and/or spatially segregating a relatively high percentage of capture objects may be particularly advantageous in such multiplex assays. For example, conventional multiplex assays involving the detection or determination of a concentration of both a first type of analyte molecule or particle and a second type of analyte molecule or particle may involve the use of a greater number of capture objects than in singleplex assays. When relatively large numbers of capture objects are used for each of the first type of analyte molecule or particle and the second type of analyte molecule or particle, the additional capture objects involved in multiplex assays can result in very large total numbers of capture objects having affinity for any type of analyte molecule or particle, which can make loading and sealing of capture objects in assay sites difficult or impractical due to high solid masses that cannot easily be pushed off the surface using oil or can lead to high levels of capture object aggregation (e.g., in an assay device). However, with relatively low numbers of capture objects having affinity for each type of analyte molecule or particle, (e.g., less than or equal to 50,000, less than or equal to 10,000, or less) fewer total capture objects are involved, so steps such as sealing of the capture objects in assay sites can be achieved with oil and with little to no aggregation. Additionally, it is known that signals and binding events relating to different analytes or particles may complicate detection of the different analytes due to “cross-talk” (e.g., during substantially simultaneous detection in arrays of assay sites). It has been realized in the context of the present disclosure that the use of relatively low numbers of capture objects can reduce or eliminate such cross-talk (e.g., by resulting in greater distances between immobilized capture objects). Some such multiplex assays also benefit from the sensitivity improvements from using lower number of capture objects (e.g., beads) for capture of each individual analyte.

In some embodiments, different capture objects for analyte capture of different analyte targets may be employed. In some cases, different sub-groups of the total group of capture objects have different binding specificity (e.g., by including surfaces with differing binding specificity). In these embodiments, more than one type of analyte molecule may be quantified and/or detected in a single, multiplex assay method. For example, the capture objects described above may be first capture objects each having affinity for a first type of analyte molecule or particle, the method may further comprise exposing second capture objects each having an affinity for a second type of analyte molecule to the solution. Upon exposure to a sample containing the first type of analyte molecule and the second type of analyte molecule, the first type of analyte molecule becomes immobilized with respect to the first capture objects and the second type of analyte molecule becomes immobilized with respect to the second capture objects. The first capture objects and the second capture objects may be encoded to be distinguishable from each other (e.g., to facilitate differentiation upon detection) by including a differing detectable property. For example, each sub-group of capture object may have a differing fluorescence emission, a spectral reflectivity, shape, a spectral absorption, or an FTIR emission or absorption. In a particular embodiment, each sub-group of the total group of capture objects comprises one or more dye compounds (e.g., fluorescent dyes) but at varying concentration levels, such that each sub-group of capture object has a distinctive signal (e.g., based on the intensity of the fluorescent emission). In some embodiments involving spatial segregation, upon spatially segregating the capture objects after the capture step into a plurality of locations for detection, a location comprising a first capture object associated with a first type of analyte molecule can be distinguished from a location comprising a second capture object associated with a second type of analyte molecule via detection of the differing property. The number of locations comprising each sub-group of capture object and/or the number of capture objects associated with an analyte molecule may be determined, allowing a determination of a measure of the concentration of both the first type of analyte molecule and the second type of analyte molecules in the fluid sample based at least in part on these numbers. It should be understood that while some multiplexing methods may involve detection of two different types of analytes molecules or particles (e.g., a first type of analyte molecule or particle and a second type of analyte molecule), some methods further comprise detection of greater numbers of different types of analyte molecules or particles (e.g., a third type of analyte molecule or particle, a fourth type of analyte molecule or particle, and so on). A multiplex assay may involve detection of at least 1, at least 2, at least 3, at least 4, at least 5, at least 10, at least 20, at least 50, and/or up to 100, up to 120, up to 150, or more different types of analyte molecules or particles. The total number of capture objects having affinity for any type of analyte molecule or particle employed in an assay may scale with the number of different types of analyte molecules or particles to be detected. For example, a singleplex assay may involve 50,000 or fewer total capture objects (each having affinity for a particular type of analyte molecule or particle), while a “duplex” assay may involve 100,000 or fewer total capture objects (50,000 or fewer having affinity for a first type of analyte molecule or particle and 50,000 or fewer having affinity for a second type of analyte molecule or particle). In some multiplex assays, the number of capture objects in each sub-group of capture objects (each sub-group having affinity for different types of analyte molecules or particles) is less than or equal to 50,000, less than or equal to 25,000, less than or equal to 10,000, less than or equal to 5,000, less than or equal to 2,000, and/or as low as 1,000, as low as 500, as low as 200, as low as 100, or lower) during the step of exposure to the solution. In some embodiments, the total number of capture objects having affinity for any type of analyte molecule or particle is less than or equal to 100,000, less than or equal to 80,000, less than or equal to 60,000, less than or equal to 50,000, less than or equal to 25,000, less than or equal to 10,000, less than or equal to 5,000, and/or as low as 2,000, as low as 1,000, as low as 500, as low as 200, as low as 100, or lower during the step of exposure to the solution.

In some embodiments, a plurality of locations may be addressed and/or a plurality of capture objects and/or species/molecules/particles of interest may be detected substantially simultaneously. “Substantially simultaneously” when used in this context, refers to addressing/detection of the locations/capture objects/species/molecules/particles of interest at approximately the same time such that the time periods during which at least two locations/capture objects/species/molecules/particles of interest are addressed/detected overlap, as opposed to being sequentially addressed/detected, where they would not. Simultaneous addressing/detection can be accomplished by using various techniques, including optical techniques (e.g., CCD or CMOS detectors). Spatially segregating capture objects and analyte molecules or particles into a plurality of discrete, resolvable locations, according to some embodiments facilitates substantially simultaneous detection by allowing multiple locations to be addressed substantially simultaneously. For example, for embodiments where individual analyte molecules or particles are associated with capture objects spatially segregated with respect to the other capture objects into a plurality of discrete, separately resolvable locations during detection, substantially simultaneously addressing the plurality of discrete, separately resolvable locations permits individual capture objects, and thus individual analyte molecules or particles to be resolved. For example, in certain embodiments, individual analyte molecules/particles of a plurality of analyte molecules/particles are partitioned across a plurality of reaction vessels so each reaction vessel contains zero or only one species/molecule/particle. In some cases, at least 10%, at least 20%, at least 30%, at least 40%, at least 50%, at least 60%, at least 70%, at least 80%, at least 85%, at least 90%, at least 95%, at least 96%, at least 97%, at least 98%, at least 99%, at least 99.5% of all analyte molecules or particles are spatially separated with respect to other analyte molecules or particles. A plurality of analyte molecules or particles may be detected substantially simultaneously within a time period of less than or equal to 1 second, less than or equal to 500 milliseconds, less than or equal to 100 milliseconds, less than or equal to 50 milliseconds, less than or equal to 10 milliseconds less than or equal to 1 millisecond, less than or equal to 500 microseconds, less than or equal to 100 microseconds, less than or equal to 50 microseconds less than or equal to 10 microseconds, less than or equal to 1 microsecond less than or equal to 0.5 microseconds, less than or equal to 0.1 microseconds, less than or equal to 0.01 microseconds, less than or equal to 0.001 microseconds, or less. In some embodiments, the plurality of analyte molecules or particles may be detected substantially simultaneously within a time period of between about 100 microseconds and about 0.001 microseconds, between about 10 microseconds and about 0.01 microseconds, or less.

In some embodiments, the capture objects and/or the locations are optically interrogated. The capture objects and/or locations exhibiting changes in their optical signature may be identified by a conventional optical train and optical detection system. Depending on the detected species (e.g., type of fluorescence entity, etc.) and the operative wavelengths, optical filters designed for a particular wavelength may be employed for optical interrogation of the locations. In embodiments where optical interrogation is used, the system may comprise more than one light source and/or a plurality of filters to adjust the wavelength and/or intensity of the light source. In some embodiments, the optical signal from a plurality of locations is captured using a CCD or CMOS camera.

In some embodiments of the present invention, the assay sites (e.g., reaction vessels) may be sealed (e.g., after introducing the capture objects, analyte molecules or particles, binding ligands, and/or precursor labeling agent), for example, through the mating of the substrate and a sealing component. Sealing the assay sites (e.g., reaction vessels) may be such that the contents of each assay site cannot escape the assay site during the remainder of the assay. In some cases, the assay sites (e.g., reaction vessels) may be sealed after adding the capture objects, and, optionally, at least one type of precursor labeling agent to facilitate detection of the analyte molecules or particles. For embodiments employing precursor labeling agents, by sealing the contents in some or each assay site (e.g., reaction vessel), a reaction to produce the detectable labeling agents can proceed within the assay sites (e.g., reaction vessels), thereby producing a detectable amount of labeling agents retained in the assay site for detection.

In some embodiments, at least some (e.g., a subset or all) of the assay sites are not sealed (e.g., after introducing the capture objects, analyte molecules or particles, binding ligands, and/or precursor labeling agent). In some such instances, a detection signal production process of the assay does not produce freely diffusible detectable molecules (e.g., labeling agents), thereby avoiding diffusion-related interference of signal at capture objects resulting from labeling agents diffusing away from other capture objects (which could reduce accuracy of the assay). For example, in some embodiments, labeling agents are generated from precursor labeling agents and immobilized (e.g., via chemical bonds or precipitation) with respect to the capture objects and/or other surfaces at or near the capture objects, as described in more detail below. Such immobilization of labeling agents can result in spatially fixed detectable signals on or in proximity to the signal-generating capture objects that do not appreciably diffuse from the analyte-signal-generating capture objects (e.g., those associated with analyte molecules or particles) to non-analyte-signal-generating capture objects (e.g., those not associated with any analyte molecules or particles). In some embodiments, less than or equal to 50%, less than or equal to 25%, less than or equal to 10%, less than or equal to 5%, less than or equal to 2%, less than or equal to 1%, or none of the assay sites are sealed during the assay or certain steps of the assay (e.g., during an addressing step). As such, in some embodiments, an apparatus for immobilizing capture objects and/or performing an assay described herein need not include a sealer.

The plurality of locations (e.g., assay sites) may be formed may be formed using a variety of methods and/or materials. In some embodiments, the plurality of locations comprises assay sites in the form of reaction vessels/wells on a substrate. In some cases, the reaction vessels may in some instances be formed as an array of depressions on a first surface. In other cases, however, the reaction vessels may be formed by mating a sealing component comprising a plurality of depressions with a substrate that may either have a featureless surface or include depressions aligned with those on the sealing component. Any of the device components, for example, the substrate or sealing component, may be fabricated from a compliant material, e.g., an elastomeric polymer material, to aid in sealing. The surfaces may be or made to be hydrophobic or contain hydrophobic regions. Hydrophobicity may in some instances reduce leakage of aqueous samples from the reaction vessels (e.g., microwells). The reactions vessels, in certain embodiments, may be configured to receive and contain only a single capture object (e.g., bead).

In some embodiments, the assay sites (e.g., reaction vessels) may all have approximately the same volume. In other embodiments, the assay sites (e.g., reaction vessels) may have differing volumes. The volume of each individual assay site (e.g., reaction vessel) may be selected to be appropriate to facilitate any particular assay protocol. For example, in one set of embodiments where it is desirable to limit the number of capture objects used for analyte capture immobilized with respect to each site to a small number, the volume of the assay sites (e.g., reaction vessels) may range from attoliters or smaller to nanoliters or larger depending upon the nature of the capture objects, the detection technique and equipment employed, the number and density of the assay sites (e.g., reaction vessels) on the substrate and the expected concentration of capture objects in the fluid applied to the substrate containing the wells. In one embodiment, the size of the assay site (e.g., reaction vessel) may be selected such only a single capture object used for analyte molecule or particle capture can be fully contained within the assay site (e.g., reaction vessel) (see, for example, U.S. patent application Ser. No. 12/731,130, filed Mar. 24, 2010, published as US-2011-0212848 on Sep. 1, 2011, entitled “ULTRA-SENSITIVE DETECTION OF MOLECULES OR PARTICLES USING BEADS OR OTHER CAPTURE OBJECTS,” by Duffy et al.; International Patent Application No. PCT/US2011/026645, filed Mar. 1, 2011, published as WO 2011/109364 on Sep. 9, 2011, entitled “ULTRA-SENSITIVE DETECTION OF MOLECULES OR PARTICLES USING BEADS OR OTHER CAPTURE OBJECTS ,” by Duffy et al., each herein incorporated by reference for all purposes).

The total number of locations and/or density of the locations employed in an assay (e.g., the number/density of reaction vessels in an array) can depend on the composition and end use of the array. As mentioned above, the number of assay sites (e.g., reaction vessels) employed may depend on the number of types of analyte molecule or particle and/or binding ligand employed, the suspected concentration range of the assay, the method of detection, the size of the capture objects, the type of detection entity (e.g., free labeling agent in solution, precipitating labeling agent, etc.). In some embodiments, the number of capture objects exposed to the solution containing or suspected of containing at least one analyte molecule or particle is less than or equal to the number of locations employed in the assay (e.g., number of assay sites on the surface such as in an array). In some embodiments, the ratio of the number of capture objects exposed to the solution containing or suspected of containing at least one analyte molecule or particle to the number of separate locations (e.g., assay sites) employed in the assay is less than or equal to 1:1, less than or equal to 1:2, less than or equal to 1:3, less than or equal to 1:4, less than or equal to 1:5, less than or equal to 1:10, less than or equal to 1:20, less than or equal to 1:30, less than or equal to 1:40, and/or as low as 1:50, as low as 1:100, as low as 1:1,000, as low as 1:2,000, as low as 1:5,000, or less.

Arrays containing from about 2 to many billions of assay sites (e.g., reaction vessels) (or total number of reaction vessels) can be made by utilizing a variety of techniques and materials. Increasing the number of assay sites (e.g., reaction vessels optionally in the form of an array) can increase the dynamic range of an assay or to allow multiple samples or multiple types of analytes to be assayed in parallel. An array may comprise between one thousand and one million assay sites (e.g., reaction vessels) per sample to be analyzed. In some cases, the array comprises greater than one million assay sites (e.g., reaction vessels). In some embodiments, the array comprises between 1,000 and about 50,000, between 1,000 and 1,000,000, between 1,000 and 10,000, between 10,000 and 100,000, between 100,000 and 1,000,000, between 100,000 and 500,000, between 1,000 and 100,000, between 50,000 and 100,000, between 20,000 and 80,000, between 30,000 and 70,000, between 40,000 and 60,000 assay sites (e.g., reaction vessels). In some embodiments, the array comprises 10,000, 20,000, 50,000, 100,000, 150,000, 200,000, 300,000, 500,000, 1,000,000, or more, assay sites (e.g., reaction vessels). The assay sites (e.g., reaction vessels) may have a volume in any of the ranges described above (e.g., greater than or equal to 10 attoliters and less than or equal to 100 picoliters, greater than or equal to 1 femtoliter and less than or equal to 1 picoliter).

The assay sites (e.g., reaction vessels), optionally in the form of an array, may be arranged on a substantially planar surface or in a non-planar three-dimensional arrangement. The assay sites (e.g., reaction vessels) may be arrayed in a regular pattern or may be randomly distributed. In a specific embodiment, the array is a regular pattern of sites on a substantially planar surface permitting the sites to be addressed in the X-Y coordinate plane.

In some embodiments, the assay sites (e.g., reaction vessels) are formed on and/or in a solid material. The solid material may be part of, for example, an assay consumable described herein. Such a solid material may be or comprise a hydrophobic material. As will be appreciated by those in the art, the number of potentially suitable materials in which the reaction vessels can be formed is very large, and includes, but is not limited to, glass (including modified and/or functionalized glass), plastics (including acrylics, polystyrene and copolymers of styrene and other materials, polypropylene, polyethylene, polybutylene, polyurethanes, cyclic olefin copolymer (COC), cyclic olefin polymer (COP), Teflon®, polysaccharides, nylon or nitrocellulose, etc.), elastomers (such as poly(dimethyl siloxane) and poly urethanes), composite materials, ceramics, silica or silica-based materials (including silicon and modified silicon), carbon, metals, optical fiber bundles, or the like. The substrate material may be selected to allow for optical detection without appreciable autofluorescence. In certain embodiments, the assay sites (e.g., reaction vessels) may be formed in a flexible material.

A reaction vessel in a surface (e.g., substrate or sealing component) may be formed using a variety of techniques known in the art, including, but not limited to, photolithography, stamping techniques, molding techniques, etching techniques, or the like. As will be appreciated by those of the ordinary skill in the art, the technique used can depend on the composition and shape of the supporting material and the size and number of reaction vessels. In a particular embodiment, an array of reaction vessels is formed by creating microwells on one end of a fiber optic bundle and utilizing a planar compliant surface as a sealing component.

In some embodiments, the assays and methods described may be carried out on commercially available systems, for example, the Simoa HD-1 Analyzer™, Simoa HD-X Analyzer™, and Quanterix SR-X™ (Quanterix™, Lexington, Mass.). See also U.S. patent application Ser. No. 13/035,472, filed Feb. 25, 2011, published as US 2012-0196774, entitled “SYSTEMS, DEVICES, AND METHODS FOR ULTRA-SENSITIVE DETECTION OF MOLECULES OR PARTICLES,” by Fournier et al., herein incorporated by reference. In some instances modifications to the Simoa HD-1 Analyzer™ and Quanterix SR-X™ can be made to facilitate certain methods and systems described above regarding the generation of force fields and flowing of fluid plugs.

Alternatively, the equivalent structures of reaction vessels may be fabricated using other methods and materials that do not utilize the ends of an optical fiber bundle as a substrate. For example, the array may be a spotted, printed or photolithographically fabricated substrate produced by techniques known in the art; see for example WO95/25116; WO95/35505; PCT US98/09163; U.S. Pat. Nos. 5,700,637, 5,807,522, 5,445,934, 6,406,845, and 6,482,593, each of which are incorporated by reference herein for all purposes. In some cases, the array may be produced using molding, embossing, and/or etching techniques as known to those of ordinary skill in the art.

In some embodiments, the plurality of locations comprise assay sites that are not a plurality of reaction vessels/wells. For example, in embodiments where capture objects are employed, a patterned substantially planar surface may be employed and the patterned areas form a plurality of locations. In some cases, the patterned areas may comprise substantially hydrophilic surfaces which are substantially surrounded by substantially hydrophobic surfaces. In certain embodiments, a capture objects (e.g., beads) may be substantially surrounded by a substantially hydrophilic medium (e.g., comprising water), and the capture objects may be exposed to the patterned surface so the capture objects associate in the patterned areas (e.g., the hydrophilic locations on the surface), thereby spatially segregating the beads. For example, in one such embodiment, a substrate may be or include a gel or other material able to provide a sufficient barrier to mass transport (e.g., convective and/or diffusional barrier) to prevent capture objects used for analyte capture and/or precursor labeling agent and/or labeling agent from moving from one location on or in the material to another location to cause interference or cross-talk between spatial locations containing different capture objects during the time frame required to address the locations and complete the assay. For example, in one embodiment, capture objects are spatially separated by dispersing the capture objects on and/or in a hydrogel material. In some cases, a precursor labeling agent may be already present in the hydrogel, thereby facilitating development of a local concentration of the labeling agent (e.g., upon exposure to a binding ligand or analyte molecule carrying an enzymatic component). As still yet another embodiment, the capture objects may be confined in one or more capillaries. In some cases, the capture objects may be absorbed or localized on a porous or fibrous substrate, for example, filter paper. In some embodiments, the capture objects may be spatially segregated on a uniform surface (e.g., a planar surface), and the capture objects may be detected using precursor labeling agents which are converted to substantially insoluble or precipitating labeling agents that remain localized at or near the location of where the corresponding capture object is localized. In some cases, single analyte molecules or particles may be spatially segregated into a plurality of droplets. That is, single analyte molecules or particles may be substantially contained in a droplet containing a first fluid. The droplet may be substantially surrounded by a second fluid, wherein the second fluid is substantially immiscible with the first fluid.

Immobilizing Labeling Agent with Respect to Capture Object

In some embodiments, precursor labeling agents are converted to labeling agents that become immobilized with respect to the capture objects. As one example, a freely diffusible precursor agent may be exposed to a binding ligand immobilized with respect to an analyte molecule or particle that is itself immobilized with respect to a capture object (e.g., a bead). That freely diffusible precursor agent can undergo a chemical reaction facilitated by a component of the binding ligand (e.g., an enzymatic component) to form a labeling agent that, upon formation or following a further chemical or physical transformation and/or translocation (e.g., a further chemical reaction and/or deposition), becomes immobilized with respect to such capture object (e.g., bead) such that the labeling agent does not freely diffuse from the capture object. The immobilized labeling agent can produce a detectable signal (e.g., emission of electromagnetic radiation such as from fluorescence) at (e.g., on) the capture object indicative of the presence of at least one analyte molecule or particle associated with the capture object. In some such embodiments, a measure indicative of the number or fraction of capture objects having at least one immobilized labeling agent can then be determined. A measure of the concentration of a particular analyte molecule or particle can then be determined based at least in part on that measure indicative of the number or fraction of capture objects determined to have at least one immobilized labeling agent.

It has been recognized in the context of the present disclosure that immobilized labeling agents (as opposed to freely diffusible labeling agents) can allow for simplified sample handling and/or detection schemes. For example, a lack of freely diffusible labeling agents may facilitate capture object detection methods that do not involve sealing the capture objects and labeling agents in spatially and fluidically isolated assay sites (e.g., sealed reaction vessels such as sealed microwells) because, at least in part, immobilized labeling agents do not appreciably diffuse away from the capture objects with which they are associated to the interfere with signal detection from capture objects not associated with any analyte molecules or particles (which can lead to inaccurate measures indicative of the number or fraction of capture objects associated with an analyte molecule or particle and therefore inaccurate measures of the concentration of the analyte molecule or particle as described above).

In some embodiments, the process of converting precursor labeling agents into labeling agents immobilized with respect to the capture objects associated with the analyte molecules or particles occurs prior to spatial segregation of the capture objects into a plurality of separate locations (e.g., separate assay sites such as separate reaction vessels). In some embodiments, the process of converting precursor labeling agents into labeling agents immobilized with respect to the capture objects associated with the analyte molecules or particles occurs after spatial segregation of the capture objects into a plurality of separate locations (e.g., separate assay sites such as separate reaction vessels or separate locations on a planar surface).

The labeling agent produced from the precursor labeling agent may become immobilized with respect to the capture object in any of a variety of ways. For example, the capture object may have a solid surface on which the labeling agent may become immobilized upon or following formation from the precursor labeling agent. Such immobilization may occur via formation of a chemical bond between the labeling agent and a functional group attached to the capture object (e.g., a functional group attached to the surface of a bead). Such a chemical bond may be a covalent bond. In some embodiments, immobilization of the labeling agent with respect to the capture object occurs via a non-covalent interaction. One such example is an affinity-based specific binding interaction between the labeling agent and a species (e.g., a biomolecule, a functional group) attached to the surface of the capture object. In some embodiments, a detectable moiety is immobilized with respect to the labeling agent following formation of the chemical bond between the labeling agent and a species associated with the capture object. For example, an added detectable moiety may associate with the immobilized labeling agent via a covalent bond or non-covalent interaction (e.g., hybridization or a non-covalent specific affinity association) during and/or after immobilization of the labeling agent. In some embodiments, the labeling agent is immobilized via a non-specific chemical or physical interaction with a surface of the capture object. For example, in some embodiments, the labeling agent is immobilized via formation of a substantially insoluble or precipitating species that binds to or otherwise associates with the capture object. For example, the labeling agent may be substantially insoluble in a liquid in which the capture object is present or the labeling agent may be present at a local concentration above a solubility limit of the labeling agent such that the labeling agent precipitates or otherwise is deposited on the capture object (e.g., as a film or particulate precipitate on the surface of the capture object).

As a specific set of illustrative examples of some embodiments involving conversion of a precursor labeling agent into a labeling agent immobilized with respect to a capture object via an enzymatic component of a binding ligand, binding ligands having a component comprising horseradish peroxidase (HRP) will be discussed. HRP is a common enzymatic component for various assays and is known to those of ordinary skill in the art. HRP may be the enzymatic component of a binding ligand capable of associating with an analyte molecule or particle, and/or another binding ligand (which may in turn be capable of associating with the analyte molecule or particle). As a non-limiting example, wherein the analyte molecule is an antigen, a binding ligand may be an HRP-labeled antibody or streptavidin conjugate. In some cases, HRP converts a precursor labeling agent molecule into a labeling agent molecule that is substantially insoluble under the operative conditions and precipitates onto the capture object. Many examples of precursor labeling agents are known and include those typically used in Western blotting applications, such as chloronaphthol and/or diaminobenzidine. In some instances, a precipitate is a darkly colored molecule allowing the precipitate to be detected optically. For example, darkly colored precipitates may be detected using light when the precipitate absorbs light differently than does the surface of a capture object that lacks such darkly colored precipitate.

A binding ligand that comprises an enzymatic component (e.g., HRP) may be used jointly with a precursor labeling agent molecule (e.g., enzymatic substrate) that may be immobilized (e.g., via formation of a chemical bond with a functional group attached to the surface of the capture object) when converted to a labeling agent molecule (e.g., detectable product). For example, HRP in the presence of hydrogen peroxide catalyzes the conversion of tyramide into an activated tyramide (e.g., as a free radical) that can become immobilized with respect to materials of certain capture objects. For example, the capture objects may have surfaces comprising functional groups (e.g., hydroxy-containing groups such as phenol groups) that can react with free radicals of active tyramide to form covalent bonds that attach the tyramide to the surface of the capture object. Typically short lifetimes (<1 ms) of the activated tyramide can prevent significant diffusion of the activated tyramide away from the site of its formation (e.g., in some instances the labeling radius is limited to 20 nm). In such manner, most or all tyramide molecules will tend to immobilize locally with respect to capture objects associated with the binding ligands having the horseradish peroxidase components. In some embodiments, a precursor labeling agent such as a tyramide molecule is attached to any variety of molecules or particles that facilitate detection. For example, a tyramide molecule may be attached to a dye (e.g., a fluorescent dye). Therefore, the presence of the dye immobilized with respect to the capture object (e.g., via the immobilized labeling agent) can be used to detect the presence of an analyte molecule associated with such capture object. In some cases, the conversion of tyramide to activated tyramide may cause a component associated with the tyramide to become detectable (e.g., may cause a non-fluorescent component to fluoresce upon activation. Because HRP activates the tyramide molecules catalytically, the HRP component of a single binding ligand immobilized with respect to a capture object (e.g., via an analyte molecule or particle) can generate numerous activated tyramide molecules (some or all of which may form covalent bonds with or otherwise be come immobilized with respect to the capture object) if a sufficient amount of reactants are provided, which can form an amplified signal at the capture object. Additionally or alternatively, immobilized tyramides may form sites for immobilizing additional binding ligands comprising HRP components having affinity for the tyramides. The additionally bound HRP components can further activate tyramide molecules that become attached to the capture object, further amplifying the signal. For example, tyramide-biotin can be used to label the capture objects, followed by labeling with Streptavidin conjugated to dyes for fluorescence detection.

Another specific illustrative example of some embodiments involving conversion of a precursor labeling agent into a labeling agent immobilized with respect to a capture object via an enzymatic component of a binding ligand involves binding ligands having a component comprising a phosphatase. As a non-limiting example in which the analyte molecule is an antigen, a binding ligand may be a phosphatase-labeled antibody or streptavidin conjugate. Phosphatase components can be used, for example, to mediate Enzyme-Labeled Fluorescence (ELF) signal amplification. In ELF detection, a binding ligand may have either an alkaline phosphatase or an acid phosphatase component, and the precursor labeling agent comprises an ELF 97 phosphate molecule (2-(5′-chloro-2-phosphoryloxyphenyl)-6-chloro-4(3H)-quinazolinone). Exposure to the phosphatase component can convert the ELF 97 phosphate, which is a water-soluble molecule with a light blue fluorescence signal, to a water-insoluble ELF 97 alcohol having a bright yellow-green fluorescence. The water-insoluble ELF 97 can act as a labeling agent by forming a fluorescent precipitate that can become immobilized with respect to the capture object (e.g., upon deposition of the ELF 97 alcohol precipitate onto the capture object). Fluorescence from the ELF 97 precipitate on the capture object (or near to the capture object) can indicate that at least one analyte molecule or particle is associated with that capture object.

Another illustrative example of conversion of precursor labeling agents into immobilized labeling agents is the use of rolling circle amplification (RCA). In some such embodiments, a binding ligand (e.g., an antibody) comprising an oligonucleotide primer is capable of binding to an analyte molecule or particle (e.g., associated with a capture object such as a bead). Such a binding ligand may be, for example, an antibody with a single stranded DNA oligonucleotide primer attached to the antibody (e.g., on the end of a heavy chain of the antibody). The binding ligand comprising the oligonucleotide primer when immobilized with respect to the capture object can be exposed to a circular DNA template having a sequence complementary to the primer. The complementary sequence of the circular DNA template can be copied via conversion of incoming added nucleotides (precursor labeling agents) into copies of the complementary sequence (e.g., in the presence of DNA polymerase) that become attached to the binding ligand as an elongated oligonucleotide (or polynucleotide) strand. Numerous (e.g., hundreds) of such copies of the complementary sequence may be made using the circular DNA template resulting in relatively long polynucleotide strands immobilized with respect to the capture object (e.g., via the binding ligand). The resulting single-stranded polynucleotide strands may serve as labeling agents by having detectable moieties (in some instances numerous detectable moieties) such as fluorescent probes attached to added complementary nucleotides bound to some or all of the copied nucleotide sequence in the elongated polynucleotide strand.

In some embodiments, capture objects associated with immobilized labeling agents are spatially segregated (e.g. by being compartmentalized). In certain cases, the capture objects are compartmentalized into a plurality of assay sites that are in the form of reaction vessels (e.g., microwells). Such spatial segregation may occur prior to or after to the immobilization of the labeling agents.. The reaction vessels may be sealed in some embodiments, but can remain unsealed in other embodiments. In some embodiments, capture objects associated with immobilized labeling agents are confined in liquid droplets. In some such embodiments, the droplets are spatially segregated. In some such instances the droplets are arranged on a planar surface. In some such embodiments the droplets are temporally segregated with respect to a fixed detection location, for example by being flowed through a channel (e.g., during an addressing step) past such a detection location. In some embodiments, capture objects associated with immobilized labeling agents are spatially segregated across a planar surface (e.g., to form an ordered array or a random distribution of capture objects, depending on the specific format of the assay).

### Concentration of Analyte Molecules or Particles in a Fluid Sample and Sensitivity of the Assays

The methods and systems described may provide for techniques for detecting or quantifying analyte molecules or particles in fluid samples having relatively low concentrations of the analyte molecule or particles. In some embodiments, the concentration of the molecules or particles (e.g., a particular type of molecule or particle) in the fluid sample is less than or equal to 50×10⁻¹⁵ M, less than or equal to 10×10⁻¹⁵ M, less than or equal to 5×10⁻¹⁵ M, less than or equal to 1×10⁻¹⁵ M, less than or equal to 500×10⁻¹⁸ M, less than or equal to 100×10⁻¹⁸ M, less than or equal to 50×10⁻¹⁸ M, less than or equal to 10×10⁻¹⁸ M, less than or equal to 5×10⁻¹⁸ M, less than or equal to 2×10⁻¹⁸ M, and/or as low as 1×10⁻¹⁸ M, as low as 500×10⁻²¹ M, as low as 100×10⁻²¹ M, as low as 50×10⁻²¹ M, as low as 40×10⁻²¹ M, or less.

The methods or systems described herein may provide for assays for detecting or quantifying analyte molecules or particles in fluid samples characterized by relatively low levels of detection (LOD) for the analyte molecule or particle. The LOD of an assay generally refers to the concentration of the analyte molecule or particle at which the signal rises above three standard deviations over the background). In some embodiments, the assay methods are characterized by a level of detection for the analyte molecules or particles (e.g., a particular type of molecule or particle) of less than or equal to 50×10⁻¹⁵ M, less than or equal to 10×10⁻¹⁵ M, less than or equal to 5×10⁻¹⁵ M, less than or equal to 1×10⁻¹⁵ M, less than or equal to 500×10⁻¹⁸ M, less than or equal to 100×10⁻¹⁸ M, less than or equal to 50 ×10⁻¹⁸ M, less than or equal to 10×10⁻¹⁸ M, less than or equal to 5 ×10⁻¹⁸ M, less than or equal to 2 ×10⁻¹⁸ M, and/or as low as 1×10⁻¹⁸ M, as low as 500×10⁻²¹ M, as low as 100×10⁻²¹ M, as low as 50×10⁻²¹ M, as low as 40×10⁻²¹ M, or less.

As will be appreciated by those in the art, many types of analyte molecules and particles may be detected and, optionally, quantified using methods and systems described; basically, any analyte molecule able to be made to become immobilized with respect to a capture object can be potentially investigated using at least some of these methods and systems. Certain more specific targets of potential interest that may comprise an analyte molecule are mentioned below. The list below is exemplary and non-limiting.

In some embodiments, the analyte molecule is or comprises a protein. For example, the analyte molecule may be an enzyme. Non-limiting examples of enzymes include, an oxidoreductase, transferase, kinase, hydrolase, lyase, isomerase, ligase, etc. Additional examples of enzymes include, but are not limited to, polymerases, cathepsins, calpains, amino-transferases such as, for example, AST and ALT, proteases such as, for example, caspases, nucleotide cyclases, transferases, lipases, enzymes associated with heart attacks, etc. When a system/method herein is used to detect viral or bacterial agents, appropriate target enzymes include viral or bacterial polymerases and other such enzymes, including viral or bacterial proteases, or the like.

In other embodiments, the analyte molecule comprises an enzymatic component. For example, the analyte particle can be a cell having an enzyme or enzymatic component present on its extracellular surface. Alternatively, the analyte particle is a cell having no enzymatic component on its surface. Such a cell is typically identified using an indirect assaying method described below. Non-limiting example of enzymatic components are horseradish peroxidase, beta-galactosidase, and alkaline phosphatase.

In some embodiments, the analyte molecule comprises a biomolecule. Non-limiting examples of biomolecules include hormones, antibodies, cytokines, proteins, nucleic acids, lipids, carbohydrates, lipids cellular membrane antigens and receptors (neural, hormonal, nutrient, and cell surface receptors) or their ligands, or combinations thereof. Non-limiting embodiments of proteins include peptides, polypeptides, protein fragments, protein complexes, fusion proteins, recombinant proteins, phosphoproteins, glycoproteins, lipoproteins, or the like. As will be appreciated by those in the art, there are a large number of possible proteinaceous analyte molecules that may be detected or evaluated for binding partners using the present invention. Besides enzymes as discussed above, suitable protein analyte molecules include, but are not limited to, immunoglobulins, hormones, growth factors, cytokines (many of which serve as ligands for cellular receptors), cancer markers, etc. Non-limiting examples of biomolecules include PSA, TNF-alpha, troponin, and p24, IL-17A, IL-12p70, and interferon alpha (IFN-alpha).

In some embodiments, the analyte molecule is or comprises a biomarker. For example, the analyte may be or comprise a neurological biomarker. Examples of suitable neurobiological biomarkers include, but are not limited to, tau protein, neurofilament light (NF-L), glial fibrillary acidic protein (GFAP), and ubiquitin carboxyl-terminal hydrolase L1 (UCH-L1).

In certain embodiments, the analyte molecule is or comprises a post-translationally modified protein (e.g., phosphorylation, methylation, glycosylation) and the capture component comprises an antibody specific to a post-translational modification. Modified proteins may be captured with capture components comprising a multiplicity of specific antibodies and then the captured proteins may be further bound to a binding ligand comprising a secondary antibody with specificity to a post-translational modification. Alternatively, modified proteins may be captured with capture components comprising an antibody specific for a post-translational modification and then the captured proteins may be further bound to binding ligands comprising antibodies specific to each modified protein.

In some embodiment, the analyte molecule is or comprises a nucleic acid. A nucleic acid may be captured with a complementary nucleic acid fragment (e.g., an oligonucleotide) and then optionally subsequently labeled with a binding ligand comprising a different complementary oligonucleotide.

Suitable analyte molecules and particles include, but are not limited to small molecules (including organic compounds and inorganic compounds), environmental pollutants (including pesticides, insecticides, toxins, etc.), therapeutic molecules (including therapeutic and abused drugs, antibiotics, etc.), biomolecules (including hormones, cytokines, proteins, nucleic acids, lipids, carbohydrates, cellular membrane antigens and receptors (neural, hormonal, nutrient, and cell surface receptors) or their ligands, etc.), whole cells (including prokaryotic (such as pathogenic bacteria) and eukaryotic cells, including mammalian tumor cells), viruses (including retroviruses, herpesviruses, adenoviruses, lentiviruses, etc.), spores, etc.

The fluid sample containing or suspected of containing an analyte molecule may be derived from any suitable source. In some cases, the sample may comprise a liquid, fluent particulate solid, fluid suspension of solid particles, supercritical fluid, and/or gas. In some cases, the analyte molecule may be separated or purified from its source prior to determination; however, in certain embodiments, an untreated sample containing the analyte molecule may be tested directly. The source of the analyte molecule may be synthetic (e.g., produced in a laboratory), the environment (e.g., air, soil, etc.), a mammal, an animal, a plant, or any combination thereof. In a particular example, the source of an analyte molecule is a human bodily substance (e.g., blood, serum, plasma, urine, saliva, stool, tissue, organ, or the like). The volume of the fluid sample analyzed may be any suitable amount within a wide range of volumes, depending on factors such as, for example, the number of capture objects used/available, the number of locations us/available, etc. As mentioned above, in some embodiments, relatively large sample volumes are used compared to existing approaches.

### Integrated Microfluidic Consumables and Systems

As mentioned above, apparatuses for performing an assay may integrate some or all of the components described. For example, an apparatus may comprise a sample input component and a capture object reservoir (e.g., container, chamber). The apparatus may further comprise one or more reagent reservoirs, such as reservoirs (e.g., containers, chambers) for solutions comprising one or more binding ligands, some of which may comprise converting agents such as enzymatic components. In some embodiments, the apparatus comprises chambers for exposing capture objects to sample fluids (e.g., to allow incubation of capture objects with one or more analyte molecules or particles from the sample fluid). The apparatus may further comprise a sample washer configured to prepare capture objects and analyte molecules or particles from the fluid sample for detection (e.g., via one or more wash steps with a rinsing fluid). The sample washer may also be used for exposing the capture objects, some of which may be associated with at least one analyte molecule or particle, to one or more binding ligands and/or converting agents (e.g., enzymatic components). In some, but not necessarily all embodiments, the apparatus may comprise an assay consumable handler configured to be operatively coupled to an assay consumable. In some embodiments, the assay consumable handler and assay consumable are configured for immobilizing capture objects using methods described in this disclosure. For example, the assay consumable may have a surface comprising assay sites (e.g., each having a volume of between 10 attoliters and 100 picoliters). The assay consumable handler may further comprise a capture object applicator configured to apply the capture objects to the surface of the assay consumable or in proximity to the surface (e.g., in proximity to assay sites on the surface if present). In some such embodiments, the assay consumable handler further comprises a force field generator adjacent to the assay consumable and configured to generate a force field in proximity to the surface (e.g., in proximity of the assay sites). Further, the assay consumable handler may comprise a fluid injector configured to generate a fluid plug (e.g., comprising an aqueous solution) having a first meniscus and a second meniscus each adjacent to an immiscible fluid (e.g., a gas such as air) when on the surface of the assay consumable. However, in some embodiments, capture objects may be interrogated without being spatially segregated into different locations (e.g., assay sites), and the assay consumable may be configured for addressing, for example, capture objects comprising or contained within droplets surround by an immiscible fluid as described above. In some embodiments, the assay consumable handler comprises a fluid pump capable of moving fluid across the surface of the assay consumable. In some embodiments, the reservoirs for other reagents and/or components, such as precursor labeling agents (e.g., enzyme substrate) and sealing components (e.g., a liquid sealing component) are included in the assay consumable handler and/or an assay consumable. In some embodiments involving assay sites (e.g., as an array), the assay consumable handler may be configured to seal assay sites comprising immobilized capture objects and precursor labeling agents. The assay consumable handler may also comprise an imaging system comprising a detector and optics for detecting signals from the capture objects (e.g., from assay sites, from droplets, etc.). In some embodiments, the assay consumable handler further comprises a controller comprising one or more processors configured to modulate the fluid pump to move fluid across the surface of the assay consumable. The assay consumable handler may also comprise a computer-implemented control system configured to receive information from the imaging system and determine a measure indicative of a concentration of analytes or molecules. It should be understood that such integrated apparatuses may be in the form of, for example, automated robotic systems or in the form of microfluidic systems (e.g., with some or all of the components above present on a chip).

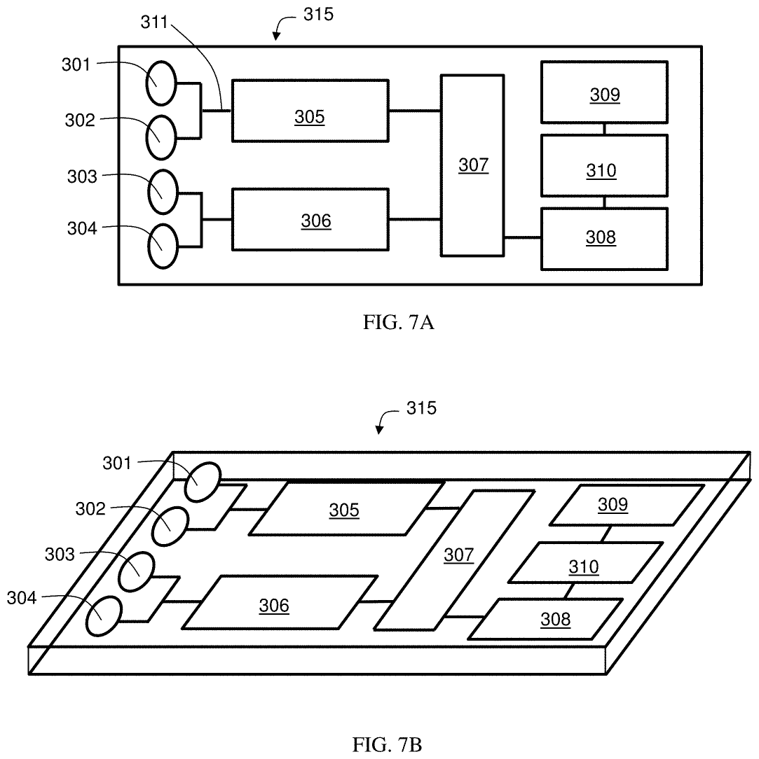

An integrated microfluidic apparatus configured to detect/quantify an analyte molecule or particle in a fluid sample may be in any of a variety of forms. In some embodiments, certain of the components described herein may be present on an assay consumable in the form of a microfluidic chip. FIGS. 7A-7B show a top view and perspective schematic illustration of one such embodiment, respectively. FIGS. 7A-7B show assay consumable 315 in the form of a microfluidic chip, according to certain embodiments. Assay consumable 315 comprises sample input chamber 301, capture object reservoir 302, binding ligand chamber 303, converting agent chamber 304, sample incubation chamber 305, binding ligand and converting agent incubation chamber 306, sample washer chamber 306, sealing component chamber 308, detection region 309, and precursor labeling agent chamber 310. Various of the chambers and regions of the microfluidic chip may be capable of forming fluidic connections via one or more microfluidic channels shown in FIGS. 7A-7B as solid lines such as solid line 311. Fluid movement may be accomplished using certain of the techniques described in this disclosure (e.g., negative and/or positive pressure differentials provided by a fluid pump (e.g., a vacuum), capillary flow techniques, electrophoretic techniques, digital microfluidics techniques (e.g., electrowetting on dielectric), etc.), and may be controlled by suitable configurations of valves and other microfluidic components known in the art. One embodiment of a suitable assay may comprise loading a sample fluid into assay consumable 315 via sample input chamber 301 and flowing a sample fluid from sample input chamber 301 to sample incubation chamber 305. Capture objects (e.g., beads) loaded into capture object chamber 302 (e.g., as pre-packaged capture objects or manually loaded capture objects) may also be made to flow into sample incubation chamber 305 (e.g., via a buffer solution). An incubation step may be performed in sample incubation chamber 305 where the capture objects may be exposed to analyte molecules or particles from the sample fluid and undergo an immobilization step as described in the assays above. Simultaneously or at a different point in time, a solution comprising a binding ligand in binding ligand chamber 304 and a solution comprising a converting agent (e.g., an enzymatic component) may each be made to flow into binding ligand and converting agent incubation chamber 306, where an incubation step (and subsequent association) may take place. Capture objects, at least some of which may be associated with at least one analyte molecule or particle, may be made to flow into sample washer chamber 307, where they may be combined with the incubated binding ligand/converting agents from chamber 306. In sample washer chamber 307, excess analyte molecules or particles and/or other solution components may be removed via one or more rinsing fluids (e.g., buffers) and may be allowed to associate with the binding ligands and converting agents. Once prepared, the capture objects may be directed to flow to detection region 309, where they may be interrogated. In some embodiments, the capture objects may be immobilized with respect to assay sites on a surface of assay consumable 315 in detection region 309, e.g., using immobilization methods described above involving force field generators and/or fluid plug flow with a receding meniscus. However, in some embodiments, such as certain embodiments in which the capture objects are isolated into separate liquid droplets surrounded by immiscible fluid in detection region 309, the capture objects may be interrogated as an array or as they flow through a channel (e.g., single file) past an imaging system operatively coupled to detection region 309 (not pictured). In some embodiments involving immobilization of capture objects with respect to assay sites in detection region 309, precursor labeling agent from precursor labeling agent chamber 310 may be introduced to detection chamber 309 following capture object immobilization. Further, in some embodiments, a sealing step may occur in which a sealing component (e.g., sealing liquid) from sealing component chamber 308 is flowed into detection region 309 following capture object immobilization, thereby sealing the assay sites (e.g., prior to detection). The imaging system and computer-implemented control system may then be used to acquire and analyze images and determine a measure indicative of a concentration of the analyte molecule or particle. In certain embodiments, a microfluidic chip as illustrated in FIGS. 7A and 7B may be designed to mate with, be manipulated by, and operated on by a robotic assay consumable handler. In other embodiments, such a microfluidic chip could be used individually and/or in a manual fashion by an operator.

In some embodiments, a microfluidic chip as illustrated in FIGS. 7A-7B is configured to associate, e.g. immobilize, capture objects with respect to assay sites (e.g., in detection region 309) using dielectrophoretic force from a non-uniform electric field, as described above. In some such embodiments, the fluid plug is transported to the detection region (e.g., detection region 309) using digital microfluidics techniques (e.g., electrowetting on dielectric techniques). For example, at least a portion of the detection region and/or channels of the microfluidic chip may comprise electrically conductive solids (e.g., electrodes) in conductive or inductive electrical communication with a power source and adjacent to the surface of the assay consumable. Application of voltage to the electrically conductive solids can cause movement of a fluid plug (e.g., from electrically conductive solid to electrically conductive solid) across at least part of a surface of the microfluidic chip (e.g., to the assay sites in detection region 309).

### Liquid Handling Techniques

The capture object-based assays described may be performed using preparation steps that can, in some instances, reduce or avoid loss of capture objects. As mentioned above, loss of capture objects during an assay may be particularly disadvantageous in assays employing relatively few capture objects. In some embodiments, one or more steps of an assay comprises mixing capture objects and analyte molecules or particles (e.g., associated or unassociated) in a liquid to form a capture object suspension, followed by removing the liquid. These steps may include initial exposure of the capture objects to a fluid sample, exposure of the capture objects to reagents (e.g., binding ligands), and/or wash steps. It has been determined in the context of this disclosure such liquid exposure and removal processes may be a source of loss of capture objects when performed using conventional liquid removal techniques. Certain liquid removal techniques (e.g., following sample washing) now described may avoid or reduce such loss of capture objects.

In some embodiments, capture objects may be provided. In some embodiments, relatively few capture objects are provided (e.g., less than or equal to 10,000, less than or equal to 5,000, and/or as few as 2,000, as few as 1,000, or fewer). These capture objects and analyte molecules or particles from a fluid sample may be prepared for detection. Preparing for detection may comprise one or more process steps comprising: (1) mixing the capture objects and analyte molecules or particles in a liquid to form a capture object suspension, and (2) applying a force to the capture object suspension to remove the liquid from the capture object suspension. In some embodiments, these preparation steps may be performed in suitable containers including, but not limited to, wells on plates (e.g., 96 well plates, 384 well plates, etc.), test tubes, Eppendorf tubes, etc.

In some embodiments, one such two-part process involves exposing the capture object to a fluid sample comprising the analyte molecules or particles, with that solution providing the liquid (e.g., an aqueous solvent such as a buffer or sample medium). The process would then involve removing the liquid from the resulting capture object suspension (e.g., to form a pellet of capture objects, at least some of which are associated with at least one analyte molecule or particle).

In some embodiments, one such two-part process involves a later step of re-suspending capture objects, at least some of which are associated with at least one analyte molecule or particle, in a solution comprising binding ligand, with that solution providing the liquid. The process would then involve removing the liquid from the resulting capture object suspension (e.g., to form a pellet of capture objects, at least some of which are associated with at least one analyte molecule or particle and at least one binding ligand).

In some embodiments, one such two-part process involves a washing step using a wash solution, with the wash solution providing the liquid. In certain embodiments, the wash solution is selected so it does not cause appreciable change to the configuration of the capture objects and/or analyte molecules or particles and/or does not disrupt any specific binding interaction between at least two components of the assay (e.g., a capture component and an analyte molecule or particle). In other cases, the wash solution may be a solution selected to chemically interact with one or more assay components. As will be understood by those of ordinary skill in the art, a wash step may be performed at any appropriate time point during the described methods. For example, the capture objects may be washed after exposing the capture objects to one or more solutions comprising analyte molecules, binding ligands, precursor labeling agents, or the like. As another example, following immobilization of the analyte molecules or particles with respect to a plurality of capture objects, the capture objects may be subjected to a washing step removing any analyte molecules not specifically immobilized with respect to a capture object. In some embodiments where the two-part process involves a washing step, the process would then involve removing the liquid from the wash solution (e.g., aqueous buffer) from the resulting capture object/wash solution suspension (e.g., to form a pellet of washed capture objects, at least some of which are associated with at least one analyte molecule or particle and/or at least one binding ligand).

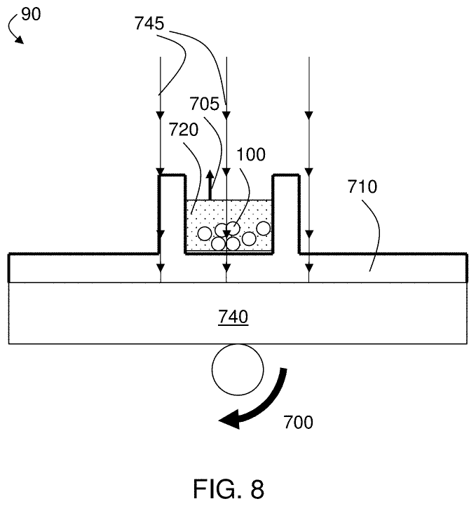

It has been determined in the context of this disclosure that application of certain forces to remove the liquid in the two-part processes described above may be performed in such a way that, in some instances, relatively few capture objects are lost. In particular, in some embodiments a force is applied to the capture object suspension, with that force not comprising applying a negative pressure to the capture object suspension via fluidic connection of the capture object suspension to a source of vacuum tending to remove the liquid. Fluidic connection of the capture object suspension to a source of vacuum tending to remove the liquid may include automated or manual pipetting/syringing supernatant liquid. However, such methods involving application of vacuum via a fluidic connection can, in some instances, pull capture objects from the suspension, providing a source of loss of capture objects. In contrast, applying other types of forces has been discovered to avoid such problems. For example, in some embodiments, a centrifugal force is applied to the capture object suspension, and the centrifugal force contributes to removal of the liquid. In some embodiments, apparatuses described comprise a sample washer configured to apply such a force to remove a wash solution from a capture object suspension. For example, referring to FIG. 8, sample washer 90 may be configured to apply a centrifugal force to capture object suspensions. Sample washer 90 may be configured to do so by comprising a force field generator capable of generating a force field in proximity to the capture objects acting on the capture objects such that the capture objects resist motion caused by the force applied to remove the liquid (e.g., a wash solution). As one example, FIG. 8 shows sample washer 90 comprising container 710 comprising capture objects 100 in liquid 720. Rotation of container 710 (as indicated by arrow 700) may cause centrifugal force 705 to remove liquid 720 from container 710. In some embodiments force field generator 740 (e.g., a magnet) can generate a force field (e.g., magnetic field) represented by vector field 745 acting on capture objects 100 (e.g., magnetic beads) so the capture objects resist motion caused by centrifugal force 705. Systems like sample washer 90 may be commercially available, such the Blue® Washer available from BlueCatBio, Inc.

In some embodiments, the sample washer comprises a force field generator capable of generating an electric field in proximity to the capture objects. The electric field may act on the capture objects. For example, the electric field may act on the capture objects such that the capture objects resist motion caused by the force field applied to remove the liquid. The electric field may also be used to facilitate other manipulations of the capture objects during sample preparation (e.g., in a microplate, in the sample washer, etc.), such as mixing, pelletization, and/or resuspension (e.g., following pelletization of the capture objects). In some embodiments, the force field generator is configurated to generate an electric field that acts on the capture objects using dielectrophoresis (e.g., by generating a non-uniform electric field). The force field generator may be configured such that the electric field can provide an attractive force or a repulsive force, depending, for example, on a frequency of the electric field (i.e., the frequency of a field from an alternating current). Such a configuration may allow for different dielectrophoretic forces to be applied to the capture objects at different points of the sample preparation process (e.g., to resist motion of the capture objects during liquid removal using positive dielectrophoresis and to promote motion of the capture objects when resuspension and/or mixing is desired using negative dielectrophoresis).

In some embodiments, the processes described above for preparing capture objects, at least some of which are associated with the analyte molecules or particles from the fluid sample and a statistically significant fraction of which are not associated with any analyte molecule or particle, may be performed so the total number of prepared capture objects is greater than or equal to 90%, greater than or equal to 95%, greater than or equal to 99%, or greater of initially provided capture objects. The prepared capture objects may then be used in a downstream step of an assay described. Some such steps may comprise determining a measure of the concentration of analyte molecules or particles in the fluid sample based at least in part on the measure indicative of the number or fraction of capture objects determined to be associated at least one analyte molecule or particle.

### Kinetic Principles

The inventors herein have determined in the context of this disclosure certain kinetic considerations that may provide for improved sensitivity of assays for detecting and/or quantifying analytes. In some instances, such kinetic considerations may contribute to assays having sensitivities in the ranges described above (e.g., less than or equal to 2 attomolar or even less). Some considerations relate to the recognition that sensitivity of an assay may scale with the efficiency with which analytes in solution are immobilized with respect to capture objects (i.e., extent of analyte capture). Such considerations may be of particular importance in some embodiments in which relatively few capture objects are employed, as analyte capture efficiency may be more difficult when few capture objects (and fewer capture components) are present. The inventors have recognized that in some embodiments, the affinity of a capture object (e.g., an affinity of a binding surface including capture components when present) can influence the extent to which analytes are captured under certain conditions. Therefore, in some, but not necessarily all embodiments, a capture object including a binding surface having a relatively high affinity for the analyte is used (e.g., a dissociation constant of less than or equal to 10⁻¹⁰ M, less than or equal to less than or equal to 10⁻¹¹ M, less than or equal to 10⁻¹² M, less than or equal to 10⁻¹³ M, or less). Further, as mentioned above, it has been determined in the context of this disclosure that relatively large sample volumes and relatively long durations of exposure of capture objects to fluid samples may be employed (e.g., using the ranges described above).

### Kits

Given the kinetic insight provided above and in the Examples below, it is possible to find conditions and select capture objects for such high sensitivity assays, including assays with relatively few capture objects. In some embodiments, kits are provided for preparing a sample of analyte molecules or particles for detection. The kit may comprise capture objects comprising a binding surface having affinity for the analyte molecule or particle. In some embodiments, the capture objects may be suitable for assays using relatively few capture objects (e.g., based on their affinity for the analyte, a density of capture components on their binding surfaces, or any of a variety of other considerations evident from this disclosure). In some embodiments a first assay using 5,000 capture objects identical to those in the kit has a limit of detection at least 50%, at least 75%, at least 90%, or least 99% lower than the limit of detection of a second assay using 500,000 capture objects identical to those in the kit under otherwise identical conditions except for the length of respective incubation steps for the first assay and the second assay. In some embodiments, the first assay comprises a step of incubating the capture objects with the analyte molecule or particle for a first period of time, while the second assay comprises a step of incubating the capture objects with the analyte molecule or particle for a second period of time, with the first period of time being substantially greater (e.g. 100 times greater) than the second period of time. “Otherwise identical conditions” includes conditions such as sample volume, sample source, detection conditions, etc., but does not include concentration of the capture objects in the sample. It should be understood that while the kit may be characterized by a comparison of limit of detection between assays with 500,000 versus 5,000 capture objects, the kit need not necessarily contain an amount of capture objects encompassed by these values. For example, the kit may have as few as 100 capture objects (or fewer) or as many as 5,000,000 capture objects (or more).

In some embodiments, a kit provided may comprise a packaged container for an analyte detection assay. Such a prepackaged container may comprise relatively few capture objects. The kit may be packaged for any of a variety of assays. In some embodiments, the kit is packaged for an assay involving up to 96 separate experiments (as would be performed by dividing the capture objects equally across wells of a 96-well plate). In some embodiments, the packaged container comprises greater than or equal to 50,000, greater than or equal to 100,000, greater than or equal to 500,000, greater than or equal to 1,000,000, and/or up to 2,000,000, or up to 5,000,000 capture objects, each including a binding surface having affinity for the analyte. The binding surface of the capture objects may, for instance, comprise a capture component having affinity for the analyte. The capture objects may be relatively small (e.g., having a diameter of between 0.1 micrometers and 100 micrometers). In some embodiments, the analyte detection assay can be performed at a relatively low limit of detection. For example, in some embodiments, the analyte detection assay can be performed at a limit of detection of less than or equal to 50×10⁻¹⁸ M, less than or equal to 50×10⁻¹⁸ M, less than or equal to 10×10⁻¹⁸ M, less than or equal to 5×10⁻¹⁸ M, less than or equal to 2×10⁻¹⁸ M, 5 less than or equal to 1 ×10⁻¹⁸ M, or less.

An exemplary apparatus for performing certain of the assays described herein is described. The apparatus may comprise a sample washer configured to prepare magnetic beads and analyte molecules or particles from a fluid sample for detection. In some, but not necessarily all instances, the sample washer is configured to remove liquid from a bead suspension without applying a negative pressure to the bead suspension (e.g., by instead applying a centrifugal force). The apparatus may further comprise an assay consumable handler configured to be operatively coupled to an assay consumable having a surface comprising reaction vessels (e.g., each having a volume of between 10 attoliters and 100 picoliters). The apparatus may further comprise a bead applicator configured to apply the magnetic beads to the surface of the assay consumable or in proximity to the surface. In some such embodiments, the apparatus further comprises a magnetic field generator adjacent to the assay consumable and configured to generate a magnetic field in proximity to the surface. Further, the apparatus may comprise a fluid injector configured to generate a fluid plug (e.g., comprising an aqueous solution) having a first meniscus and a second meniscus each adjacent to an immiscible fluid (e.g., a gas such as air) when on the surface of the assay consumable. In some embodiments, the apparatus comprises a fluid pump capable of moving fluid across the surface of the assay consumable. The apparatus may also comprise an imaging system comprising a detector and optics having a fixed field of view greater than an area defined by the array of reaction vessels. In some embodiments, the apparatus further comprises a controller comprising one or more processors configured to modulate the fluid pump to move fluid across the surface of the assay consumable (e.g., bi-directionally). The apparatus may also comprise a computer-implemented control system configured to receive information from the imaging system and analyze an entirety of the area containing the array of reaction vessels.

In some embodiments, a method for determining a measure of the concentration of analyte molecules or particles in a fluid sample is provided. The method may comprise exposing magnetic beads to a solution containing or suspected of containing at least one type of analyte molecule or particle. Some embodiments comprise immobilizing analyte molecules or particles with respect to the magnetic beads so at least some of the magnetic beads associate with at least one analyte molecule or particle from the fluid sample and a statistically significant fraction of the magnetic beads do not associate with any analyte molecule or particle from the fluid sample. In some instances, the solution is removed from at least a portion of the magnetic beads subjected to the immobilizing step. Some embodiments further comprise delivering the magnetic beads in proximity to reaction vessels on a surface (e.g., of an assay consumable). The method may further comprise generating a magnetic field in proximity to the surface acting on the capture objects so the capture objects move toward the surface (e.g., via a permanent magnet or an electromagnet). The method may also comprise flowing a fluid plug containing the magnetic beads so a receding meniscus of the fluid plug flows across at least some (or all) of the reaction vessels. The method may further comprise inserting at least a portion of the magnetic beads into the reaction vessels. Some embodiments comprise imaging an entirety of the reaction vessels following the inserting step and analyzing an entirety of the reaction vessels subjected to the imaging step to determine a measure indicative of the number or fraction of magnetic beads associated with an analyte molecule or particle from the fluid sample. In some instances, a measure of the concentration of analyte molecules or particles in the fluid sample is determined based at least in part on the measure indicative of the number or fraction of beads determined to be associated with at least one analyte molecule or particle.

In some embodiments, a method for determining a measure of the concentration of analyte molecules or particles in a fluid sample involving retaining a relatively high percentage of capture objects is provided. In some embodiments, the method comprises exposing capture objects to a solution containing or suspected of containing at least one type of analyte molecule or particle. The method may further comprise immobilizing analyte molecules or particles with respect to the capture objects so at least some of the capture objects associate with at least one analyte molecule or particle from the fluid sample and a statistically significant fraction of the capture objects do not associate with any analyte molecule or particle from the fluid sample. In some embodiments, the method further comprises removing the solution from at least a portion of the capture objects subjected to the immobilizing step while retaining at least 80%, at least 90%, at least 95%, at least 99%, or more of the capture objects subjected to the immobilizing step. At least 80%, at least 90%, at least 95%, at least 99%, or more of the capture objects subjected to the removing step may then be delivered in proximity to assay sites on a surface. In some embodiments, the method comprises immobilizing at least 20%, at least 30%, at least 40%, at least 50%, at least 60%, at least 75%, at least 90%, at least 95%, at least 99%, or more of the capture objects subjected to the delivering step with respect to the assay sites. The method may further comprise imaging at least 80%, at least 90%, at least 95%, at least 99%, or all of the assay sites. In some embodiments, the method comprises analyzing at least 75%, at least 90%, at least 95%, at least 99%, or all of the assay sites subjected to the imaging step to determine a measure indicative of the number or fraction of magnetic capture objects associated with an analyte molecule or particle from the fluid sample. The method may then comprise determining a measure of the concentration of analyte molecules or particles in the fluid sample based at least in part on the measure indicative of the number or fraction of capture objects determined to be associated at least one analyte molecule or particle.

U.S. Provisional Patent Application Ser. No. 63/010,613, filed Apr. 15, 2020, and entitled “Methods and Systems Related to Highly Sensitive Assays and Delivering Capture Objects,” and U.S. Provisional Patent Application Ser. No. 63/010,625, filed Apr. 15, 2020, and entitled “Methods and Systems Related to Highly Sensitive Assays and Delivering Capture Objects,” are each incorporated herein by reference in its entirety for all purposes.

The following examples are intended to illustrate certain embodiments of the present invention, but do not exemplify the full scope of the invention.

### EXAMPLE 1

This Example describes experimental procedures and modeling results related to increasing sensitivity of capture-object based assays according to certain embodiments.

The measurement of proteins is central to the life sciences, impacting basic research, diagnostics, and the development of therapeutics. Greater sensitivity of protein measurements (when combined with high specificity) can provide greater diversity in the proteins detected and the samples in which they are detected. In recent years, immunoassays based on the detection of single proteins have emerged as a promising approach to greatly improve the sensitivity of protein measurements, allowing for the detection of proteins at sub-femtomolar concentrations. Described in this Example is an approach to “digital” immunoassays based on the capture of proteins on microscopic superparamagnetic beads, labeling the proteins with enzyme labels, and detection of single enzyme labels by confining the beads and the product of the enzyme-substrate reaction within individual wells of an array of femtoliter-sized wells. This method was based on the classical enzyme-linked immunosorbent assay (ELISA) and digitized the read out of individual enzymes, so it has become known as digital ELISA. Digital ELISA has improved the sensitivity of immunoassays from picomolar (10⁻¹² M) down to subfemtomolar (˜10⁻¹⁶ M), and has been used broadly to allow new types of measurements of proteins. Most notably, digital ELISA has allowed for the detection of neurological biomarkers in plasma and serum, offering the potential of “blood tests for the brain” for the first time. Digital ELISA has also allowed for the measurement of inflammatory cytokines in the blood of healthy and diseased patients, and the detection of proteins important in the early and accurate diagnosis of infectious agents.

While digital ELISA has provided a technique for the measurement of proteins that were previously undetectable, it is clear that even greater sensitivity at low attomolar concentrations is needed. For example, the detectability of many cytokines (such as IL-17A) in blood is less than 100%, meaning that quantification of these molecules important in monitoring inflammatory status and response to anti-inflammatory therapeutics is not always possible in all healthy individuals. Furthermore, biological insights are possible by quantification of specific post-translational modification of proteins that provide greater biological and diagnostic specificity than the parent molecule, but often only represent a small fraction (˜1%) of the total concentration of the parent molecule. Detection of proteins in complex samples, such as stool and cerebrospinal fluid, can be accomplished by, for example, dilution of the sample in high concentrations of buffer to suppress so called matrix effects. Dilution, however, impacts detectability negatively, so more sensitive assays could allow for the detection of low abundance proteins in complex samples. Earlier detection of infectious diseases would also be possible by greater sensitivity to viral and bacterial proteins, e.g., HIV. Greater analytical sensitivity to proteins would also provide for detection in small sample volumes (e.g., blood from rodents, finger sticks, and heel sticks of pediatric patients), testing of less invasive samples that typically contain lower concentrations, and faster assays. The work described in this Example and the Examples below set out to increase the sensitivity of digital ELISA.

The approach to improving the sensitivity of digital ELISA was inspired by a model for the kinetics of the assay developed from a step-by-step analysis of assay efficiencies. In digital ELISA, superparamagnetic beads coated in capture antibodies are incubated with a sample containing the target protein. The proteins are bound with high efficiency for capture antibodies with high on-rates, and the proteins are statistically distributed over the beads according to Poisson distribution when [proteins]<[beads], as is the case at femtomolar concentrations and below. The beads are washed and incubated sequentially with a biotinylated detection antibody and streptavidin-β-galactosidase to label the immunocomplexes with single enzymes. The beads are resuspended in enzyme substrate and loaded into arrays of microwells, sealed with oil, and imaged to determine the fraction of beads associated with at least one enzyme. From this analysis, the average number of enzymes per bead (AEB) is determined via the Poisson distribution. A kinetic model of this process—based on the concentration of the different components, incubation times, and on- and off-rates of the different bimolecular interactions—predicted that AEB and, therefore, sensitivity, would increase as the number of beads decreased, with a desirable bead number of 10,000-50,000 beads for an antibody pair of good affinity. Previous tests of this model were limited to relatively large numbers of beads (500,000) because of the low efficiency of analyzing beads in the original digital ELISA, defined as “bead read efficiency”=(number of beads analyzed÷number of beads added to the sample). Typically only 5% of beads used to capture the protein from a sample were analyzed—about 25,000 beads—resulting in about 250 positive beads at the fraction of on beads (fₒₙ) at typical assay background. Because of low bead read efficiency, high input bead numbers were required to have sufficient positive beads at the limit of detection and avoid excessive Poisson noise. This Example and the Examples below demonstrate a method with greater bead read efficiency so lower numbers of capture beads (˜1,000-50,000) can achieve substantial increases in AEB and assay sensitivities. In this approach, the most sensitive assay would have a low number of beads for capture of proteins, and would be able to read as many of these beads as possible.

While certain existing approaches have increased the number of beads imaged, they have been limited in terms of improving the sensitivity of digital ELISA. First, past approaches used high bead numbers (hundreds of thousands to hundreds of millions) and did not examine the use of lower bead numbers (<10,000) determined to be advantageous in the context of the present disclosure to yield high sensitivity assays. Second, these approaches tailored their bead loading to increase the fraction of the wells filled, rather than a factor determined in the context of this disclosure to be relevant to assay sensitivity, namely, bead read efficiency. Finally, these prior approaches focused solely on the bead loading step of digital ELISA, and they did not examine the other steps in the process that impact the number of beads analyzed, such as the assay steps and image analysis.

In this and the following Examples, a method was developed that can improve the fraction of beads analyzed using low input bead numbers to improve the sensitivity of digital ELISA. An automated method for loading magnetic beads into microwell arrays with high efficiency based on a Simoa™ disk (Quanterix Corporation) and oil sealing is also described. A holistic approach was taken to improve bead read efficiency and examine each step in the assay, including loss of beads during the assay steps and image analysis. Based on improved bead read efficiencies, higher sensitivity digital ELISAs were developed for a number of different proteins, and the benefit in terms of detectability in clinical samples was demonstrated.

### EXPERIMENTAL

### Materials.

Capture antibody beads, detection antibodies, streptavidin-β-galactosidase (SβG), resorufin-β-D-galactopyranoside (RGP), wash buffers, sample diluent buffers, microtiter plates, pipette tips, and Simoa™ disks were obtained from Quanterix Corporation. Serum and plasma samples from healthy individuals were obtained from bioIVT.

### Assay Steps.

Digital ELISAs were performed following either three-step or two-step processes. In a three-step assay, samples were diluted in buffer, and diluted samples or calibrator solutions (100-250 μL) were added to each well of a 96 well microtiter plate. Solutions containing superparamagnetic beads coated in capture antibodies (25 μL) were then added to each well, and the plate was incubated on an orbital shaker (Quanterix Corporation) at 30° C. The beads in the wells were then washed using either the Simoa Washer™ (Quanterix Corporation) or the Blue® Washer (BlueCatBio) using a 96-well magnetic manifold to retain the beads during washing. The beads were then incubated sequentially with 100 μL of detection antibody and 100 μL of SβG, with washes between each step. At the end of the process the plates were left with the bead pellets dried on the 96-well magnetic manifold. Two-step assays were the same as three-step assays, except that detection antibody was added to the mixture of samples and beads for all or part of the sample incubation step, instead of a separate detection antibody step. Where needed to determine bead loss, bead numbers were quantified using a Multisizer Coulter counter particle analyzer (Beckman Coulter).

### Detection Using Simoa™ and Data Analysis.

The 96-well plate containing dried bead pellets was transferred to the SR-X™ reader (Quanterix Corporation) that performed Simoa readout of the assay beads. The SR-X™ was either used as received, or modified to perform a magnetic—meniscus sweeping bead loading protocol described below. On the SR-X™, the bead pellet was reconstituted in RGP using a disposable tip pipettor, and the RGP—bead mixture was transferred into an inlet port on the Simoa™ disk, where vacuum pulled the beads across the array of wells. The beads either settled or were actively loaded into the microwells, sealed with oil, imaged and analyzed to yield average enzymes per bead (AEB). AEB as a function of concentrations of calibrators were fitted to a four parameter logistic fit (4PL). Sample concentrations were determined by extrapolating their AEB values from these calibration curves. The limit of detection (LOD) of an assay was calculated as the concentration corresponding to signal three standard deviations above assay background, assuming a 10% coefficient of variation (CV) at assay background. The lower limit of quantification (LLOQ) and upper limit of quantification (ULOQ) were determined as the lower and upper limits of a calibration curve, respectively, where coefficient of variation (CV) profiling indicated imprecision in the concentration determined exceeded 20%. Dynamic range of the assays in the Examples herein was determined as log₁₀(ULOQ/LLOQ). CV profiling used an aggregate noise of the signal to calculate concentration imprecision. The aggregate noise was calculated by combining a fixed AEB CV of 7.1% and Poisson noise CV (from the number of beads analyzed) for each data point in the calibration curve. The imprecision in concentration was calculated as the CV of the concentrations interpolated from the 4PL fit of the mean signal, mean signal+noise, and mean signal−noise. This method of calculating LLOQ from calibration curves showed good correlation with LLOQs determined from imprecision in concentrations determined for serially diluted, low-concentration samples across at least ten runs (slope=0.83; r²=0.75).

### General Approach to Improving Sensitivity of Digital ELISA.

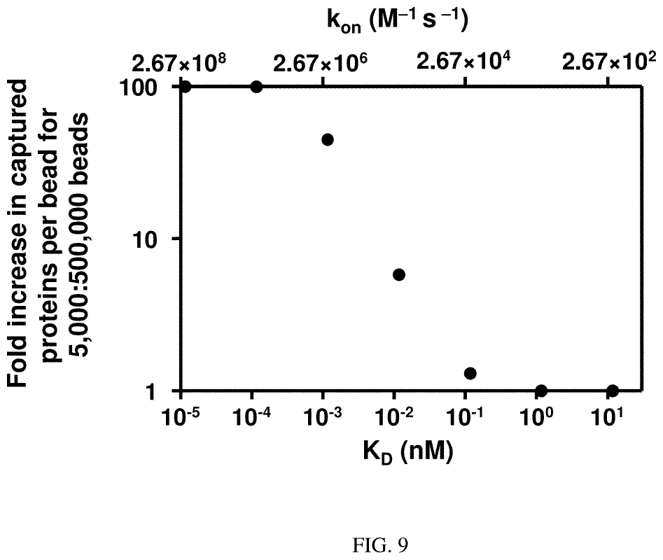

FIG. 9 shows a modeled increase in the ratio of target molecules to capture beads using 5,000 beads compared to 500,000 beads—assuming 274,000 antibodies per bead—as a function of the dissociation constant (K_{D}) of the capture antibody based on the kinetic model of Simoa. K_{D} was varied by changing the kₒₙ value at a fixed k_{off} (3.13×10⁻⁶ s⁻¹). As the subsequent labeling steps were not changed, these modeled increases were equal to the expected increase in AEB, i.e., assay slope. Assuming further that background AEB and imprecision did not change with bead number, FIG. 9 indicated improvements in LOD as a function of K_{D}. This model of sensitivity improvements assumed that diffusive—convective transport of target to the beads was not limiting, and that capture efficiencies could be modeled by considering only bimolecular reaction kinetics between the protein and capture antibodies. Given the long incubation time used for target capture used here, this assumption was believed to hold true and the modeling was valid for both low and high bead digital ELISAs. In this model, the main drivers of signals in digital ELISA were on-rate (kₒₙ) of the capture antibody—antigen interaction, and the amount of capture antibody on the beads.

The modeled changes in sensitivity as bead number was reduced tend to one of two limits (FIG. 9): a) improvement equal to the ratio of beads being compared (100× in this case) for high affinity antibodies (K_{D}≤10⁻¹³ M); and b) no improvement predicted (1×) for lower affinity antibodies (K_{D}≥10⁻⁹ M). The variation in sensitivity improvements was driven by the fraction of target proteins captured at equilibrium as a function of antibody concentration. In essence, binding to higher affinity antibodies can overcome the 100-fold drop in antibody concentration that resulted in using 100-fold fewer beads, and the protein-antibody binding reaction still went to completion over long incubation times. As these molecules were distributed over fewer beads, AEB increased by the ratio of the number of beads being compared, and improvements in sensitivity were realized. Lower affinity antibodies, however, became kinetically limiting at low antibody concentrations, and the protein-capture reaction reached equilibrium at a lower fraction of captured proteins. In this regime, the fraction of proteins captured was linear with antibody concentration, canceling the benefit of distributing proteins over fewer beads: AEB did not change, yielding no improvement in sensitivity.

This approach to improving the sensitivity of digital ELISA was based on two principles of assay design, the second of which stems from the insight in FIG. 9. First, the approach sought to capture every protein on the beads (protein capture efficiency=100%), and image every bead used to capture the proteins (bead read efficiency=100%). This principle should lead to every protein being detected and the most sensitive immunoassay possible. Second, the approach wanted to increase the signal-to-background (S/B) ratio of the assay (assay slope) by generating the highest AEB from capture beads bound to target molecules, i.e., increasing the ratio of molecules to beads. This principle favored reducing the number of beads used for capture and increasing volume of sample (increasing the number of molecules at same concentration). These two design principles, however, tend to work in opposite directions: rapid capture of proteins and low Poisson noise from reading beads favors high bead numbers and small volumes, whereas greater assay slope favors low bead numbers and large volumes. This approach was to balance these competing considerations to yield digital ELISAs with the highest sensitivities. Based on this two-pronged approach, the performance of each step of the digital ELISA process was improved accordingly. These improvements can be classified broadly as either improvements to bead read efficiency—summarized in Table 1 below—or improvements to the specific assay conditions that affect capture kinetics and assay slope (bead number, sample volume, and incubation time). Each of these are described in turn.

TABLE 1
Bead read efficiencies of standard digital ELISA and low bead
digital ELISA. Bead loss was measured for each step of both
assays as described elsewhere in the Examples resulting in
a predicted, aggregated number of output beads (B). The number
of beads remaining at the end of an assay using a known amount
of input beads (A) was also measured (C).

| | | Standard digital | | Low bead digital | |
|---|---|---|---|---|---|
| | | ELISA | | ELISA | |
| | Description | Bead | Number of | Bead | Number of |
| Step # | of step | loss | beads | loss | beads |
| | input beads | — | 500,000 | — | 5,540 |
| | (A) | | | | |
| 1 | assay steps | 12.1% | 439,500 | 0% | 5,540 |
| 2 | RGP-beads | 5.3% | 416,382 | 5.3% | 5,248 |
| | aspirated by | | | | |
| | tip | | | | |
| 3 | RGP-beads | 8.3% | 381,698 | 8.3% | 4,811 |
| | transferred | | | | |
| | to disk | | | | |
| 4 | beads loaded | 88.6% | 43,361 | 39.1% | 2,929 |
| | and sealed in | | | | |
| | wells | | | | |
| 5 | wells imaged | 30.8% | 30,027 | 1.6% | 2,881 |
| 6 | image | 10.0% | 27,028 | 6.7% | 2,688 |
| | analyzed | | | | |
| 7 | classification | 10.0% | 24,325 | 0% | 2,688 |
| | threshold | | | | |

| | | | | |
|---|---|---|---|---|
| output beads, cumulative (B) | — | 24,325 | — | 2,688 |
| efficiency, cumulative (B/A) | 4.9% | | 48.5% | |
| output beads, measured (C) | | 23,823 | | 2,614 |
| efficiency, measured (C/A) | 4.8% | | 47.2% | |

The approach to improving bead read efficiency was to reduce loss of beads at each step in the process. The assay process was broken into 6 steps—assay incubations and washings, aspiration of beads into a pipette tip, transfer of beads from tip to the Simoa™ disk, loading and sealing of beads in the wells, imaging of the beads in wells, and image analysis of the beads—and the loss of beads was determined at each of these steps. Analysis of each of these steps for previously published protocols using the SR-X™ indicated the bead efficiency for each step (Table 1), and a cumulative bead read efficiency of 4.9% that matched data generated from the overall assay (4.8%). These measurements closely matched the bead read efficiency of the original description of digital ELISA. Described in the next 5 sections is how each step was improved to increase bead read efficiency.

Based on increased bead read efficiency, it was possible to develop assays with low number of beads and higher sensitivity. To improve the sensitivity of these assays three important parameters were varied: number of capture beads, sample volume, and incubation time. While use of fewer beads increased the ratio of molecules to beads, doing so meant that the concentration of capture antibody was lower and beads were further apart, so longer incubation times were needed for high binding efficiencies to occur kinetically and to overcome diffusional transport of protein molecules to the beads. Similarly, greater sample volumes increased the ratio of molecules to beads, but required longer incubation times to ensure that protein molecules were captured. In subsequent sections, it is described how these assay conditions were determined.

### Reducing Bead Loss During the Assay Steps (Step 1).

Digital ELISA typically involves multiple cycles of pelleting beads on magnets, and removal of sample, detection antibody, enzyme conjugate, or wash buffer. Previously used methods for removing liquid from the wells were based on aspiration by needles pulling a vacuum. While this approach was effective at removing liquids, it was observed here that high shear forces generated from suction as the needles pass the pellet of magnetic beads can pull beads from the magnet and lead to beads being aspirated out of the reaction volume. Even a small loss of beads at each step can accumulate to a significant loss of beads over the 11 pellet-aspiration cycles in a three-step assay. Quantitative measurements showed that bead loss was 12.1% using the needle-based washing with 500,000 beads. Bead loss increased as the number of beads was reduced—as anticipated for superparamagnetic beads in a magnetic field—reaching 28.2% for 120,000 input beads. As this step was a significant contributor to loss in dilute solutions of beads, we developed assays based on a centrifugal removal of liquid from the wells of a microtiter plate rather than needle aspiration. This method showed excellent residual volumes via gravimetric methods (<1 μL) that is desirable for high sensitivity assays, and no bead loss over the entire assay process was detected within the error of the quantification method (<5%).

### Transfer of Beads to Simoa™ Disk (Steps 2 and 3).

The resuspension of beads in enzyme substrate and transfer of this solution to the Simoa™ disk can result in the loss of beads due to dead volumes. Gravimetric measurements showed that 38 μL of substrate was picked up by the tip and mixed with beads, and, as residual volume was required to be left behind in the well to avoid air in the tip, only 36 μL was transferred out of the well into the tip, resulting in a loss of beads of 5.3% (Step 2). Of these 36 μL, gravimetric measurements showed that 33 μL of RGP-bead mixture was transferred into the inlet port of the Simoa™ disk due to residual volume on the inside and outside of the tip, a loss of 8.3% (Step 3). Efforts to reduce the residual volumes at these two steps resulted in air being entrained in the disk and less reproducible bead loading, so no changes were made.

Loading of Beads into Microwells Using Magnetic Force Combined with Meniscus Sweeping (Step 4).

It was observed that the step in the original digital ELISA process that had the greatest loss of beads was the loading and sealing of beads into the array of microwells (Table 1). The original method relied on gravity loading and a settling time of 120 seconds that resulted in low efficiency of bead loading (˜11.5%). An improved method was developed to load as many beads as possible into microwells from dilute bead solutions.

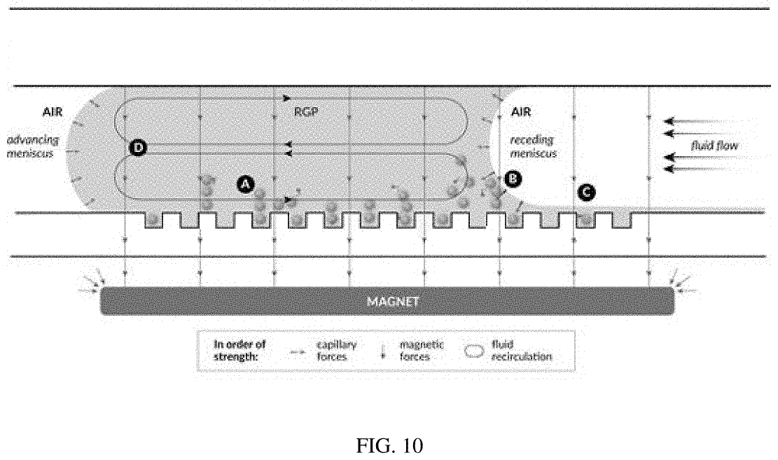

Magnets provide a promising way to move magnetic beads to the surface of the array of wells rapidly and to hold them in place once in the wells. The main challenge with using a magnet to load beads into microwells was that that superparamagnetic beads tended to chain in magnetic fields, and inter-bead attraction tended to prevent individual beads entering the microwells. Furthermore, a stationary magnet did not provide any lateral movement so beads did not move over the array surface and “sample” the opening of the wells. Fluidic flow of beads over the surface can increase bead loading by moving beads over wells, helping to wet the wells, and providing meniscus forces to drive beads into wells. Flow-driven loading of beads is limited in existing digital ELISA, however, because: a) flow occurs over the entire device rather than concentrating beads over the wells, so is intrinsically inefficient; b) it relies on gravity to initially move the beads to the surface of the array, so is inherently slow; and c) the oil sealing step pulls beads out of wells resulting in bead loss. To overcome the challenges of using magnets or flow alone, a method that combined magnetic forces and flow-induced capillary forces at the receding edge of the meniscus between air and liquid was developed (FIG. 10), to realize the benefits of both forces while counteracting their negative impact on bead loading.

FIG. 10 shows a schematic of one embodiment of the bead loading method based on a fixed magnet under the array of microwells that initially pulled the beads onto the surface, followed by multiple cycles of back-and-forth flow of the plug of RGP-bead solution over the array so the meniscus forces at the receding air-liquid interface push beads down into the microwells. FIG. 10 shows that (A) a vertical magnetic field causes beads to rapidly move to the surface, chain vertically via attractive forces, and repel in the plane. (B) capillary forces at the receding meniscus push beads down and into wells under flow, (C) capillary forces at thin films of liquid pin beads in the wells, and (D) inertial forces active during flow will cause aggregation at the receding meniscus and recirculation of beads. The solid arrows pointing from the menisci represent the strong capillary forces at the air-water interfaces. The vertical lines with arrows represent the magnetic field. The dotted lines show the weak inertial forces during flow. During experiments, once in the wells, the beads were held there magnetically during oil sealing. This method was called magnetic-meniscus sweeping (MMS). This approach led to improvements in bead loading efficiencies over gravity loading that could not be achieved using magnetic or meniscus forces alone. It is believed that loading beads into wells during MMS was driven by three forces operating on the beads:

### I. Magnetic Force Pulling Down and Chaining Beads.

The magnetic field perpendicular to the array rapidly pulls the beads to the surface of the array of wells, allowing beads to concentrate over the wells as the bead solution initially flows over the array. With a magnet placed close to the wells, the vertical magnetic field causes beads in the plane of the array to repel each other in the plane of the surface so the beads do not clump horizontally when starting from a uniformly dispersed bead suspension. In this configuration, bead chaining does occur, however, perpendicular to the array surface (A in FIG. 10). This orientation of chaining is favorable for subsequent de-chaining by capillary forces (II) without pulling beads out of wells.
Modeling of the magnetic field showed that for a magnet placed 0 mm from the wells, the beads would pellet on the edge of the array due to the strong local lateral fields at the edges of the magnet, and not be available to enter the wells. At 1 mm, the field lines were vertical and even across the array, in-plane repulsion was maximum so the beads have a low tendency to pellet, and it would be possible to pull the beads onto the array evenly across the surface within a few seconds. There was a trade-off between minimizing the lateral field component while maximizing vertical field component (which was proportional to lateral bead-bead repulsion): a distance of 1 mm provided a desirable balance of low lateral field component and large vertical field component. At ≥3 mm from the wells, the beads were pulled to the surface rapidly and chained vertically, but showed a greater propensity to pellet in the center of the array of wells because of lower in-plane repulsion. As the disk was 1.2 mm thick and clearance was required for the disk to move, based on the magnetic modeling a 0.4 mT NdFeB magnet with a greater area than the array that was positioned 1 mm below the disk was used, a total of 2.2 mm below the wells. Initial bead filling experiments showed this position gave the highest bead fill given mechanical constraints.
   II. Capillary Forces at the Receding Meniscus Pushing Beads into Wells.
The receding meniscus of an evaporating droplet can exert large forces on particles. These forces are at work as a solution of beads moves over an array of microwells: capillary forces at a receding meniscus can result in high efficiency loading of polystyrene beads into microwells on glass. In the assay of this Example, as the receding meniscus of RGP passed over a bead on the surface of the array, the contact angle was <90° and it exerted a force in the direction of flow and downwards towards the array surface (B in FIG. 10). At higher speeds, the surface normal at the trailing edge is closer to vertical, increasing the downwards force on the beads. If this force is applied to a single bead over a well it could rapidly drive the bead into the well and pin it there. If the meniscus encounters a vertical chain of beads (e.g., 2-3 beads high) it can force the lowest bead into well, whilst shearing the upper beads sideways; once the beads have been displaced through an angle of ˜60° they may cease to attract each other and instead repel each other. The trailing fluid film (C in FIG. 10) formed as the receding meniscus passes the array can also increase the chances of bead loading. If this film is thinner than 1 bead diameter the beads will experience a force that pushes them strongly downwards.

### III. Bulk Fluidic Forces Leading to Aggregation and Recirculation of Beads.

Capillary forces were the strongest acting on the beads during MMS, followed by the magnetic force, and weaker bulk fluidic forces may have also played a role in increasing the loading of beads into wells. Previous research described recirculating currents of beads moving in droplets caused by the balance of fluidic drag and gravitational forces on the beads (D in FIG. 10). Depending on the relative strength of these two forces, the beads either accumulated as a densely packed bead mass at the receding meniscus, or were recirculated over the wells as the fluid flowed over the array. For the former, these beads would be available at the receding meniscus to load into wells via capillary and magnetic forces (B in FIG. 10). For the latter, recirculated beads would be pulled to the surface and chain under the magnetic field that would drive further loading of beads into wells at the receding meniscus. Which regime would win would depend on the proximity of the magnet and the flow rate of the bead suspension.

Processes I-III of MMS increased the chances of each bead encountering the entrance of a well and being forced into and held within a well. The number of beads trapped in a well increased with each cycle of these processes, so multiple cycles were implemented, i.e., sweeping of the beads completely over the array of microwells and back. Sweeping also had the benefit of increasing the rate at which the substrate wetted the wells to facilitate beads entering the wells. Importantly for digital ELISA based on low bead numbers, the use of magnets combined with meniscus sweeping made it possible to load dilute solutions of beads: the magnet focused the beads in the area containing microwells and meniscus sweeping allowed them to sample wells frequently enough to be loaded, approaches not needed when using high bead numbers.

MMS was implemented to load beads by modifying an SR-X™ reader (Quanterix Corporation) typically used for conventional gravity loading of beads. Two 4 mm×5 mm×1 mm N50 nickel-plated magnets were stacked and placed under the platen holding the Simoa disk at positions used for loading the RGP-bead solution and oil into the disk. As described below, various parameters to improve the bead loading efficiency of MMS were explored, and the following protocol was settled on. During bead loading, 33 μL of RGP-bead solution was first transferred to the inlet port of an array assembly in the Simoa™ disk. The transferred bead solution was pulled into the channel and across the array by applying a negative pressure equivalent to 33 μL in volume at a speed of 40 μL/s. The bead solution settled for 15 seconds above the first stack of magnets. This time allowed for the beads to be pulled onto the surface of the array or into the femtoliter wells, and for air trapped inside the femtoliter wells to start to be displaced by the aqueous liquid (“wetting”) as described previously. The first phase of meniscus sweeping was started by pulling the bead solution entirely across the array of microwells by applying a vacuum equivalent to 66 μL in volume at a speed of 40 μL/s, so that the bead solution was pulled for an equivalent of 99 μL in total volume into the channel from the inlet port. The entire volume of bead solution was then pushed back into the inlet port via a positive pressure, creating a receding meniscus that generated the capillary forces to drive beads downwards into the wells as they flowed over the array of microwells. The meniscus sweeping cycles were repeated by pulling and pushing 99 μL of equivalent volume over the array 5 times, ending with the bead solution collected in the inlet port. This sequence (pulling the bead solution across the array, waiting for 15 s, completing the first pull-push cycle and repeating 5 sweeping cycles) was then repeated. Finally, the bead solution that collected in the inlet port of the array assembly after the preceding steps was pulled across the array and settled for 15 s. All of the meniscus sweeping was performed above the stationary magnets placed under the microwell array. The Simoa™ disk was then rotated 15° clockwise to place the array of microwells containing beads in a waiting position for 190 seconds to allow parallel processing of other arrays in the disk. The Simoa™ disk was then rotated 15° clockwise to the oil seal position positioned above the second stack of stationary magnets. The beads were sealed into the array of microwells by flowing a fluorocarbon oil to displace the aqueous bead solution from the array surface and entrap RGP and beads within the wells. Once sealed with oil, the Simoa™ disk was rotated 15° clockwise to the imaging position (Step 5). This protocol for MMS yielded a bead loading efficiency of ˜61% (Table 1). This efficiency compared to ˜15% for meniscus sweeping without a magnet and ˜5% with the magnet in place but no meniscus sweeping using 120,000 beads.

### Development of Bead Loading Using Magnetic Meniscus Sweeping (MMS).

Variables explored in improving the bead loading efficiency of MMS were: volume of RGP mixed with beads; flow speed of RGP-bead mixture over the array; and wait time of the beads over the array before sweeping began. Bead loading did not vary significantly when RGP volume was varied between 25 and 45 μL. This observation was attributed to the fact that the meniscus and magnetic forces drove and held beads in wells, so higher concentration of beads was not beneficial here as it was for loading based on gravity. Below 25 μL, however, the resuspension of beads in the microtiter plate before loading onto the disk was not effective; above 45 μL, there was an increased risk of pulling beads out of the outlet port of the Simoa™ disk. 33 μL was selected as the most robust volume to use. The speed of sweeping was a key driver of high bead loading with speeds ˜50 μL/s resulting in high bead fills. Greater speeds (100 μL/s) resulted in slightly higher, but less robust bead fill rates. Lower speeds (<20 μL/s) resulted in inhomogeneous loading of beads. 40 μL/s was selected as the most robust speed yielding consistent bead loading. Finally, the dwell time on the magnet was examined before sweeping began, and the number of sweeps (Table 2), and selected a dwell time of 15 s and 10 sweeps total.

TABLE 2
Bead loading efficiency as a function of dwell time on the
magnet before sweeping commenced, and number of sweeps.

| | Number of | Number of |
|---|---|---|
| | sweeps = 10 | sweeps = 5 |
| Time on magnet = 30 s | 55% | 48% |
| Time on magnet = 15 s | 78% | 53% |

### Imaging of Wells (Step 5).

The original Simoa™imager for digital ELISA was based on a custom microscope objective and CCD camera with a field-of-view (FOV) of 2.63 mm×3.51 mm that was smaller than the size of the array (3.15 mm×4.2 mm). This FOV, therefore, limited the number of wells imaged to about 167,000 of the 238,764 wells in the array, and consequently 31% of the beads in the wells were unavailable for analysis (Table 1). The imager used in this Example (SR-X™) was based on optics and a CMOS camera imager with a larger FOV (3.19 mm×4.36 mm), i.e., greater than the area of the microwell array, although the commercial image analysis method digitally crops that image to match the FOV of original imager (Table 1). In contrast, here the entire FOV afforded by the SR-X was used, and it was possible to image ˜234,800 of the wells, reducing the loss of beads at this step to 1.6%. The remaining loss of wells was due to slight radial misalignments on some arrays causing wells near the edge to fall outside of the FOV, and failure to discriminate wells because of light scattering of beads that remained on the surface of the array between wells.

### Image Analysis to Identify Beads (Steps 6 and 7).

Once images were acquired, they were analyzed to identify wells and beads within those wells (Step 6). Loss of beads at this step was due to the identification and exclusion of debris (e.g., bubbles or aggregated beads) to avoid erroneous signals. Typically, 220,000 of the 235,000 wells remained after removal of debris from analysis, i.e., a loss of about 6%. Slightly lower debris in the images from MMS loaded arrays were observed, compared to the original bead loading method (6.3% vs. 10%). The final step in identifying beads from the images was to apply a classification threshold that results in removal of the outermost beads in a population to avoid “false” beads being analyzed (Step 7). Previously, a threshold of 10% was used to ensure effective discrimination of multiplex beads. As the work here was focused on measurement of a single bead type, the threshold was relaxed to 0% and bead loss was avoided.

### Overall Bead Read Efficiency.

Based on measurement and improvements to each step of the assay, the cumulative bead read efficiency (determined by multiplying efficiencies of Steps 1-7) improved from 4.9% for conventional digital ELISA to 48.5%, an improvement of about 10-fold (Table 2). The cumulative improvements were reflected in direct measurements of bead numbers in the assay, where bead read efficiencies were 4.8% and 47.2% for conventional digital ELISA and the improved assay, respectively (Table 1). This improvement in bead read efficiency provided a way for assays to be developed using fewer capture beads than previously, and to determination of desirable assay parameters under those conditions as described in the Examples below.

### EXAMPLE 2

This Example describes development of a digital ELISA for IL-17A based on a low number of capture beads and high bead read efficiency, in accordance with certain embodiments.

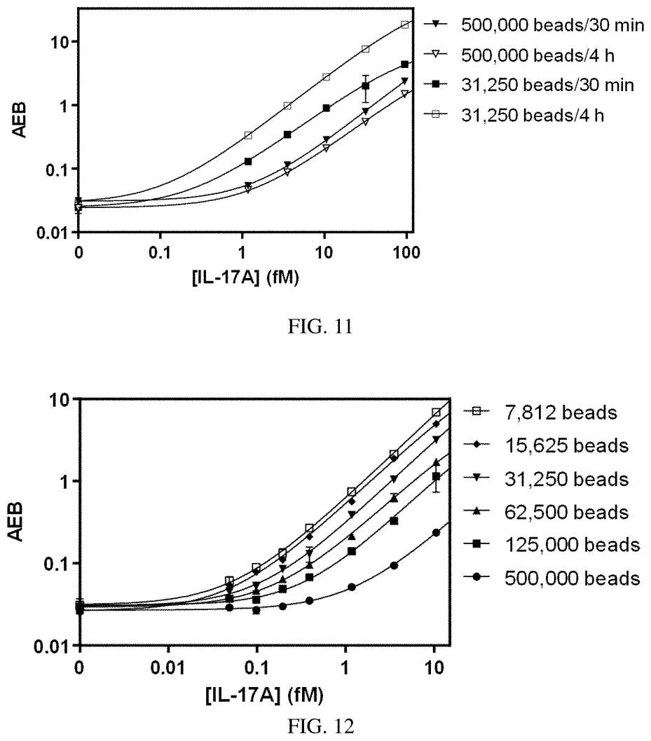

Based on the modeled improvements in sensitivity shown in FIG. 9, an assay with a capture antibody of high affinity was first tested. It was challenging to accurately determine the on- and off-rates for protein-antibody interactions at the surface of beads as the available analytical methods (e.g., SPR) used planar surfaces and different antibody immobilization chemistries to determine these values. As a surrogate for accurate kₒₙ values, IL-17A, which has one of the most sensitive digital ELISAs using 500,000 beads, was chosen, assuming that sensitivity was, in part, driven by a high affinity capture antibody. Furthermore, the reported detectability in serum and plasma of IL-17A using the digital ELISA was low (60%), so this assay would benefit from improved sensitivity. FIG. 11 shows a comparison of AEB for digital ELISAs for IL-17A using 500,000 and 31,250 beads per sample at two sample incubation times (30 min (standard) and 4 h). The data at 500,000 beads was generated using standard methods, and the data at 31,250 used the high bead efficiency digital ELISA process, including MMS. The solid lines are 4PL fits to the data. These data illustrate the increase in slope that results from using fewer beads to capture IL-17A: for conventional 30 min sample incubation, while the backgrounds for 500,000 and 31,250 beads were similar, the AEB using fewer beads increased so that the signal-to-background ratio at 1.2 fM increased 3-fold from 1.8 to 5.2. As a result, there was an improvement in LOD from 0.4 fM to 0.074 fM going from 500,000 to 31,250 beads under otherwise essential identical conditions. As anticipated by the kinetic model, the high bead number assay did not benefit from longer incubation times (S/B=1.8 and 2.0 at 1.2 fM for 30 min and 4 h incubation, respectively), as the target proteins were captured relatively rapidly, whereas the lower bead assay did benefit from longer incubation times (S/B=5.2 and 11.8 at 1.2 fM for 30 min and 4 h incubation, respectively), as the target proteins needed longer times to be captured on fewer beads.

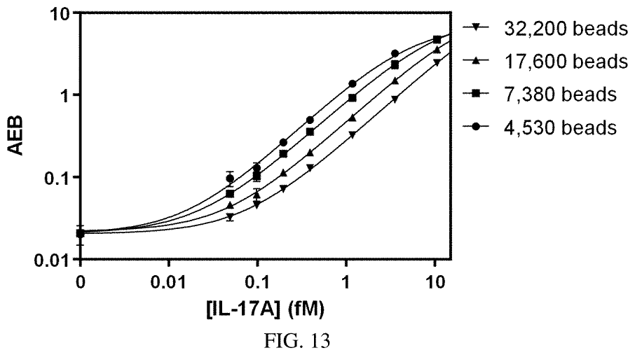

FIG. 12 show plots of AEB against [IL-17A] for digital ELISAs using 6 different bead numbers ranging from 7,810 to 500,000, and a 4 h sample incubation time. The data at 500,000 beads was generated using standard methods, and all the other conditions used the high bead efficiency digital ELISA process, including MMS. An outlier replicate was removed from the 500,000 bead data. Solid lines are 4PL fits to the data. These data show that the slopes continued to increase as bead number decreased down to <8,000 beads, while background did not change. As a result, LOD improved from 429 aM for 500,000 beads to 17 aM for 7,810 beads under the same conditions. The S/B ratio at the lowest concentration tested (49 aM) was 2.0 for 7,810 beads, whereas this concentration was not distinguished above background for the typical 500,000 bead assay. A further titration down to 1,200 beads (FIG. 13) showed that sensitivity continued to increase with fewer beads, but at 1,200 beads too few beads were loaded into wells to be imaged under the conditions of this experiment, and are not plotted in FIG. 13. Solid lines are 4PL fits to the data. As a result, ˜5,000 beads per sample was selected—100-fold lower than standard digital ELISA—as a robust number of beads giving higher sensitivity assays.

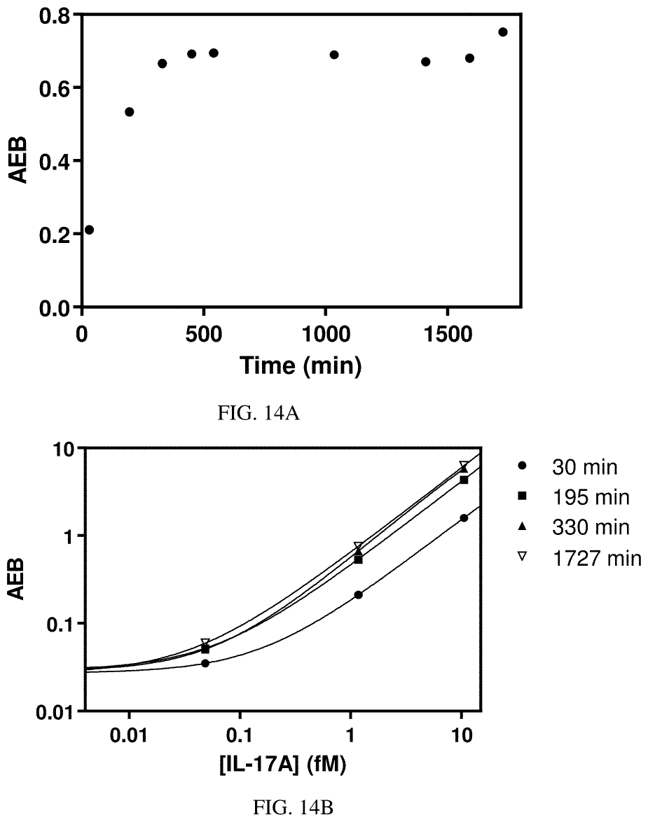

For high affinity antibodies, the kinetic model indicated that an increase in AEB from using fewer beads is equal to the fold decrease in the number of beads (FIG. 9). Table 3 shows the ratios of AEB at 10 fM (minus the background AEB) for bead numbers ranging from 7,812 beads to 125,000 beads as compared to the AEB at 10 fM (minus the background AEB) for 500,000 beads (data in FIG. 12). The fold increases in AEB were very close to the ratio of beads used down to 31,250 beads. This observation indicated that the IL-17A digital ELISA performed as modeled for a high affinity capture antibody (K_{D}≤10⁻¹³ M). At 15,625 beads and below, the increase in AEB was less than the fold decrease in the number of beads used, presumably because of incomplete capture at lower antibody concentrations. To ensure complete target capture at lower bead numbers, AEB was measured as a function of sample incubation time out to ˜29 hours using 15,000 beads. FIG. 14A shows AEB as a function of sample incubation time at 1.2 fM using 15,000 beads; FIG. 14B shows the calibration curves at 30, 195, 330, and 1727 min incubation times using 15,000 beads. Solid lines are 4PL fits to the data. These data indicate that capture was ˜25% complete after 30 min, ˜90% complete after about 6 h, and completed after >8 h. For assays performed during one workday, incubation times of 6 h were used; for highest sensitivity under these conditions, overnight incubations were used.

TABLE 3
AEB above background for different bead numbers,
and ratio of AEB increase and bead number compared
to the 500,000 bead condition. The data were taken
from FIG. 11 and FIG. 12. n.a. = not applicable.

| | | AEB ratio | Bead ratio |
|---|---|---|---|
| | AEB @ 10 fM- | to 500,000 | compared to |
| Condition | AEB @ 0 fM | beads | 500,000 beads |
| 500,000 | 0.1845 | n.a. | n.a. |
| 31,250 | 2.7415 | 14.9 | 16 |
| 500,000 | 0.2095 | n.a. | n.a. |
| 125,000 | 1.113 | 5.3 | 4 |
| 62,500 | 1.68305 | 8.0 | 8 |
| 31,250 | 3.1425 | 15.0 | 16 |
| 15,625 | 4.955 | 23.7 | 32 |
| 7,812 | 6.8505 | 32.7 | 64 |

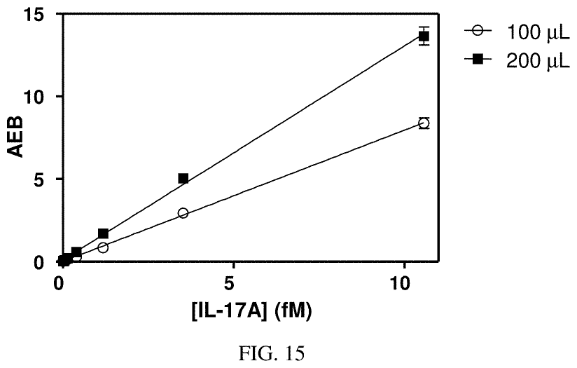

The volume of sample used in the assay was increased to improve sensitivity. In theory, increasing sample volume indefinitely for a fixed number of beads would lead to continuous improvements in sensitivity, although this approach is limited practically by the volume of the container the beads and sample are incubated in, and by the diffusion-convection-reaction kinetics of capture. In this case, sample incubations were performed in a 96-well plate with a maximum volume of 350 μL. The plates were shaken on an orbital shaker during incubations to keep beads suspended that limited the volume that could be used to 200-250 μL to avoid splashing between wells. FIG. 15 shows a comparison of assays of IL-17A using 100 and 200 μL of sample incubated with 15,000 beads for 16 h. Solid lines are linear fits to the data. The average increase in AEB above background was 88%, close to the doubling expected from theory. The LODs at 100 and 200 μL were 14 attomolar (aM) and 7 aM, respectively.

### Sensitivity of Improved Digital ELISA for IL-17A.

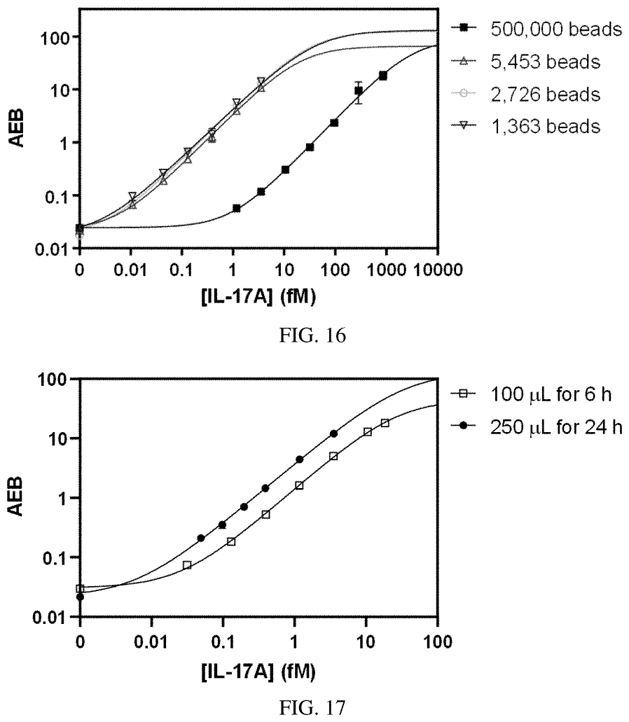

Based on adjustments of the assay described above, the highest sensitivity that could be achieved in a digital ELISA for IL-17A was determined. FIG. 16 shows calibration curves from digital ELISAs using 5,453, 2,726, and 1,363 capture beads, incubated with a 200 μL sample for 24 h, compared to a standard digital ELISA (500,000 beads incubated in a 100 μL sample for 30 min). Solid lines are 4PL fits to the data. Table 4 summarizes the LOD, LLOQ, ULOQ, and dynamic range of these 4 assay conditions. The lowest LOD (0.71 aM) and greatest improvement in LOD over standard digital ELISA (437-fold) was achieved using 1,363 beads. As bead number and the number of positive beads at background decreased, however, Poisson noise started to affect the data (Table 5), such that the best improvement in LLOQ over standard conditions (92-fold) was achieved using 5,453 beads. This phenomenon is illustrated by the increasing CVs from triplicate measurements and Poisson noise as bead numbers decreased (Table 5). The improvement in LOD of the assay using 5,453 vs. 500,000 beads (1.7 aM vs. 313 aM) corresponded well with that modeled by theory, whereas the lower bead number assays produced less of an improvement due to the effects of Poisson noise (Table 5). While assays with <5,000 beads may allow lower LODs, ˜5,000 capture beads yielded more robust and quantitative assays in the approach presented in this Example.

TABLE 4
LOD, LLOQ, ULOQ, and dynamic range of low bead digital
ELISAs for IL-17A plotted in FIG. 16 compared to
standard digital ELISA (500,000 beads).

| | 1,363 | | 2,726 | | 5,453 | | 500,000 | |
|---|---|---|---|---|---|---|---|---|
| | beads | | beads | | beads | | beads | |
| LOD | 0.72 | aM | 0.94 | aM | 1.7 | aM | 313 | aM |
| LLOQ | 11.9 | aM | 8.0 | aM | 7.6 | aM | 697 | aM |
| ULOQ | 53,773 | aM | 51,675 | aM | 33,353 | aM | 9,560,000 | aM |

| | | | | |
|---|---|---|---|---|
| Dynamic | 3.66 | 3.81 | 3.64 | 4.13 |
| range (logs) | | | | |
| Fold | 437 | 331 | 189 | — |
| improve- | | | | |
| ment in | | | | |
| LOD over | | | | |
| 500,000 | | | | |
| beads | | | | |
| Fold | 58.7 | 87.4 | 91.9 | — |
| improve- | | | | |
| ment in | | | | |
| LLOQ over | | | | |
| 500,000 | | | | |
| beads | | | | |

TABLE 5
Coefficient of variation (CV) of AEB and mean number of positive
beads over three triplicate measurements of background in low bead
digital ELISA data from FIG. 16. Four out of 21 arrays did not
produce an AEB for the 1,363 bead condition because of insufficient
beads, resulting in no CV being calculated for 3.52 fM.

| [IL-17A] (fM) | 5,453 beads | 2,726 beads | 1,363 beads |
|---|---|---|---|
| 3.52 | 3.3% | 2.5% | n.a. |
| 1.18 | 1.9% | 12.2% | 6.2% |
| 0.392 | 1.9% | 8.6% | 50.4% |
| 0.131 | 9.3% | 5.5% | 11.1% |
| 0.044 | 5.3% | 6.0% | 12.9% |
| 0.011 | 2.0% | 13.2% | 30.0% |
| 0 | 2.0% | 14.3% | 40.3% |
| Mean ± s.d. number of on | 49 ± 8 | 20 ± 1 | 8 ± 4 |
| beads (n) at [IL-17A] = 0 | | | |
| Poisson noise (\sqrt{n}/n) | 14% | 22% | 35% |
| at [IL-17A] = 0 | | | |

The greater slope (AEB per unit concentration) of digital ELISA using fewer beads means that the dynamic range of AEB (typically, 0.001<AEB<30) was spanned by a narrower range of concentrations, i.e., dynamic range of the concentrations measured was reduced by up to 0.5 log₁₀ (Table 4). In cases where greater dynamic range was required, it was possible to increase dynamic range via changes to image analysis algorithms and the modalities by which images were acquired.

FIG. 17 shows a comparison of assays of IL-17A designed to produce robust sensitivity (˜5,000 beads incubated in a 250 μL sample for 24 h) and an assay that might be more conveniently run in one day using less sample (˜5,000 beads incubated in a 100 μμL sample for 6 h). Solid lines are 4PL fits to the data. The LODs for these two assays were 1.8 aM and 7.4 aM, respectively, compared to 313 aM for the standard digital ELISA, i.e., improvements of 174-fold and 42-fold, respectively. In terms of beads imaged, the average (±s.d.) number of beads imaged was 2,700 (±397) over 21 arrays from 5,540 input beads, corresponding to a bead efficiency of 48.6%. In terms of the efficiency of detecting protein molecules, the overall molecular detection efficiency of the improved digital ELISA process was 13.2%±0.7%.

### Efficiency of Protein Detection Using Low Bead Digital ELISA.

From AEB values and the number of capture beads, the overall molecular detection efficiency of the digital ELISA process was determined. From the AEB values of the 6 concentrations of IL-17A measured by the 24 h/250 μL assay shown in FIG. 17, the average efficiency of capture and labeling of the protein on the beads (assay beads used×AEB/number of molecules) was 13.2%±0.7%. As just under 50% of the beads used were imaged, the average efficiency of detection of the target protein (beads imaged×AEB/number of molecules) was 6.4%±0.4%. From the experimental (FIG. 14A) and theoretical models of the kinetic of binding, close to 100% of the IL-17A were captured on the beads, so it can be inferred that only 1 in 7.6 of these molecules was labeled. This efficiency was limited by the non-specific binding of the labeling reagents (detection antibody and enzyme conjugate) to the capture beads. The efficiency could be increased by increasing the concentration of the labeling reagents, but assay background would also increase, yielding no benefits to S/B ratio and assay sensitivity.

### Adjustment of Spike Recovery and Dilution Linearity of IL-17A Digital ELISAs.

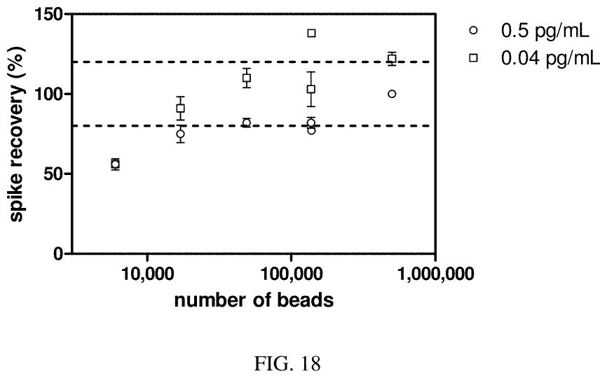

Robust immunoassays need to show consistent recovery of signals from spikes of known concentrations of target analyte into the sample type being tested (“spike recovery”), and linearity of the signals as the target analyte concentration is diluted (“dilution linearity”). FIG. 18 shows spike recovery of two concentrations of IL-17A in serum as a function of the number of beads used in the assay, using the dilution buffer and dilution factor of 4-fold used in an existing commercial IL-17A digital ELISA kit. Spike recoveries were acceptable (80-120%) for bead numbers ≥49,000 and decreased below these bead numbers, reaching 56% for 6,000 beads. To address the lower recoveries observed as bead number decreased, alternative dilution buffers and greater dilution factors were investigated. Increasing the serum and detergent content of the dilution buffer helped improve spike recovery and dilution linearity, suggesting that dilution buffers can be tailored for low bead assays. Increasing the sample dilution factor from 4-fold to 8-fold and adding bovine IgG to the standard dilution buffer improved spike recoveries at 0.12 pg/mL and 0.013 pg/mL to 87% and 88%, respectively. Dilution linearity was also acceptable using this buffer (Table 6). Sample testing proceeded using this buffer and dilution factor based on the improved spike recovery and dilution linearity, despite the extra 2-fold dilution reducing the effective sensitivity of the assay.

TABLE 6
Dilution linearity in serum and plasma of IL-17A starting with
an 8-fold sample dilution in standard buffer + bovine IgG.

| | Recovery | | |
|---|---|---|---|
| | Serum | Serum | Plasma |
| Dilution factor | Sample #1 | sample #9 | sample #18 |
| 16 | 79% | 105% | 85% |
| 32 | 98% | 114% | 132% |
| 64 | 108% | 109% | 161% |

### EXAMPLE 3

This Example describes detection of an analyte in different sample media using the methods described in Example 1, in accordance with certain embodiments. Higher sensitivity digital ELISA was used to measure IL-17A in the plasma of 50 individuals and the sera of 50 individuals. Before testing samples, the assay performance of the low bead digital ELISA was evaluated. Robust immunoassays need to show consistent recovery of signals from spikes of known concentrations of target analyte into the sample type being tested (“spike recovery”), and linearity of the signals as the target analyte concentration is diluted (“dilution linearity”). Once bead number decreased <49,000 beads, spike recovery in serum (but not sample diluent) decreased below acceptable limits (80-120%), with significant under recovery observed at 6,000 beads (FIG. 18). Additionally, dilution linearity in samples was outside of acceptable limits (80-120%). There were two methods to improve poor assay performance at low bead numbers: a) increase the matrix content of the calibrator diluent or b) increase the sample dilution factor. For sample testing with the low bead number assay, the sample dilution factor was increased from 4-fold to 8-fold, which improved spike recovery and dilution linearity to within acceptable ranges.

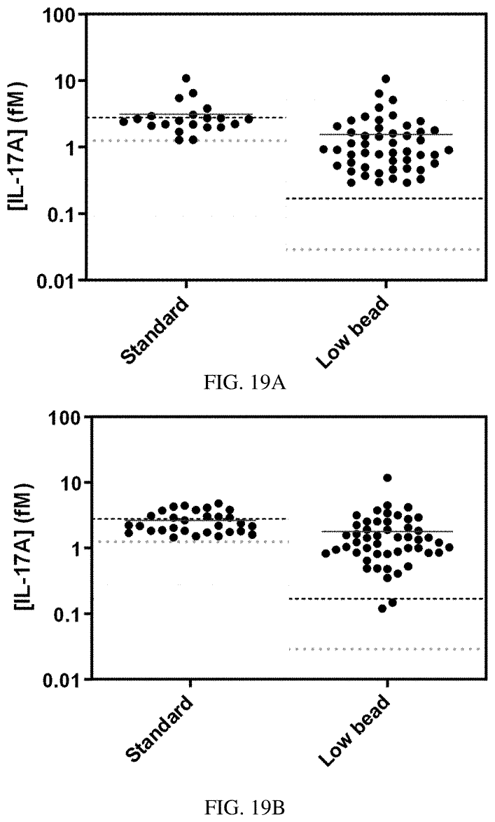

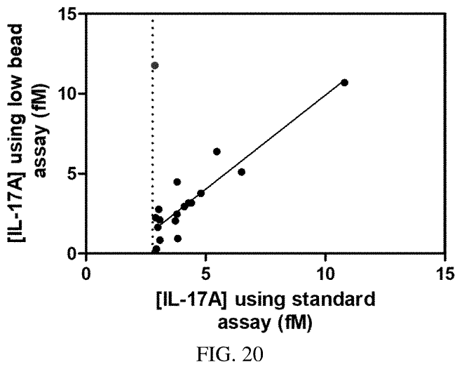

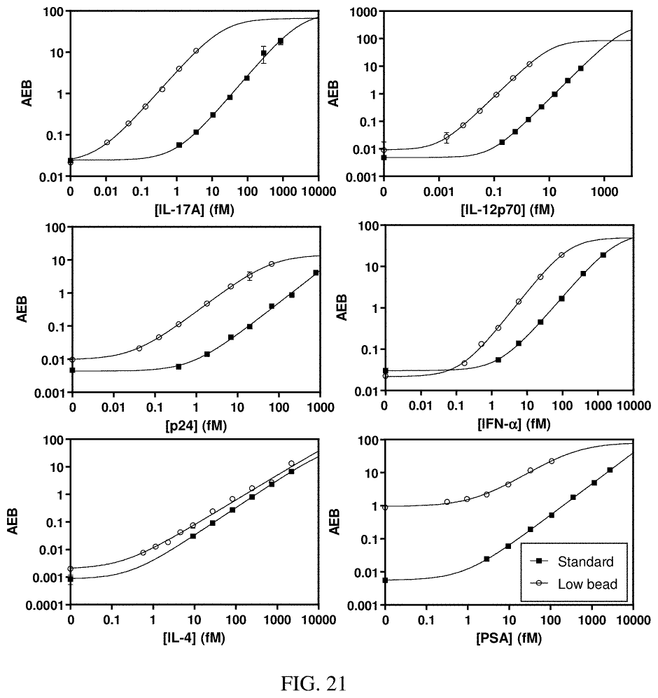

FIGS. 19A-19B shows scatter plots of the concentrations of IL-17A determined in these 100 serum and plasma samples using the standard digital ELISA (500,000 beads) and the more sensitive, low bead digital ELISA (5,000 beads). Specifically, FIGS. 19A-19B show 50 human plasma samples (FIG. 19A); and 50 human serum samples (FIG. 19B). The solid horizontal lines indicated the mean concentration of the samples. The dotted horizontal lines indicate detectable levels for the two assays (LOD×4 and LOD×8 for standard and low bead assay, respectively). The dashed horizontal lines indicate quantifiable levels for the two assays (LLOQ×4 and LLOQ×8 for standard and low bead assay, respectively). For the standard assay, IL-17A was quantifiable in 12% and 24% of plasma and serum samples, respectively, i.e., sample concentrations were above LLOQ×sample dilution factor (4). Furthermore, IL-17A was detectable in 42% and 60% (aggregate=51%) of plasma and serum samples, respectively, i.e., concentrations were above LOD×dilution factor (4). For the more sensitive, low bead assay, IL-17A was quantifiable in 100% and 96% of plasma and serum samples, respectively, i.e., concentrations were above LLOQ×dilution factor (8); IL-17A was detectable in 100% and 100% of plasma and serum samples, respectively, i.e., concentrations were above LOD×dilution factor (8). These data illustrated the ability of the low bead digital ELISA to significantly improve the detectability of cytokines in blood. FIG. 20 shows the correlation of concentrations determined by the standard and low bead digital ELISAs for the samples that were above LLOQ in both assays (n=18). The solid line is a linear regression fit to the data, excluding the outlier. The dotted line is the quantifiable limit in the standard assay (LLOQ×4). Excluding the one outlier, the correlation was good, showing a slope of 1.18 and an r² value of 0.85. The correlation was negatively impacted by many of the samples being close to the quantifiable limit on the standard digital ELISA.

### EXAMPLE 4

This Example describes detection of various protein analytes using the methods described in Example 1, according to certain embodiments. Having established the principle of improving the sensitivity of digital ELISA in Examples 2-3, assays were developed for 5 other proteins (IL-12p′70, p24, interferon alpha (IFN-alpha), IL-4, and prostate specific antigen (PSA)) using reagents from the existing commercial kits and conditions similar to IL-17A (FIG. 21). FIG. 21 shows plots of AEB against concentration of IL-17A, IL-12p′70, p24, IFN-α, IL-4, and PSA using digital ELISAs adjusted for low bead numbers (open circles) and standard digital ELISA (filled squares). Solid lines are 4PL fits to the data. The assay conditions for each protein are summarized in Table 7. The LODs, LLOQs, and improvements in sensitivity over standard digital ELISA are summarized in Table 8. Table 8 also shows the number of capture antibodies per bead. All of the proteins except IL-4 had greater than the number (274,000 per bead) used in the original model (FIG. 9); IL-4 had 17-fold fewer beads.

TABLE 7
Details of the assay conditions for the data shown in FIG. 21. All incubations were at 30° C.
IL-12p70 and p24 were 2 step assays, where detector was added to the sample-bead mixture.

| | Protein | | | | | | | | | | | |
|---|---|---|---|---|---|---|---|---|---|---|---|---|
| | IL-17A | | IL-12p70 | | p24 | | IFN-alpha | | IL-4 | | PSA | |
| | Format | | | | | | | | | | | |
| | 3-step | | 2-step | | 2-step | | 3-step | | 3-step | | 3-step | |
| Bead number | 500,000 | 5,453 | 400,000 | 5,368 | 300,000 | 5,250 | 250,000 | 5,966 | 300,000 | 5,500 | 500,000 | 5,043 |
| Sample volume | 100 μL | 200 μL | 100 μL | 200 μL | 125 μL | | 170 μL | | 100 μL | 200 μL | 100 μL | 200 μL |
| Sample | 30 min | 24 h | 45 min | 24 h | 45 min | 24 h | 30 min | 24 h | 30 min | 24 h | 10 min | 24 h |
| incubation time | | | | | | | | | | | | |

| | | | | | | |
|---|---|---|---|---|---|---|
| [Detection | 0.3 μg/mL | 0.3 μg/mL | 0.23 μg/mL | 2 μg/mL | 0.5 μg/mL | 0.33 μg/mL |
| antibody] | | | | | | |
| Detection | 10 min | 45 min | 45 min | 10 min | 10 min | 10 min |
| Antibody | | | | | | |
| incubation time | | | | | | |
| [SβG] | 150 pM | 150 pM | 300 pM | 100 pM | 50 pM | 50 pM |
| SβG incubation | 10 min | 10 min | 10 min | 10 min | 10 min | 10 min |
| time | | | | | | |

TABLE 8
Comparison of assay variables, LOD, and LLOQ for 6 standard commercially available digital ELISAs and corresponding
~5,000 bead digital ELISAs for data presented in FIG. 21. The highest theoretical fold improvement in LOD is for high
on-rate capture antibodies assuming ≥274,000 antibodies per bead = (ratio of sample volume used) × (ratio of beads used)⁻¹;
it does not account for Poisson noise or diffusion-limited binding. The minimum theoretical fold improvement in LOD =
(ratio of sample volume used). The PSA data had two outliers removed in low bead data.

| | | | | | | | | | | | Highest | Lowest |
|---|---|---|---|---|---|---|---|---|---|---|---|---|
| | | | | | | | | | | | theoretical | theoretical |
| | Number | | | | | Standard digital ELISA | | | | Fold | fold | fold |
| | of | Low bead digital ELISA | | | | (commercial kit) | | | | improve- | improve- | improve- |
| | antibodies | | Volume | LOD | LLOQ | | Volume | LOD | LLOQ | ment in | ment in | ment in |
| Protein | per bead | Beads | (μL) | (aM) | (aM) | Beads | (μL) | (aM) | (aM) | LOD | LOD | LOD |
| IL-17A | 1,172,000 | 5,453 | 200 | 1.7 | 7.6 | 500,000 | 100 | 313 | 697 | 189 | 183 | 2 |
| IL-12p70 | 466,000 | 5,368 | 200 | 0.31 | 2.4 | 400,000 | 100 | 22.3 | 114 | 72.6 | 149 | 2 |
| p24 | 333,000 | 5,250 | 125 | 9.1 | 52.8 | 300,000 | 125 | 242 | 1,292 | 26.6 | 57.1 | 1 |
| IFN-alpha | 592,000 | 5,966 | 170 | 45.9 | 112 | 250,000 | 170 | 528 | 1,256 | 11.5 | 41.9 | 1 |
| IL-4 | 16,000 | 5,500 | 200 | 37.6 | 1,053 | 300,000 | 100 | 80.5 | 1,208 | 2.1 | 109 | 2 |
| PSA | 652,000 | 5,043 | 200 | 423 | 1,373 | 500,000 | 100 | 238 | 900 | 0.6 | 198 | 2 |

For IL-17A, IL-12p70, and p24, the improvements in sensitivity were 189, 73, and 27-fold, respectively; these were consistent with capture antibodies with K_{D}<10⁻¹³ M (FIG. 9), with the improvement within 2-fold of predictions based on bead number and sample volume (Table 8). For IFN-alpha, the improvement was more moderate (11.5-fold), consistent with a capture antibody of lower affinity (K_{D} between 10⁻¹¹ and 10⁻¹² M). IL-4 improved only by the factor of the increase in sample volume, partly caused by the very low loading of capture antibodies on beads for this protein (Table 8): 16,000 antibodies per bead only results in a highest expected improvement of ˜30-fold. The digital ELISA for PSA behaved differently than the other 5 proteins, as background increased as the bead number was decreased, resulting in reduced sensitivity. This observation indicated there was a specific interaction between the detection antibody of PSA and the capture beads that increased AEB at background as bead number was reduced. This effect could be addressed by using an alternative detection antibody that does not have a specific interaction with the capture antibody. This limited screen of proteins indicated that one way to achieve consistent improvements by reducing the number of beads is by: a) engineering capture antibodies with K_{D}≤10⁻¹³ M; b) having a high loading of capture antibodies; and, c) reducing non-specific capture-detection interactions.

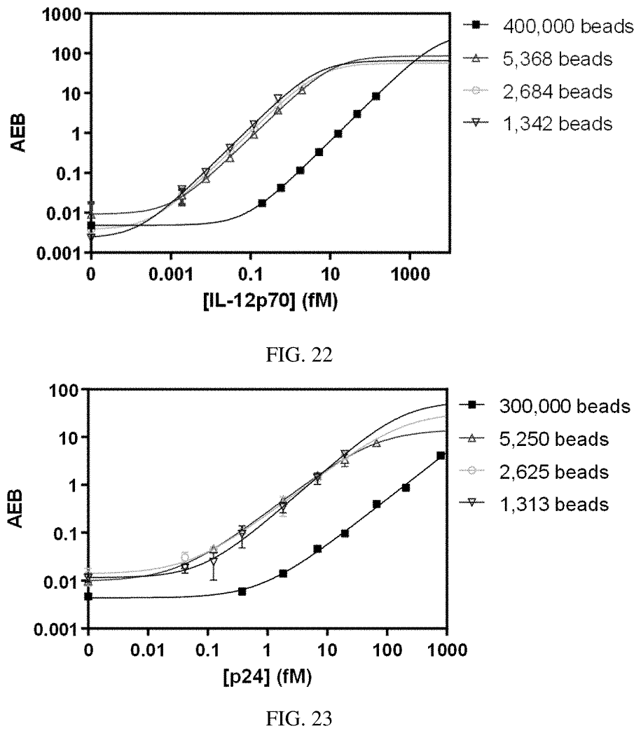

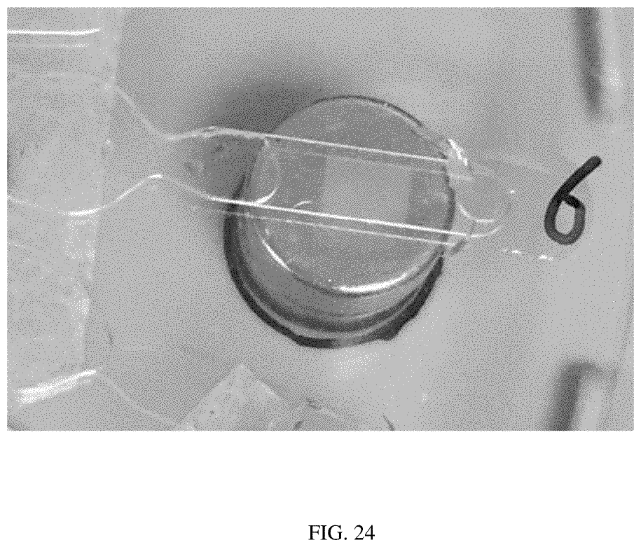

For IL-12p70 and p24, the lower limits on the number of beads used to further improve sensitivity were pushed. FIG. 22 and Table 9 show data from digital ELISAs for IL-12p70 down to 1,342 capture beads. Specifically, FIG. 22 shows plots of AEB against concentration of IL-12p70 spiked into diluted serum for standard ELISA (400,000 beads; 100 μL sample; 30 min incubation) and digital ELISAs adjusted for low bead numbers (5,368, 2,684, or 1,342 beads; 200 μL sample; 24 h incubation). Solid lines are 4PL fits to the data. As for IL-17A, the LOD increased at lower bead numbers, although better LLOQ were observed at higher bead numbers due to increasing Poisson noise from insufficient positive beads. The LOD using 1,342 beads was 45 zM or 5.5 molecules in 200 μL, an improvement of 486-fold over the standard digital ELISA, although only 48% of arrays had sufficient beads for analysis. The use of 2,684 beads was more robust with 100% of arrays having sufficient beads for analysis and an LOD of 92 zM. It is believed this assay is the most sensitive to a protein reported to date, and approached the limit of a single molecule in a sample. FIG. 23 and Table 10 show data from digital ELISAs for p24 down to 1,313 capture beads. Specifically, FIG. 23 shows

AEB against concentration of p24 spiked into diluted serum for standard ELISA (300,000 beads; 125 μL sample; 30 min incubation) and digital ELISAs adjusted for low bead numbers (5,259, 2,625, or 1,313 beads; 125 μL sample; 24 h incubation). Solid lines are 4PL fits to the data. In general, the p24 assay had lower precision than IL-17A and IL-12p′70, and further improvements to LOD were not realized at bead numbers below 5,000. The LOD using 5,250 beads was 9.1 aM, an improvement of 27-fold over the standard digital ELISA. This LOD was equivalent to -2.7 viruses per mL as each virus produces 2,000 copies of p24, compared to 20-25 viruses per mL for the most sensitive commercial PCR tests and 56 viruses per mL for early reports of digital ELISA. This improvement in sensitivity could allow earlier detection of infection by HIV than has been achieved previously using either nucleic acid testing or immunoassays.

TABLE 9
LOD, LLOQ, ULOQ, and dynamic range of low bead number
digital ELISAs for IL-12p70 plotted in FIG. 22 compared
to standard digital ELISA (500,000 beads).

| | 1,342 | | 2,684 | | 5,368 | | 500,000 | |
|---|---|---|---|---|---|---|---|---|
| | beads | | beads | | beads | | beads | |
| LOD | 0.046 | aM | 0.092 | aM | 0.31 | aM | 22.3 | aM |
| LLOQ | 3.2 | aM | 2.3 | aM | 2.4 | aM | 114 | aM |
| ULOQ | 6,676 | aM | 9,552 | aM | 21,601 | aM | 9,757,143 | aM |

| | | | | |
|---|---|---|---|---|
| Dynamic | 3.31 | 3.61 | 3.95 | 4.93 |
| range (logs) | | | | |
| Fold | 486 | 243 | 72.6 | — |
| improvement | | | | |
| in LOD over | | | | |
| 500,000 beads | | | | |
| Fold | 35.3 | 49.0 | 47.6 | — |
| improvement | | | | |
| in LLOQ over | | | | |
| 500,000 beads | | | | |

TABLE 10
LOD, LLOQ, ULOQ, and dynamic range of low bead number
digital ELISAs for p24 plotted in FIG. 23 compared
to standard digital ELISA (500,000 beads).

| | 1,313 | 2,625 | 5,250 | 500,000 |
|---|---|---|---|---|
| | beads | beads | beads | beads |
| LOD | 28.4 | 13.8 | 9.1 | 242 |
| LLOQ | 141 | 107 | 52.8 | 1,292 |
| ULOQ | 338,583 | 192,020 | 103,615 | 3,195,092 |
| Dynamic | 3.38 | 3.25 | 3.29 | 3.39 |
| range (logs) | | | | |
| Fold | 8.5 | 17.5 | 26.7 | — |
| improvement | | | | |
| in LOD over | | | | |
| 500,000 beads | | | | |
| Fold | 9.1 | 12.1 | 24.5 | — |
| improvement | | | | |
| in LLOQ over | | | | |
| 500,000 beads | | | | |

### EXAMPLE 5

This Example describes experiments and results relating to the flowing of fluid plugs containing magnetic beads across arrays of wells in combination with the application of a magnetic field.

Suspensions of 250,000 superparamagnetic beads were formed in 7.5 microliter aliquots of RGP. The bead suspension was applied to an array of microwells in a microchannel. The effect of the presence of a magnet and the use of meniscus flow was then studied. As shown in FIG. 24, a 10 mm by 9 mm neodymium iron boron (NeFeB) magnet was placed below the microwell array (239,000 wells in a 3.15×4.15 array, each well having a 44 fL volume) at varying distances. The aliquot was flowed so its meniscus passed over the array a variable number of times. The wells were then sealed with oil and imaged using a white light microscope. The images were then analyzed using Matlab™ to determine the percentage of wells filled with beads. Table 11 summarizes the results of the experiments.

TABLE 11
Well filling under various magnet and flow configurations.

| Magnet | | |
|---|---|---|
| (NeFeB φ10 mm) | Flow | Well filling |
| 1x magnet at 7 mm | Meniscus swept ~30x | 30%-40% |
| 2x magnet at 3mm | Meniscus swept ~30x | 90-95% |
| 2x magnet at 3 mm | Meniscus swept ~30x | 50% Bead |
| | | formed off array |
| | | on inlet side |
| 2x magnet at 1 mm | Simple transfer of | 0% Beads |
| | fluid to array | pelleted at edge |
| | | of array on loading |
| 2x magnet at 3 mm | Simple transfer of | 15% |
| | fluid to array | |
| 2x magnet at 3 mm | Simple transfer of | 15% |
| | fluid to array, | |
| | followed by oscillatory | |
| | flow without meniscus | |
| | touching array | |
| 2x magnet at 3 mm | Simple transfer of fluid | 80% |
| | to array, followed by | |
| | oscillatory flow without | |
| | meniscus touching array, | |
| | followed by meniscus | |
| | swept ~30x | |
| 2x magnet at 1 mm | No flow - 60s | 30-35% |
| after bead suspension | | |
| placed over array | | |
| 2x magnet at 1 mm | No flow - 60s | 70-80% fill |
| after bead suspension | followed by Meniscus | |
| placed over array | swept ~30x | |

The results summarized in Table 11 indicate that combination of magnetic force and meniscus sweeping results in more efficient insertion of beads than magnetic force in the absence of meniscus flow.

While several embodiments of the present invention have been described and illustrated herein, those of ordinary skill in the art will readily envision a variety of other means and/or structures for performing the functions and/or obtaining the results and/or one or more advantages described herein, and each of such variations and/or modifications is deemed within the scope of the present invention. More generally, those skilled in the art will readily appreciate that all parameters, dimensions, materials, and configurations described are exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, and in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of ” shall be closed or semi-closed transitional phrases, respectively, as set forth in the United States Patent Office Manual of Patent Examining Procedures, Section 2111.03.

1-90. (canceled)

91. A method for determining a measure of the concentration of analyte molecules or particles in a fluid sample, comprising:
exposing capture objects, each having affinity for a particular type of analyte molecule or particle, to a solution containing or suspected of containing at least one type of analyte molecule or particle, wherein the number of capture objects exposed to the solution containing or suspected of containing the analyte molecules or particles is less than or equal to 50,000;
immobilizing analyte molecules or particles of the particular type of analyte molecule or particle with respect to the capture objects such that at least some of the capture objects associate with at least one of the particular type of analyte molecule or particle from the fluid sample and a statistically significant fraction of the capture objects do not associate with any of the particular type of analyte molecule or particle from the fluid sample;
determining a measure indicative of the number or fraction of capture objects associated with at least one of the particular type of analyte molecule or particle from the fluid sample; and
determining a measure of the concentration of the particular type of analyte molecule or particle in the fluid sample based at least in part on the measure indicative of the number or fraction of capture objects determined to be associated with at least one of the particular type of analyte molecule or particle.

92. A method for determining a measure of the concentration of analyte molecules or particles in a fluid sample, comprising:
exposing capture objects, each having affinity for a particular type of analyte molecule or particle, to a solution containing or suspected of containing the at least one type of analyte molecules or particle, wherein the number of capture objects exposed to the solution containing or suspected of containing the analyte molecules or particles is less than or equal to 50,000;
immobilizing analyte molecules or particles of the particular type of analyte molecule or particle with respect to the capture objects such that at least some of the capture objects associate with at least one of the particular type of analyte molecule or particle from the fluid sample;
determining a measure indicative of the number or fraction of capture objects associated with at least one of the particular type of analyte molecule or particle from the fluid sample; and
based upon the measure indicative of the number or fraction of capture objects associated with at least one of the particular type of analyte molecule or particle from the fluid sample, either determining a measure of the concentration of the particular type of analyte molecule or particle in the fluid sample based at least in part on the measure indicative of the number or fraction of capture objects determined to be associated with at least one of the particular type of analyte molecule or particle, or determining a measure of the concentration of the particular type of analyte molecule or particle in the fluid sample based at least in part on a measured intensity level of a signal that is indicative of the presence of a plurality of the particular type of analyte molecules or particles.

93. A method for determining a measure of the concentration of analyte molecules or particles in a fluid sample, comprising:
exposing capture objects, each having affinity for a particular type of analyte molecule or particle, to a solution containing or suspected of containing at least one type of analyte molecule or particle;
immobilizing analyte molecules or particles of the particular type of analyte molecule or particle with respect to the capture objects such that at least some of the capture objects associate with at least one of the particular type of analyte molecule or particle from the fluid sample and a statistically significant fraction of the capture objects do not associate with any of the particular type of analyte molecule or particle from the fluid sample;
spatially segregating at least 25% of the capture objects subjected to the immobilizing step into a plurality of separate locations;
addressing at least a portion of the plurality of locations subjected to the spatially segregating step to determine a measure indicative of the number or fraction of capture objects associated with at least one of the particular type of analyte molecule or particle from the fluid sample; and
determining a measure of the concentration of the particular type of analyte molecule or particle in the fluid sample based at least in part on the measure indicative of the number or fraction of capture objects determined to be associated with at least one of the analyte molecule or particle.

94. A method for determining a measure of the concentration of analyte molecules or particles in a fluid sample, comprising:
exposing capture objects, each having affinity for a particular type of analyte molecule or particle, to a solution containing or suspected of containing at least one type of analyte molecule or particle, wherein the number of capture objects exposed to the solution containing or suspected of containing the analyte molecules or particles is less than or equal to 50,000;
immobilizing analyte molecules or particles of the particular type of analyte molecule or particle with respect to the capture objects such that at least some of the capture objects associate with at least one of the particular type of analyte molecule or particle from the fluid sample, while a statistically significant fraction of the capture objects do not associate with any of the particular type of analyte molecule or particle from the fluid sample;
immobilizing at least one binding ligand with respect to at least some of the particular type of analyte molecules or particles associated with a capture object;
exposing the at least one immobilized binding ligand to a precursor labeling agent such that the precursor labeling agent is converted to a labeling agent that becomes immobilized with respect to the capture object to which the binding ligand is immobilized;
determining a measure indicative of the number or fraction of capture objects comprising at least one immobilized labeling agent; and
determining a measure of the concentration of the particular type of analyte molecule or particle in the fluid sample based at least in part on the measure indicative of the number or fraction of capture objects determined to comprise at least one immobilized labeling agent.

95. The method of claim 93, wherein the number of capture objects exposed to the solution containing or suspected of containing the analyte molecules or particles is less than or equal to 50,000.

96. The method of claim 91, wherein the method further comprises:
spatially segregating at least a portion of the capture objects subjected to the immobilizing step into a plurality of separate locations;
addressing at least a portion of the plurality of locations subjected to the spatially segregating step to determine the measure indicative of the number or fraction of beads containing at least one of the particular type of analyte molecule or particle from the fluid sample.

97. The method of claim 91, wherein the method comprises spatially segregating at least 25% of the capture objects subjected to the immobilizing step into a plurality of separate locations.

98. (canceled)

99. The method of claim 91, wherein the method is characterized by a level of detection for the particular type of analyte molecule or particle of less than or equal to 50×10⁻¹⁸ M.

100. The method of claim 91, wherein the number of capture objects exposed to the solution containing or suspected of containing the analyte molecules or particles is less than or equal to 10,000.

101. The method of claim 91, wherein the number of capture objects exposed to the solution containing or suspected of containing the analyte molecules or particles is less than or equal to 7,500.

102. The method of claim 91, wherein the number of capture objects exposed to the solution containing or suspected of containing the analyte molecules or particles greater than or equal to 100.

103. The method of claim 91, wherein a ratio of the number of capture objects exposed to the solution containing or suspected of containing the analyte molecules or particles to the number of separate locations is less than or equal to 1:1.

104. (canceled)

105. The method of claim 91, wherein the solution containing or suspected of containing at least one type of analyte molecule or particles has a volume of greater than or equal to 50 microliters.

106. (canceled)

107. The method of claim 91, wherein the capture objects include a binding surface having affinity for the particular type of analyte molecule or particle.

108. The method of claim 91, wherein the capture objects comprise beads.

109.-126. (canceled)

127. The method of claim 91, wherein the capture objects are first capture objects and the particular type of analyte molecule or particle is a first type of analyte molecule or particle, and the method further comprises:
exposing second capture objects, each including a binding surface having affinity for a second type of analyte molecule or particle, to the solution containing or suspected of containing at least one type of analyte molecule or particle, wherein the number of second capture objects exposed to the solution containing or suspected of containing the analyte molecules or particles is less than or equal to 50,000;
immobilizing analyte molecules or particles of the second type of analyte molecule or particle with respect to the second capture objects such that at least some of the second capture objects associate with at least one of the second type of analyte molecule or particle from the fluid sample and a statistically significant fraction of the second capture objects do not associate with any of the second type of analyte molecule or particle from the fluid sample;
determining a measure indicative of the number or fraction of capture objects associated with at least one of the second type of analyte molecule or particle from the fluid sample; and
determining a measure of the concentration of the second type of analyte molecule or particle in the fluid sample based at least in part on the measure indicative of the number or fraction of capture objects determined to be associated at least one of the second type of analyte molecule or particle.

128-171. (canceled)

172. A method for determining a measure of the concentration of analyte molecules or particles in a fluid sample, comprising:
exposing capture objects to a solution containing or suspected of containing at least one type of analyte molecule or particle;
immobilizing analyte molecules or particles with respect to the capture objects such that at least some of the capture objects associate with at least one analyte molecule or particle from the fluid sample and a statistically significant fraction of the capture objects do not associate with any analyte molecule or particle from the fluid sample;
removing the solution from at least a portion of the capture objects subjected to the immobilizing step while retaining at least 80% of the capture objects subjected to the immobilizing step;
delivering at least 80% of the capture objects subjected to the removing step in proximity to assay sites on a surface;
immobilizing at least 20% of the capture objects subjected to the delivering step with respect to the assay sites;
imaging at least 80% of the assay sites;
analyzing at least 75% of the assay sites subjected to the imaging step to determine a measure indicative of the number or fraction of magnetic capture objects associated with an analyte molecule or particle from the fluid sample; and
determining a measure of the concentration of analyte molecules or particles in the fluid sample based at least in part on the measure indicative of the number or fraction of capture objects determined to be associated at least one analyte molecule or particle.

173-196. (canceled)

197. A kit for preparing a sample of analyte molecules or particles for detection, comprising:
capture objects including a binding surface having affinity for the analyte molecule or particle, wherein a level of detection of a first assay using 5,000 capture objects identical to those in the kit has a level of detection that is at least 50% lower than the level of detection of a second assay using 500,000 capture objects identical to those in the kit, wherein:
the first assay comprises a step of incubating the capture objects with the analyte molecule or particle for a first period of time,
the second assay comprises a step of incubating the capture object with the analyte molecule or particle for a second period of time, the first period of time being 100 times greater than the second period of time, and
the first assay and the second assay are performed under otherwise identical conditions.

198. A kit comprising a packaged container for an analyte detection assay, comprising between 50,000 and 5,000,000 capture objects each including a binding surface having affinity for the analyte and having an average diameter of between 0.1 micrometers and 100 micrometers, wherein the analyte detection assay can be performed at a level of detection of less than or equal to 50×10⁻¹⁸ M.

199. (canceled)

200. A composition, comprising:
an isolated fluid having a volume of between 10 and 1000 microliters;
at least one type of analyte molecule or particle present in a concentration of between 0.001 aM and 10 pM;
between 100 and 50,000 capture objects including a binding surface having affinity for the at least one type of analyte molecule or particle.
